(19) United States
(12) Patent Application Publication
(10) Pub. No.: US 2014/0277249 A1
(43) Pub. Date: Sep. 18, 2014

(54) Selectively Reducing Excess Consumption and/or Absorption of Unhealthy Food using Electrical Stimulation

(71) Applicant: Robert A. Connor, Forest Lake, MN (US)

(72) Inventor: Robert A. Connor, Forest Lake, MN (US)

(21) Appl. No.: 13/797,972

(22) Filed: Mar. 12, 2013

(51) Int. Cl. A61F 5/00

(52) U.S. Cl. CPC A61F 5/0026
USPC 607/40

(57) ABSTRACT

This invention can be embodied in a device and method for selectively reducing a person's excess consumption of one or more selected (unhealthy) nutrients, or foods containing such nutrients, using electrical stimulation. When embodied as a device, this invention includes a specific-nutrient-identifying sensor, a gastrointestinal electrical stimulator, and a cumulative-nutrient-consumption regulator. This invention provides reduced excess consumption of unhealthy nutrients and foods, while still allowing normal consumption of healthy nutrients and foods.

### CROSS-REFERENCE TO RELATED APPLICATIONS

This patent application claims the priority benefit of U.S. Provisional Patent Application No. 61/752,544 entitled “Selectively Reducing Excess Consumption and/or Absorption of Unhealthy Food using Electrical Stimulation” filed on Jan. 15, 2013 by Robert A. Connor of Medibotics, LLC.

### FEDERALLY SPONSORED RESEARCH

Not Applicable

### SEQUENCE LISTING OR PROGRAM

Not Applicable

### BACKGROUND

### Field of Invention

This invention relates to energy balance, weight loss, and proper nutrition.

### INTRODUCTION TO ENERGY BALANCE AND PROPER NUTRITION

The United States population has some of the highest prevalence rates of obese and overweight people in the world. Further, these rates have increased dramatically during recent decades. In the late 1990's, around one in five Americans was obese. Today, that figure has increased to around one in three. It is estimated that around one in five American children is now obese. The prevalence of Americans who are generally overweight is estimated to be as high as two out of three.

This increase in the prevalence of Americans who are overweight or obese has become one of the most common causes of health problems in the United States. Potential adverse health effects from obesity include: cancer (especially endometrial, breast, prostate, and colon cancers); cardiovascular disease (including heart attack and arterial sclerosis); diabetes (type 2); digestive diseases; gallbladder disease; hypertension; kidney failure; obstructive sleep apnea; orthopedic complications; osteoarthritis; respiratory problems; stroke; metabolic syndrome (including hypertension, abnormal lipid levels, and high blood sugar); impairment of quality of life in general including stigma and discrimination; and even death.

There are estimated to be over a quarter-million obesity-related deaths each year in the United States. The tangible costs to American society of obesity have been estimated at over $100 billion dollars per year. This does not include the intangible costs of human pain and suffering. Despite the considerable effort that has been focused on developing new approaches for preventing and treating obesity, the problem is growing. There remains a serious unmet need for new ways to help people to moderate their consumption of unhealthy food, better manage their energy balance, and lose weight in a healthy and sustainable manner.

Obesity is a complex disorder with multiple interacting causal factors including genetic factors, environmental factors, and behavioral factors. A person's behavioral factors include the person's caloric intake (the types and quantities of food which the person consumes) and caloric expenditure (the calories that the person burns in regular activities and exercise). Energy balance is the net difference between caloric intake and caloric expenditure. Other factors being equal, energy balance surplus (caloric intake greater than caloric expenditure) causes weight gain and energy balance deficit (caloric intake less than caloric expenditure) causes weight loss.

Since many factors contribute to obesity, good approaches to weight management are comprehensive in nature. Proper nutrition and management of caloric intake are key parts of a comprehensive approach to weight management. Consumption of “junk food” that is high in simple sugars and saturated fats has increased dramatically during the past couple decades, particularly in the United States. This has contributed significantly to the obesity epidemic. For many people, relying on willpower and dieting is not sufficient to moderate their consumption of unhealthy “junk food.” The results are dire consequences for their health and well-being.

The invention that is disclosed herein directly addresses this problem by helping a person to selectively reduce consumption and/or absorption of unhealthy food. The invention that is disclosed herein is an innovative technology that can be a key part of a comprehensive system that helps a person to reduce their consumption of unhealthy food, to better manage their energy balance, and to lose weight in a healthy and sustainable manner. In the following sections, we categorize and review the prior art, provide a summary of this invention and its advantages over the prior art, and then provide some detailed examples of how this invention can be embodied to help a person to improve their nutrition and to manage their weight.

### CATEGORIZATION AND REVIEW OF THE PRIOR ART

It can be challenging to classify prior art into discrete categories. This is the certainly the case in the field of energy balance, weight management, and proper nutrition. There are numerous examples of potentially-relevant prior art. However, classification of the prior art into categories, even if imperfect, is an invaluable tool for reviewing the prior art, identifying its limitations, and setting the stage for discussion of the advantages of the invention that is disclosed in subsequent sections. Towards this end, I now identify 50 general categories of prior art and list examples of prior art which appear to be best classified into each category. This categorization and discussion of the prior art helps to identify limitations of the prior art which are corrected by the invention disclosed herein in subsequent sections. The categories of prior art that are most relevant to this invention are marked with an asterisk “*”.

The 50 categories of prior art that I will now discuss are as follows: (1) little or no automated measurement of food consumption, (2) consumed manufactured compound or specifically-isolated natural substance, (3) substance sprinkled on food, (4) manually-ingested spray or pulse, (5) substance-emitting lipstick or toothpaste, (6) substance-emitting adhesive patch in the mouth, (7) dissolving film in mouth, (8) tablet or gum in mouth, (9) intraoral drug delivery, (10*) motion guided or directed pill, (11) general implanted drug pump, (12) food purchasing monitoring or modification, (13) food scale, (14) portion size control, (15) mouth size or function modification, (16*) chewing and swallowing monitoring, (17) hand and/or arm motion monitoring and modification (wrist), (18) hand and/or arm motion monitoring and modification (utensil), (19) utensil with sensor other than motion sensor, (20) other modification of eating speed, (21) photo identification of food (bar code or other packaging-based code), (22) photo identification of food (manual picture taking and identification), (23) photo identification of food (manual picture taking and automated identification), (24*) photo identification of food (automated picture taking and identification), (25) gastric band, (26*) gastric band with sensor, (27*) gastrointestinal (GI) bypass and tissue plication, (28) pumping food out of the stomach through an intra-abdominal pathway, (29) gastric tube, (30) enzyme flow modification, (31) gastrointestinal (GI) volume or pressure or flow modification, (32*) gastrointestinal (GI) volume or pressure or flow modification (with drug), (33) gastrointestinal (GI) sleeve or liner, (34) gastrointestinal (GI) sleeve or liner (with drug), (35*) electrical stimulation (general), (36*) electrical stimulation (with glucose sensor), (37*) electrical stimulation (with general sensor), (38*) electrical stimulation (with taste modification), (39*) electrical stimulation (with drug), (40*) electrical stimulation (with drug and sensor), (41) salivation stimulation, (42*) general sensor (glucose), (43*) general sensor (electromagnetic), (44*) general sensor (chemical), (45*) general sensor (microwave), (46*) sensor (intraoral), (47*) sensor (general), (48) blood analysis, (49) general energy balance feedback, and (50) miscellaneous energy balance related.

### 1. Little (or No) Automatic Measurement of Food Consumption

This category includes prior art with little (or no) automatic measurement of food consumption. The vast majority of art in this category requires a person to take specific action (apart from the actual act of eating) in order to record food consumption. For many years, people did this using pencil and paper. Now they can do it with computer assistance (such as an application on a mobile electronic device), but even the computer-assisted methods in this category still rely on specific human action to record food consumption.

Interfaces for the human action required to record food consumption can include: touch screen; voice and/or speech recognition; keyboard, keypad, or buttons; and mouse, trackball, or touchpad. Gesture recognition may become a more popular interface in future years. Devices comprising art in this category can be worn on a person (e.g. a wrist-mounted band or necklace), carried by a person (e.g. a mobile phone or electronic tablet), or stationary (e.g. a desktop computer). Some wrist-mounted bands and food-serving utensils that do not explicitly track caloric intake are nonetheless included in this category because of their innovative measurement of caloric output and their general relevance to energy balance.

Recent art in this category makes manual recording of food consumption easier with computer-assisted features such as menu-driven user interfaces and voice recognition. These can definitely make it easier for someone to associate specific nutrients or calorie amounts with specific common foods through the use of a food-nutrient database. However, even recent art in this category still requires specific action by a person associated with each eating event apart from the actual act of eating. They offer little (or no) automatic monitoring of food consumption. If a person does not record each food consumption event, then such a device is unaware that food has been consumed. Long-term compliance with manual food logs is notoriously low. People tend to under-estimate calories consumed (especially for unstructured snacking). The accuracy of caloric intake monitoring with art in this category still depends largely, or entirely, on the voluntary compliance of the person whose actions are needed to manually record food consumption. Also, even if food consumption is properly recorded, the success of such art in actually modifying food consumption further depends on the effectiveness of its behavioral modification methods.

Examples of prior art that appear to be best classified in this category include: U.S. Pat. No. 4,100,401 (Jul. 11, 1978 Tutt et al.) “Calorie Calculator-Chronometer”, U.S. Pat. No. 4,212,079 (Jul. 8, 1980 Segar et al.) “Electronic Calorie Counter”, U.S. Pat. No. 4,218,611 (Aug. 19, 1980 Cannon) “Method and Apparatus for Controlling Eating Behavior”, U.S. Pat. No. 4,221,959 (Sep. 9, 1980 Sessler) “Checking Device for Checking the Food Intake”, U.S. Pat. No. 4,310,316 (Jan. 12, 1982 Thomann) “Diet Control Apparatus”, U.S. Pat. No. 4,321,674 (Mar. 23, 1982 Krames et al.) “Nutritional Value Accumulating and Display Device”, U.S. Pat. No. 4,650,218 (Mar. 17, 1987 Hawke) “Method and Apparatus for Controlling Caloric Intake”, U.S. Pat. No. 4,686,624 (Aug. 11, 1987 Blum et al.) “Portable Apparatus for Acquiring and Processing Data Relative to the Dietetics and/or the Health of a Person”, U.S. Pat. No. 4,796,182 (Jan. 3, 1989 Duboff) “Diet Monitor and Display Device”, U.S. Pat. No. 5,173,588 (Dec. 22, 1992 Harrah) “Food Consumption Monitor”, U.S. Pat. No. 5,478,989 (Dec. 26, 1995 Shepley) “Nutritional Information System for Shoppers”, U.S. Pat. No. 5,542,420 (Aug. 6, 1996 Goldman et al.) “Personalized Method and System for Storage, Communication, Analysis, and Processing of Health-Related Data”, U.S. Pat. No. 5,673,691 (Oct. 7, 1997 Abrams et al.) “Apparatus to Control Diet and Weight Using Human Behavior Modification Techniques”, U.S. Pat. No. 5,691,927 (Nov. 25, 1997 Gump) “Nutritional Aid and Method”, U.S. Pat. No. 5,704,350 (Jan. 6, 1998 Williams) “Nutritional Microcomputer and Method”, U.S. Pat. No. 5,729,479 (Mar. 17, 1998 Golan) “Multifunctional Diet Calculator”, U.S. Pat. No. 5,836,312 (Nov. 17, 1998 Moore) “Computer-Assisted System and Method for Adjudging the Effect of Consumable Intakes on Physiological Parameters”, U.S. Pat. No. 5,839,901 (Nov. 24, 1998 Karkanen) “Integrated Weight Loss Control Method”, U.S. Pat. No. 5,841,115 (Nov. 24, 1998 Shepley) “Nutritional Information System for Shoppers”, U.S. Pat. No. 5,890,128 (Mar. 30, 1999 Diaz et al.) “Personalized Hand Held Calorie Computer (ECC)”, U.S. Pat. No. 5,989,188 (Nov. 23, 1999 Birkhoelzer) “Method and Apparatus for Determining the Energy Balance of a Living Subject on the Basis of Energy Used and Nutrition Intake”, U.S. Pat. No. 6,024,281 (Feb. 15, 2000 Shepley) “Nutritional Information System for Shoppers”, and U.S. Pat. No. 6,032,676 (Mar. 7, 2000 Moore) “Method for Correlating Consumable Intakes with Physiological Parameters”.

Examples of prior art that appear to be best classified in this category also include: U.S. Pat. No. 6,040,531 (Mar. 21, 2000 Miller-Kovach) “Process For Controlling Body Weight”, U.S. Pat. No. 6,083,006 (Jul. 4, 2000 Coffman) “Personalized Nutrition Planning”, U.S. Pat. No. 6,095,949 (Aug. 1, 2000 Arai) “Health Management Device”, U.S. Pat. No. 6,336,136 (Jan. 1, 2002 Harris) “Internet Weight Reduction System”, U.S. Pat. No. 6,341,295 (Jan. 22, 2002 Stotler) “Virtual Reality Integrated Caloric Tabulator”, U.S. Pat. No. 6,478,736 (Nov. 12, 2002 Mault) “Integrated Calorie Management System”, U.S. Pat. No. 6,506,152 (Jan. 14, 2003 Lackey et al.) “Caloric Energy Balance Monitor”, U.S. Pat. No. 6,553,386 (Apr. 22, 2003 Alabaster) “System and Method for Computerized Visual Diet Behavior Analysis and Training”, U.S. Pat. No. 6,571,200 (May 27, 2003 Mault) “Monitoring Caloric Expenditure Resulting from Body Activity”, U.S. Pat. No. 6,595,929 (Jul. 22, 2003 Stivoric et al.) “System for Monitoring Health Wellness and Fitness Having a Method and Apparatus for Improved Measurement of Heat Flow”, U.S. Pat. No. 6,605,038 (Aug. 12, 2003 Teller et al.) “System for Monitoring Health, Wellness and Fitness”, U.S. Pat. No. 6,635,015 (Oct. 21, 2003 Sagel) “Body Weight Management System”, U.S. Pat. No. 6,675,041 (Jan. 6, 2004 Dickinson) “Electronic Apparatus and Method for Monitoring Net Calorie Intake”, U.S. Pat. No. 6,694,182 (Feb. 17, 2004 Yamazaki et al.) “Wearable Calorie Calculator”, U.S. Pat. No. 6,745,214 (Jun. 1, 2004 Inoue et al.) “Calorie Control Apparatus with Voice Recognition”, U.S. Pat. No. 6,856,938 (Feb. 15, 2005 Kurtz) “Weight Monitoring Computer”, U.S. Pat. No. 6,878,885 (Apr. 12, 2005 Miller-Kovach) “Process for Controlling Body Weight”, U.S. Pat. No. 6,917,897 (Jul. 12, 2005 Mork) “Food and Exercise Calculator”, U.S. Pat. No. 7,020,508 (Mar. 28, 2006 Stivoric et al.) “Apparatus for Detecting Human Physiological and Contextual Information”, U.S. Pat. No. 7,261,690 (Aug. 28, 2007 Teller et al.) “Apparatus for Monitoring Health, Wellness and Fitness”, U.S. Pat. No. 7,285,090 (Oct. 23, 2007 Stivoric et al.) “Apparatus for Detecting, Receiving, Deriving and Displaying Human Physiological and Contextual Information”, and U.S. Pat. No. 7,361,141 (Apr. 22, 2008 Nissila et al.) “Method and Device for Weight Management of Humans”.

Examples of prior art that appear to be best classified in this category also include: U.S. Pat. No. 7,454,002 (Nov. 18, 2008 Gardner et al.) “Integrating Personal Data Capturing Functionality into a Portable Computing Device and a Wireless Communication Device”, U.S. Pat. No. 7,500,937 (Mar. 10, 2009 Hercules) “Diet Compliance System”, U.S. Pat. No. 7,689,437 (Mar. 30, 2010 Teller et al.) “System for Monitoring Health, Wellness and Fitness”, U.S. Pat. No. 7,857,730 (Dec. 28, 2010 Dugan) “Methods and Apparatus for Monitoring and Encouraging Health and Fitness”, U.S. Pat. No. 7,949,506 (May 24, 2011 Hill et al.) “Method for Determining and Compensating for a Weight Loss Energy Gap”, U.S. Pat. No. 7,959,567 (Jun. 14, 2011 Stivoric et al.) “Device to Enable Quick Entry of Caloric Content”, U.S. Pat. No. 8,073,707 (Dec. 6, 2011 Teller et al.) “System for Detecting Monitoring and Reporting an Individual's Physiological or Contextual Status”, U.S. Pat. No. 8,075,451 (Dec. 13, 2011 Dugan) “Methods and Apparatus for Monitoring and Encouraging Health and Fitness”, U.S. Pat. No. 8,087,937 (Jan. 3, 2012 Peplinski et al.) “System and Method for Monitoring Weight and Nutrition”, U.S. Pat. No. 8,157,731 (Apr. 17, 2012 Teller et al.) “Method and Apparatus for Auto Journaling of Continuous or Discrete Body States Utilizing Physiological and/or Contextual Parameters”, U.S. Pat. No. 8,180,592 (May 15, 2012 Yuen et al.) “Portable Monitoring Devices and Methods of Operating Same”, U.S. Pat. No. 8,311,769 (Nov. 13, 2012 Yuen et al.) “Portable Monitoring Devices and Methods of Operating Same”, and U.S. Pat. No. 8,311,770 (Nov. 13, 2012 Yuen et al.) “Portable Monitoring Devices and Methods of Operating Same”.

Examples of prior art that appear to be best classified in this category also include U.S. patent applications: 20020133378 (Sep. 19, 2002 Mault et al.) “System and Method of Integrated Calorie Management”, 20020156351 (Oct. 24, 2002 Sagel) “Body Weight Management System”, 20030152607 (Aug. 14, 2003 Mault) “Caloric Management System and Method with Voice Recognition”, 20030165799 (Sep. 4, 2003 Bisogno) “Computer Program, Method, and System for Monitoring Nutrition Content of Consumables and for Facilitating Menu Planning”, 20030219513 (Nov. 27, 2003 Gordon) “Personal Nutrition Control Method”, 20040034289 (Feb. 19, 2004 Teller et al.) “System for Monitoring Health, Wellness and Fitness”, 20040133081 (Jul. 8, 2004 Teller et al.) “Method and Apparatus for Auto Journaling of Continuous or Discrete Body States Utilizing Physiological and/or Contextual Parameters”, 20040133081 (Jul. 8, 2004 Teller et al.) “Method and Apparatus for Auto Journaling of Continuous or Discrete Body States Utilizing Physiological and/or Contextual Parameters”, 20040152957 (Aug. 5, 2004 Stivoric et al.) “Apparatus for Detecting, Receiving, Deriving and Displaying Human Physiological and Contextual Information”, 20050004436 (Jan. 6, 2005 Nissila et al.) “Method and Device for Weight Management of Humans”, 20050008994 (Jan. 13, 2005 Bisogno) “Computer Program, Method, and System for Monitoring Nutrition Content of Consumables and for Facilitating Menu Planning”, 20050113650 (May 26, 2005 Pacione et al.) “System for Monitoring and Managing Body Weight and Other Physiological Conditions Including Iterative and Personalized Planning . . . ”, 20050247213 (Nov. 10, 2005 Slilaty) “Method of Identifying Particular Attributes of Food Products Consistent with Consumer Needs and/or Desires”, 20050266385 (Dec. 1, 2005 Bisogno) “Computer Program, Method, and System for Monitoring Nutrition Content of Consumables and for Facilitating Menu Planning”, 20060031102 (Feb. 9, 2006 Teller et al.) “System for Detecting Monitoring and Reporting an Individual's Physiological or Contextual Status”, 20060036395 (Feb. 16, 2006 Shaya et al.) “Method and Apparatus for Measuring and Controlling Food Intake of an Individual”, 20060074716 (Apr. 6, 2006 Tilles et al.) “System and Method for Providing Customized Interactive and Flexible Nutritional Counseling”, and 20060122474 (Jun. 8, 2006 Teller et al.) “Apparatus for Monitoring Health Wellness and Fitness”.

Examples of prior art that appear to be best classified in this category also include U.S. patent applications: 20060264730 (Nov. 23, 2006 Stivoric et al.) “Apparatus for Detecting Human Physiological and Contextual Information”, 20070027366 (Feb. 1, 2007 Osburn) “Device and System for Entering and Monitoring Dietary Data”, 20070089335 (Apr. 26, 2007 Smith et al.) “Nutrient Consumption/Expenditure Planning and Tracking Apparatus System and Method”, 20070106129 (May 10, 2007 Srivathsa et al.) “Dietary Monitoring System for Comprehensive Patient Management”, 20070179355 (Aug. 2, 2007 Rosen) “Mobile Self-Management Compliance and Notification Method, System and Computer Program Product”, 20070208593 (Sep. 6, 2007 Hercules) “Diet Compliance System”, 20080161654 (Jul. 3, 2008 Teller et al.) “Method and Apparatus for Auto Journaling of Body States and Providing Derived Physiological States Utilizing Physiological and/or Contextual Parameter”, 20080161655 (Jul. 3, 2008 Teller et al.) ibid, 20080167536 (Jul. 10, 2008 Teller et al.) ibid, 20080167537 (Jul. 10, 2008 Teller et al.) ibid, 20080167538 (Jul. 10, 2008 Teller et al.) ibid, 20080167539 (Jul. 10, 2008 Teller et al.) ibid, 20080171920 (Jul. 17, 2008 Teller et al.) ibid, 20080171921 (Jul. 17, 2008 Teller et al.) ibid, 20080171922 (Jul. 17, 2008 Teller et al.) ibid, 20080275309 (Nov. 6, 2008 Stivoric et al.) “Input Output Device for Use with Body Monitor”, 20090177068 (Jul. 9, 2009 Stivoric et al.) “Method and Apparatus for Providing Derived Glucose Information Utilizing Physiological and/or Contextual Parameters”, 20090191514 (Jul. 30, 2009 Barnow) “Calorie Counter”, 20100057564 (Mar. 4, 2010 Godsey et al.) “System and Method for Fitness Motivation”, 20100062119 (Mar. 11, 2010 Miller-Kovach) “Processes and Systems for Achieving and Assisting in Improved Nutrition”, 20100062402 (Mar. 11, 2010 Miller-Kovach) “Processes and Systems Using and Producing Food Healthfulness Data Based on Linear Combinations of Nutrients”, 20100079291 (Apr. 1, 2010 Kroll et al.) “Personalized Activity Monitor and Weight Management System”, 20100080875 (Apr. 1, 2010 Miller-Kovach) “Processes and Systems for Achieving and Assisting in Improved Nutrition Based on Food Energy Data and Relative Healthfulness Data”, and 20100228160 (Sep. 9, 2010 Schweizer) “Apparatus for Activity Monitoring”.

Examples of prior art that appear to be best classified in this category also include U.S. patent applications: 20110087137 (Apr. 14, 2011 Hanoun) “Mobile Fitness and Personal Caloric Management System”, 20120031805 (Feb. 9, 2012 Stolarczyk) “Daily Meal Planning System”, 20120072233 (Mar. 22, 2012 Hanlon et al.) “Medical Health Information System for Health Assessment, Weight Management and Meal Planning”, 20120083669 (Apr. 5, 2012 Abujbara) “Personal Nutrition and Wellness Advisor”, 20120083705 (Apr. 5, 2012 Yuen et al.) “Activity Monitoring Systems and Methods of Operating Same”, 20120083714 (Apr. 5, 2012 Yuen et al.) “Activity Monitoring Systems and Methods of Operating Same”, 20120083715 (Apr. 5, 2012 Yuen et al.) “Portable Monitoring Devices and Methods of Operating Same”, 20120083716 (Apr. 5, 2012 Yuen et al.) “Portable Monitoring Devices and Methods of Operating Same”, 20120084053 (Apr. 5, 2012 Yuen et al.) “Portable Monitoring Devices and Methods of Operating Same”, 20120084054 (Apr. 5, 2012 Yuen et al.) “Portable Monitoring Devices and Methods of Operating Same”, 20120096405 (Apr. 19, 2012 Seo) “Apparatus and Method for Diet Management”, 20120126983 (May 24, 2012 Breibart) “Method and Associated Device for Personal Weight Control or Weight Loss”, 20120221495 (Aug. 30, 2012 Landers) “Digital Weight Loss Aid”, 20120226471 (Sep. 6, 2012 Yuen et al.) “Portable Monitoring Devices and Methods of Operating Same”, 20120226472 (Sep. 6, 2012 Yuen et al.) “Portable Monitoring Devices and Methods of Operating Same”, 20120295233 (Nov. 22, 2012 Cooperman) “Computerized System and Method for Monitoring Food Consumption”, 20120316932 (Dec. 13, 2012 Rahman et al.) “Wellness Application for Data-Capable Band”, 20120317167 (Dec. 13, 2012 Rahman et al.) “Wellness Application for Data-Capable Band”, 20130002435 (Jan. 3, 2013 Utter) “Sleep Management Method and Apparatus for a Wellness Application Using Data from a Data-Capable Band”, 20130006063 (Jan. 3, 2013 Wang) “Physiological Condition, Diet and Exercise Plan Recommendation and Management System”, 20130006125 (Jan. 3, 2013 Kroll et al.) “Personalized Activity Monitor and Weight Management System”, and 20130029807 (Jan. 31, 2013 Amsel) “Health Tracking Program”.

### 2. Consumed Manufactured Compound or Specifically-Isolated Natural Substance

Prior art in this category includes manufactured compounds and specifically-isolated natural substances that are either added to food as an ingredient during food preparation or are consumed independently of food consumption in order to modify a person's food consumption. This category includes pharmaceuticals and specific food ingredients that are intended as appetite suppressants. For many years people have been seeking a “magic” pill that can address obesity with good results and tolerable side effects.

There are many examples of prior art in this category and we have only included those which appear to be most relevant. For the purposes of this categorization, we have created a separate subsequent category for substances which a person can sprinkle on food at the time of consumption. We have also included separate categories for inventions whose primary therapeutic modality is a device, but which also emit or elude a drug as a secondary mode of action. The success of art in this category for modifying food consumption depends on the substance's ability to actually modify the person's food consumption without intolerable side effects. Compliance and effectiveness can be problematic, especially if a drug's side effects are very unpleasant.

Examples of prior art that appear to be best classified in this category include: U.S. Pat. No. 4,159,347 (Jun. 26, 1979 Yoshida et al.) “Flavoring with Cyclic Acetals of 2-Methyl-2-Pentenal”, U.S. Pat. No. 4,210,637 (Jul. 1, 1980 Wurtman et al.) “Composition and Method for Suppressing Appetite for Calories as Carbohydrates”, U.S. Pat. No. 4,491,578 (Jan. 1, 1985 Peikin) “Method of Stimulating Satiety in Mammals”, U.S. Pat. No. 4,497,798 (Feb. 5, 1985 Lambert) “Appetite Suppressant”, U.S. Pat. No. 4,689,235 (Aug. 25, 1987 Barnes et al.) “Encapsulation Matrix Composition and Encapsulate Containing Same”, U.S. Pat. No. 4,740,365 (Apr. 26, 1988 Yukimatsu et al.) “Sustained-Release Preparation Applicable to Mucous Membrane in Oral Cavity”, U.S. Pat. No. 5,013,716 (May 7, 1991 Cherukuri et al.) “Unpleasant Taste Masking Compositions and Methods for Preparing Same”, U.S. Pat. No. 5,290,808 (Mar. 1, 1994 Sofia) “Method to Control the Intake of Food”, U.S. Pat. No. 5,405,641 (Apr. 11, 1995 Kurihara et al.) “Taste-Modification Composition and Method for Stabilizing Taste-Modifier”, U.S. Pat. No. 5,472,685 (Dec. 5, 1995 Gaffar) “Antiplaque Oral Compositions”, U.S. Pat. No. 5,605,698 (Feb. 25, 1997 Ueno) “Oral Composition”, U.S. Pat. No. 5,858,967 (Jan. 12, 1999 Weigle et al.) “Appetite Supression Factor and Related Methods”, U.S. Pat. No. 6,123,980 (Sep. 26, 2000 Pearson et al.) “Preparing Granulated Sugar Blends and Products”, U.S. Pat. No. 6,207,638 (Mar. 27, 2001 Portman) “Nutritional Intervention Composition for Enhancing and Extending Satiety”, U.S. Pat. No. 6,224,873 (May 1, 2001 Jones) “Regulation of Appetite Body Weight and Athletic Function with Materials Derived from Citrus Varieties”, U.S. Pat. No. 6,235,274 (May 22, 2001 Lou et al.) “Microparticles Which Controllably Release Olfactorily Active Substances Methods of Using Same and Processes for Preparing Same”, U.S. Pat. No. 6,248,390 (Jun. 19, 2001 Stillman) “Fiber-Water: Water Containing Soluble Fiber”, U.S. Pat. No. 6,319,523 (Nov. 20, 2001 Zhou) “Composition and Method for Inhibiting Oral Bacteria”, U.S. Pat. No. 6,376,657 (Apr. 23, 2002 Van Heerden et al.) “Pharmaceutical Compositions Having Appetite Suppressant Activity”, U.S. Pat. No. 6,413,545 (Jul. 2, 2002 Alviar et al.) “Diet Composition and Method of Weight Management”, U.S. Pat. No. 6,610,277 (Aug. 26, 2003 Zuckerman) “Appetite Suppressant Toothpaste”, and U.S. Pat. No. 6,861,405 (Mar. 1, 2005 Desir et al.) “Compositions and Methods Relating to Glucose Metabolism, Weight Control, and Food Intake”.

Examples of prior art that appear to be best classified in this category also include: U.S. Pat. No. 6,942,848 (Sep. 13, 2005 Nelson et al.) “Cyclodextrins in Dental Products”, U.S. Pat. No. 7,025,984 (Apr. 11, 2006 Jandacek et al.) “Compositions and Methods for Body Weight Management”, U.S. Pat. No. 7,115,297 (Oct. 3, 2006 Stillman) “Nutritionally Fortified Liquid Composition with Added Value Delivery Systems/Elements/Additives”, U.S. Pat. No. 7,138,107 (Nov. 21, 2006 Adams et al) “Inhibition of Olfactory Neurosensory Function to Treat Eating Disorders and Obesity”, U.S. Pat. No. 7,229,658 (Jun. 12, 2007 Inoue et al.) “Compositions Containing Sucralose and Application Thereof”, U.S. Pat. No. 7,238,380 (Jul. 3, 2007 Stillman) “Water Containing Soluble Fiber”, U.S. Pat. No. 7,276,229 (Oct. 2, 2007 Baker et al.) “Oral Compositions”, U.S. Pat. No. 7,402,400 (Jul. 22, 2008 Zuker et al.) “Mammalian Sweet Taste Receptors”, U.S. Pat. No. 7,524,877 (Apr. 28, 2009 Rosenfeld et al.) “Compounds for Use in Weight Loss and Appetite Suppression in Humans”, U.S. Pat. No. 7,541,356 (Jun. 2, 2009 Rosenfeld et al.) “Compounds for Use in Weight Loss and Appetite Suppression in Humans”, U.S. Pat. No. 7,632,517 (Dec. 15, 2009 Dugger et al.) “Buccal Polar and Non-Polar Spray Containing Zolpidem”, U.S. Pat. No. 7,851,005 (Dec. 14, 2010 Bingley et al.) “Taste Potentiator Compositions and Beverages Containing Same”, U.S. Pat. No. 7,851,006 (Dec. 14, 2010 Bingley et al.) “Taste Potentiator Compositions and Beverages Containing Same”, U.S. Pat. No. 7,879,376 (Feb. 1, 2011 Boghani et al.) “Taste Potentiator Compositions and Edible Confectionery and Chewing Gum Products Containing Same”, U.S. Pat. No. 7,977,060 (Jul. 12, 2011 Zuker et al.) “Mammalian Sweet Taste Receptors”, U.S. Pat. No. 8,119,359 (Feb. 21, 2012 Adler et al.) “Methods of Identifying Sweet Taste Modulators”, U.S. Pat. No. 8,143,215 (Mar. 27, 2012 Hirsch) “Method of Promoting Weight Loss”, U.S. Pat. No. 8,198,048 (Jun. 12, 2012 Zuker et al.) “Mammalian Sweet Taste Receptors”, U.S. Pat. No. 8,217,001 (Jul. 10, 2012 Cowley et al.) “Modification of Feeding Behavior”, U.S. Pat. No. 8,236,285 (Aug. 7, 2012 Dugger et al.) “Buccal, Polar and Non-Polar Spray Containing Zolpidem”, and U.S. Pat. No. 8,287,898 (Oct. 16, 2012 Jandacek et al.) “Compositions and Methods for Body Weight Management”.

Examples of prior art that appear to be best classified in this category also include U.S. patent applications: 20020187204 (Dec. 12, 2002 Alviar et al.) “Diet Composition and Method of Weight Management”, 20030113310 (Jun. 19, 2003 Van Laere et al.) “Method for the Treatment of Obesity, Overweight and Fluctuations in Blood Insuline and/or Glucose Levels”, 20040071801 (Apr. 15, 2004 Edell et al.) “Herbal Formulation of Gymnema Sylvestre as a Dietary Aid”, 20040156920 (Aug. 12, 2004 Kane) “Extracts From Plant and Non-Plant Biomass and Uses Thereof”, 20040192760 (Sep. 30, 2004 Whittle et al.) “Pharmaceutical Formulations”, 20040247702 (Dec. 9, 2004 Rajendran et al.) “Caralluma Extract Products and Processes for Making the Same”, 20050053555 (Mar. 10, 2005 Pederson) “Appetite Control Compositions and Methods of Use”, 20060105068 (May 18, 2006 Fleischner) “Dietary Supplement Formulations Containing Hoodia Gordonii”, 20060193795 (Aug. 31, 2006 Zuckerman) “Appetite Suppressant Mouth Spray”, 20070104805 (May 10, 2007 Udell) “Compositions of Hoodia Gordonii and Pinolenic Acid Derivatives”, 20070160735 (Jul. 12, 2007 Stillman) “Water Containing Soluble Fiber”, 20070196436 (Aug. 23, 2007 Abrahams et al.) “Process for Preparing an Edible Composition Comprising Steroidal Glycosides”, 20080014327 (Jan. 17, 2008 Stillman) “Water Containing Soluble Fiber”, 20080102143 (May 1, 2008 Freis et al.) “Uses for the Extract of a Plant of the Family Asclepiadaceae”, 20080138447 (Jun. 12, 2008 Riggins et al.) “Method for Administering Appetite Suppressant and Composition Thereof”, 20080152705 (Jun. 26, 2008 Udell et al.) “Corosolic Acid Formulation and Its Application for Weight-Loss Management and Blood Sugar Balance”, and 20080255093 (Oct. 16, 2008 Tam et al.) “Compositions and Methods for Treating Obesity and Related Disorders”.

Examples of prior art that appear to be best classified in this category also include U.S. patent applications: 20100098783 (Apr. 22, 2010 Sommerfeld et al.) “Appetite Suppressant Composition”, 20100215584 (Aug. 26, 2010 Passe) “Compositions and Methods of Modulating the Taste and Smell Receptors and Screening Methods Therefore”, 20100267643 (Oct. 21, 2010 Baron et al.) “Chemosensory Receptor Ligand-Based Therapies”, 20100316768 (Dec. 16, 2010 Stillman) “Nutritionally Fortified Liquid Composition with Added Value Delivery Systems/Elements/Additives”, 20110065660 (Mar. 17, 2011 Baron et al.) “Chemosensory Receptor Ligand-Based Therapies”, 20110082407 (Apr. 7, 2011 Aronne) “Combination Therapies for the Treatment of Obesity”, 20110104336 (May 5, 2011 Stillman) “Water Containing Soluble Fiber”, 20110136909 (Jun. 9, 2011 Imada et al.) “Method for Suppressing Excessive Appetite”, 20110166065 (Jul. 7, 2011 Bhanot et al.) “Modulation Of Glucose-6-Phosphatase Translocase Expression”, 20110224155 (Sep. 15, 2011 Tachdjian et al.) “Modulation of Chemosensory Receptors and Ligands Associated Therewith”, 20110230502 (Sep. 22, 2011 Tachdjian et al.) “Modulation of Chemosensory Receptors and Ligands Associated Therewith”, 20110244514 (Oct. 6, 2011 Zuker et al.) “Mammalian Sweet Taste Receptors”, 20120040893 (Feb. 16, 2012 Cowley et al.) “Modification of Feeding Behaviour”, 20120094942 (Apr. 19, 2012 Baron et al.) “Chemosensory Receptor Ligand-Based Therapies”, 20120115778 (May 10, 2012 Karsenty et al.) “Methods of Suppressing Appetite by the Administration of Antagonists of the Serotonin HTR1a or HTR2b Receptors or Inhibitors of TPH2”, 20120157409 (Jun. 21, 2012 Cherkassky) “Appetite Suppressant Product and Method”, 20120177730 (Jul. 12, 2012 Baron et al.) “Chemosensory Receptor Ligand-Based Therapies”, and 20120208748 (Aug. 16, 2012 Chen et al.) “Peptide Compositions and Methods for Treating Patients”. Examples of prior art that appear to be best classified in this category also include EP 1685834 “Use of Pinolenic Acid for the Treatment Of Obesity” and EP 2072048 “Use of Pinolenic Acid for the Treatment Of Obesity”.

### 3. Substance Sprinkled on Food

Prior art in this category includes manufactured and specifically-isolated substances or compounds that a person voluntarily adds to their food slightly before or during food consumption in order to modify their food consumption. For example, this category includes substances that a person sprinkles on their food with the intent of suppressing their appetite. In various examples, such a substance can change the flavor, smell, or appearance of food with the intent of dampening a person's appetite.

The success of art in this category in modifying food consumption depends on the ability of the sprinkled substance to actually modify the person's food consumption and the consistency with which the person regularly sprinkles the substance on food each time they eat. This can be problematic, especially if the substance makes food taste less appealing or if a specific food has a surface to which the sprinkled substance does not adhere. Also, if a person does not have enough willpower and discipline to avoid eating unhealthy food in the first place, then it is not clear that this person would have enough willpower and discipline to always sprinkle an appetite-suppressing additive on their food each time that they eat.

Examples of prior art that appear to be best classified in this category include: U.S. Pat. No. 5,603,971 (Feb. 18, 1997 Porzio et al.) “Encapsulation Compositions”, U.S. Pat. No. 6,112,749 (Sep. 5, 2000 Hall et al.) “Flavor Dot Odorizer and Method”, U.S. Pat. No. 6,902,751 (Jun. 7, 2005 Schleifenbaum et al.) “Encapsulated Flavorings”, U.S. Pat. No. 7,727,546 (Jun. 1, 2010 Moneymaker et al.) “Nutrient System for Individualized Responsive Dosing Regimens”, U.S. Pat. No. 7,820,208 (Oct. 26, 2010 Hirsch) “Method of Assaying Satiety Enhancing Tastants (Alan Hirsch)”, U.S. Pat. No. 8,143,062 (Mar. 27, 2012 Hirsch) “Method and Composition for Enhancing Weight Loss”, and U.S. Pat. No. 8,143,215 (Mar. 27, 2012 Hirsch) “Method of Promoting Weight Loss”; and U.S. patent applications 20040231299 (Nov. 25, 2004 Yakushigawa et al.) “Flavoring System and Method”, 20080075813 (Mar. 27, 2008 Smith et al.) “Seasoning and Method for Enhancing and Potentiating Food Flavor Utilizing Microencapsulation While Reducing Dietary Sodium Intake”, 20090123380 (May 14, 2009 Hirsch) “Method of Assaying Satiety Enhancing Tastants (Alan Hirsch)”, 20090123524 (May 14, 2009 Hirsch) “Packaged Satiety Enhancing Composition (Alan Hirsch)”, 20090123579 (May 14, 2009 Hirsch) “Method of Promoting Weight Loss (Alan Hirsh)”, 20090214445 (Aug. 27, 2009 Boghani et al.) “Delivery Systems for Managing Release of Functional Ingredients in an Edible Composition”, and 20120058217 (Mar. 8, 2012 Patty) “Taste Deterrent and Diet Method”.

### 4. Manually-Administered Spray or Pulse

This category of prior art includes oral and nasal sprays, mists, and pulses that contain a consumption-modifying substance. As was the case with art involving a sprinkled food additive, the success of art in this category depends on the ability of the sprayed substance to actually modify a person's food consumption and the regularity with which the person sprays the substance into their mouth or nose every time that they eat. In an example, a sprayed substance can be absorbed into tissue for a systemic (pharmacologic) appetite-suppressant effect. In another example, a sprayed substance can be released into a person's oral cavity or nasal cavities for a localized anesthetic effect. In an example, this substance can mask or block the taste or smell of food.

In order for this approach to work, a person must exercise consistent voluntary compliance in spraying the substance into their mouth or nose prior to consumption of (selected types of) food. However, if a person does not have enough willpower and discipline to avoid eating unhealthy food in the first place, then it is not clear that this person would have enough willpower and discipline to consistently spray something into their nose or mouth before every meal or snack.

Examples of prior art that appear to be best classified in this category include: U.S. Pat. No. 4,935,225 (Jun. 19, 1990 Curtis et al.) “Appetite Suppresant Dentifrice”, U.S. Pat. No. 5,284,132 (Feb. 8, 1994 Geier) “Device for the Transnasal or Oral Administration of Drugs or the Like”, U.S. Pat. No. 5,456,677 (Oct. 10, 1995 Spector) “Method for Oral Spray Administration of Caffeine”, U.S. Pat. No. 6,715,485 (Apr. 6, 2004 Djupesland) “Nasal Delivery Device”, U.S. Pat. No. 7,935,065 (May 3, 2011 Martin et al.) “Oral Device”, and patent application 20050037031 (Feb. 17, 2005 Jackson) “Methods for Diet and Weight Control by Altering the Senses of Smell and Taste”.

### 5. Substance-Emitting Lipstick or Toothpaste

This category of prior art includes lipstick or toothpaste that releases a consumption-modifying substance. In order to be effective, the lipstick or toothpaste must release a genuinely consumption-modifying substance in sufficient amounts over a long-enough duration to affect food consumption. If it only releases the substance for a short time or tapers off rapidly, then the lipstick or toothpaste must be applied frequently which relies heavily on the person's voluntary compliance. If it releases the substance for a long time, then the prior art does not disclose how this approach would enable selective modification of unhealthy food consumption; it would affect consumption of healthy foods as well as unhealthy foods. In order for this approach to be effective: the substance in the lipstick or toothpaste must really reduce food consumption when used; the substance must be released from the lipstick or toothpaste in sufficient quantity, and over a sufficient duration, to be effective; and the person must have consistent voluntary compliance in using the lipstick or toothpaste. Also, many people do not wear lipstick. For these reasons, art in this category is limited for consistent modification of food consumption.

Examples of prior art that appear to be best classified in this category include: U.S. Pat. No. 6,485,710 (Nov. 26, 2002 Zuckerman) “Appetite Suppressant Toothpaste” and U.S. Pat. No. 7,247,323 (Jul. 24, 2007 George et al.) “Delivery System for Appetite Suppressant”; and U.S. patent applications 20030095936 (May 22, 2003 Light) “Lip Gloss Composition”, 20070042058 (Feb. 22, 2007 George et al.) “Delivery System for Appetite Suppressant”, and 20100135945 (Jun. 3, 2010 Murdock et al.) “Gymnema-Containing Lip Balm Compositions and Associated Method”.

### 6. Substance-Emitting Adhesive Patch in the Mouth

Prior art in this category includes temporary substance-emitting patches that a person attaches (e.g. through adhesion) within their oral cavity in order to modify their food consumption. In various examples, such a patch can be attached to a person's upper palate or teeth. In an example, this substance can be absorbed into tissue (such as through mucosal delivery) to cause a systemic (pharmacological) appetite-suppressant effect. In an example, this substance can be released into the person's oral cavity or nasal cavity to cause a localized anesthetic effect. The intent is to reduce a person's appetite by gradual emission of an appetite-suppressing substance.

The success of this approach depends on: whether the person regularly uses and replaces the patch, whether the patch emits the substance for a sufficiently long time and in a sufficiently consistent dosage to affect all of a person's meals throughout the day, and whether the substance actually reduces the person's appetite even when consistently emitted. If the effect of the patch lasts for a short time, then the patch must be replaced frequently, which requires high voluntary compliance by the person. If the effect lasts for a long time, then the prior art does not disclose how this approach would enable selective consumption modification (allowing healthy food but discouraging unhealthy food). All of these factors make this approach problematic.

Examples of prior art that appear to be best classified in this category include: U.S. Pat. No. 3,972,995 (Aug. 3, 1976 Tsuk et al.) “Dosage Form”, U.S. Pat. No. 4,059,686 (Nov. 22, 1977 Tanaka et al.) “Pharmaceutical Preparation for Oral Cavity Administration”, U.S. Pat. No. 4,292,299 (Sep. 29, 1981 Suzuki et al.) “Slow-Releasing Medical Preparation to be Administered by Adhering to a Wet Mucous Surface”, U.S. Pat. No. 4,615,697 (Oct. 7, 1986 Robinson) “Bioadhesive Compositions and Methods of Treatment Therewith”, U.S. Pat. No. 4,764,378 (Aug. 16, 1988 Keith et al.) “Buccal Drug Dosage Form”, U.S. Pat. No. 6,387,408 (May 14, 2002 Illum et al.) “Adhesive Drug Delivery Composition”, U.S. Pat. No. 6,488,953 (Dec. 3, 2002 Halliday et al.) “Oral Transmucosal Delivery”, and U.S. Pat. No. 8,173,113 (May 8, 2012 Scholz et al.) “Bioadhesive Composition and Patch”; and U.S. patent applications 20040109886 (Jun. 10, 2004 Rigby) “Methods and Apparatus for Transdermal Delivery of Abusable Drugs with a Deterrent Agent”, 20070104783 (May 10, 2007 Domb et al.) “Double-Layered Absorbable Solid Compositions for the Topical Treatment of Oral Mucosal Disorders”, 20090130178 (May 21, 2009 Oronsky et al.) “Formulation for Decreasing Tobacco, Alcohol, Drug or Food Consumption”, and 20120015021 (Jan. 19, 2012 Mizrahi et al.) “Anti-Appetite Adhesive Compositions”.

### 7. Dissolving Film in Mouth

This category of prior art includes dissolvable films which a person inserts into their mouth and which slowly release a consumption-modifying substance. Unlike art in the prior category, these films are not attached to tissue within a person's oral cavity. Since inserting and ingesting the film can interfere with the process of food consumption, a person must have sufficient willpower and discipline to insert the film in advance of eating. Further, if the substance in the mouth is diluted by food consumption, then the person may have to insert a dissolvable film multiple times during the same meal.

In order for this approach to work, the person must exercise consistent voluntary compliance in inserting the film into their mouth before eating (selected types of) food. However, if a person does not have enough willpower and discipline to avoid eating unhealthy food in the first place, then it is not clear that this person would have enough willpower and discipline to consistently insert a dissolvable film into their mouth before each snack or meal.

Examples of prior art that appear to be best classified in this category include: U.S. Pat. No. 6,419,903 (Jul. 16, 2002 Xu et al.) “Breath Freshening Film” and U.S. Pat. No. 7,972,618 (Jul. 5, 2011 Fuisz et al.) “Edible Water-Soluble Film Containing a Foam Reducing Flavoring Agent”; and patent application 20040131661 (Jul. 8, 2004 Auffret et al.) “Process for Making Orally Consumable Dosage Forms”.

### 8. Tablet or Gum in Mouth

This category of prior art includes tablets, lozenges, and chewing gum that are inserted into the mouth and slowly release a consumption-modifying substance. Since inserting and ingesting a tablet, lozenge, or chewing gum can interfere with the process of food consumption, the person must have sufficient willpower and discipline to insert the tablet, lozenge, or chewing gum well in advance of eating. Further, if the substance in the mouth is diluted by food consumption, then the person may have to insert a tablet, lozenge, or chewing gum multiple times during the same meal.

In order for this approach to work, the person must exercise consistent voluntary compliance in inserting the tablet, lozenge, or chewing gum into their mouth before eating (selected types of) food. However, if a person does not have enough willpower and discipline to avoid eating unhealthy food in the first place, then it is not clear that this person would have enough willpower and discipline to consistently pop a tablet, lozenge, or chewing gum into their mouth before each snack or meal.

Examples of prior art that appear to be best classified in this category include: U.S. Pat. No. 3,856,942 (Dec. 24, 1974 Murphy) “Appetite Control Composition”, U.S. Pat. No. 3,911,099 (Oct. 7, 1975 Defoney et al.) “Long-Acting Articles for Oral Delivery and Process”, U.S. Pat. No. 4,039,653 (Aug. 2, 1977 Defoney et al.) “Long-Acting Articles for Oral Delivery and Process”, U.S. Pat. No. 4,822,597 (Apr. 18, 1989 Faust et al.) “Anesthetic-Containing Chewing Gum Compositions”, U.S. Pat. No. 5,942,244 (Aug. 24, 1999 Friedman et al.) “Local Oral Herbal Slow Release Tablets”, U.S. Pat. No. 6,183,775 (Feb. 6, 2001 Ventouras) “Buccal Delivery System”, U.S. Pat. No. 6,280,761 (Aug. 28, 2001 Santus) “Nicotine Lozenge (Santus)”, U.S. Pat. No. 6,893,654 (May 17, 2005 Pinney et al.) “Two-Stage Transmucosal Medicine Delivery System for Symptom Relief”, U.S. Pat. No. 6,949,264 (Sep. 27, 2005 Mcgrew et al.) “Nutraceuticals or Nutritional Supplements and Method of Making”, U.S. Pat. No. 7,851,000 (Dec. 14, 2010 Boghani et al.) “Taste Potentiator Compositions and Edible Confectionery and Chewing Gum Products Containing Same”, and U.S. Pat. No. 8,236,348 (Aug. 7, 2012 Gin et al.) “Long-Lasting, Flavored Dosage Forms for Sustained Release of Beneficial Agents within the Mouth”; and U.S. patent applications 20040151771 (Aug. 5, 2004 Gin et al.) “Long-Lasting, Flavored Dosage Forms for Sustained Release of Beneficial Agents Within the Mouth”, 20040247669 (Dec. 9, 2004 Gin et al.) “Long-Lasting Flavored Dosage Forms for Sustained Release of Beneficial Agents within the Mouth”, 20050112149 (May 26, 2005 Belote et al.) “Single-Dose Taste Inhibitor Units”, 20070048369 (Mar. 1, 2007 Foreman et al.) “Mucosal Delivery Tablet”, 20090081291 (Mar. 26, 2009 Gin et al.) “Sustained Release Dosage Forms for Delivery of Agents to an Oral Cavity of a User”, and 20120195954 (Aug. 2, 2012 Maynard) “Method of Reducing Appetite”.

### 9. Intraoral Drug Delivery

Prior art in this category includes pharmaceutical compounds that are delivered intra-orally. In an example, a compound can be delivered locally (e.g. by injection) in order to selectively target intraoral tissue. In another example, a compound can be delivered systemically via mucosal absorption. This approach depends on the ability of the pharmaceutical compound to actually reduce a person's appetite and on patient compliance with intra-oral drug administration.

Examples of prior art that appear to be best classified in this category include: U.S. Pat. No. 5,194,003 (Mar. 16, 1993 Garay et al.) “Removable Device for Delivering Beneficial Agents Orally” and U.S. Pat. No. 8,181,655 (May 22, 2012 Bardach et al.) “Therapeutic and Protective Dental Device Useful as an Intra-Oral Delivery System”; and patent application 20080044797 (Feb. 21, 2008 Bardach et al.) “Inserts for Use with Oral Appliances”.

### 10. Motion Guided or Directed Pill

Prior art in this category includes “smart pills” whose movement, placement, attachment, and/or activation within specific body structures can be remotely guided and controlled. In an example, such pills can be guided to a particular location along a person's gastrointestinal tract and then activated when they reach this location. Such activation can include remote-controlled attachment to specific body tissue and/or remote-controlled localized emission of a pharmaceutical compound. In an example, local intragastric drug delivery can be more targeted and effective than systemic drug delivery.

Examples of prior art that appear to be best classified in this category include: U.S. Pat. No. 8,109,920 (Feb. 7, 2012 Boyden et al.) “Medical or Veterinary Digestive Tract Utilization Systems and Methods”, U.S. Pat. No. 8,219,171 (Jul. 10, 2012 Benoist) “Delivery Device for Implantable Monitor”, U.S. Pat. No. 8,303,573 (Nov. 6, 2012 Boyden et al.) “Medical or Veterinary Digestive Tract Utilization Systems and Methods”, and U.S. Pat. No. 8,333,754 (Dec. 18, 2012 Boyden et al.) “Medical or Veterinary Digestive Tract Utilization Systems and Methods”; and U.S. patent applications 20110160129 (Jun. 30, 2011 Imran) “Therapeutic Agent Preparations for Delivery Into a Lumen of the Intestinal Tract Using a Swallowable Drug Delivery Device”, 20110160699 (Jun. 30, 2011 Imran) “Swallowable Drug Delivery Device and Methods of Drug Delivery”, 20120010590 (Jan. 12, 2012 Imran) “Swallowable Drug Delivery Device and Method of Delivery”, 20120165792 (Jun. 28, 2012 Ortiz et al.) “Pill Catchers”, 20120165793 (Jun. 28, 2012 Ortiz et al.) “Pill Catchers”, 20120165794 (Jun. 28, 2012 Ortiz et al.) “Pill Catchers”, and 20120165796 (Jun. 28, 2012 Ortiz et al.) “Pill Catchers”.

### 11. General Implanted Drug Pump

This category of prior art includes implantable drug pumps that are used to achieve a consumption-modifying effect. Not all implantable drug pumps are reviewed here, only those which are particularly relevant to modification of food consumption and related metabolic processes. In an example, an implantable pump can pump a drug into a location along the person's digestive tract. In an example, an implantable drug pump can pump a pharmaceutical agent into a person's brain. In an example, an implantable pump can deliver a pharmaceutical agent into a person's blood stream. For implanted medical devices for which drug delivery appears to be the secondary mode of action, we have included such art in separate categories which follow that are primarily identified by their primary (non-drug device) mode of action.

It is not clear from the prior art how such drug pumps can be selectively used to allow consumption of healthy food, but discourage consumption of unhealthy food. Also, the prior art does not disclose how such devices could be used to allow moderate consumption, but limit excess consumption, of certain foods. Prior art in this category is much less dependent on patient compliance than art in many of the previous categories, but still critically depends on the effectiveness of a drug in modifying food consumption and/or absorption without intolerable side effects.

Examples of prior art that appear to be best classified in this category include: U.S. Pat. No. 4,925,446 (May 15, 1990 Garay et al.) “Removable Inflatable Intragastrointestinal Device for Delivering Beneficial Agents”, U.S. Pat. No. 5,011,472 (Apr. 30, 1991 Aebischer et al.) “Implantable Delivery System for Biological Factors”, U.S. Pat. No. 5,318,519 (Jun. 7, 1994 Wilk) “Method and Apparatus for Supplying Nutrition”, U.S. Pat. No. 5,643,207 (Jul. 1, 1997 Rise) “Implantable Techniques for Infusing a Therapeutic Agent with Endogenous Bodily Fluid”, U.S. Pat. No. 5,730,722 (Mar. 24, 1998 Wilk) “Method and Apparatus for Supplying a Medical Treatment Composition to a Patient”, U.S. Pat. No. 7,043,295 (May 9, 2006 Starkebaum) “Methods and Apparatus for Delivering a Drug Influencing Appetite for Treatment of Eating Disorders”, U.S. Pat. No. 7,108,680 (Sep. 19, 2006 Rohr et al.) “Closed-Loop Drug Delivery System”, U.S. Pat. No. 7,790,671 (Sep. 7, 2010 Stojanovic-Susulic et al.) “Implantable Pump for Protein Delivery for Obesity Control by Drug Infusion into the Brain”, U.S. Pat. No. 8,066,689 (Nov. 29, 2011 Mitelberg et al.) “Methods and Systems for Submucosal Implantation of a Device for Diagnosis and Treatment with a Therapeutic Agent”, and U.S. Pat. No. 8,252,744 (Aug. 28, 2012 Stojanovic-Susulic et al.) “Implantable Pump for Protein Delivery for Obesity Control by Drug Infusion into the Brain”; and U.S. patent applications 20030171711 (Sep. 11, 2003 Rohr et al.) “Closed-Loop Drug Delivery System”, 20050038415 (Feb. 17, 2005 Rohr et al.) “Method and Apparatus for the Treatment of Obesity”, 20050096514 (May 5, 2005 Starkebaum) “Gastric Activity Notification”, 20070082843 (Apr. 12, 2007 Stojanovic-Susulic et al.) “Implantable Pump for Protein Delivery for Obesity Control by Drug Infusion into the Brain”, 20100145301 (Jun. 10, 2010 Magal) “Spray Administration of Compositions Including Active Agents Such as Peptides to the Gastrointestinal Tract”, and 20120071812 (Mar. 22, 2012 Mitelberg et al.) “Methods and Systems for Submucosal Implantation of a Device for Diagnosis and Treatment with a Therapeutic Agent”.

Examples of prior art that appear to be best classified in this category also include EP 1504778 “Implantable Pump for the Treatment of Obesity”, WO 2002085428 (“Implantable Osmotic Pump”), and WO 2003004034 (“Method for Inducing Analgesia Comprising Administration Alternatively of an Opioid Receptor Agonist and an Opioid Receptor Like Receptor 1 Agonist . . . ”).

### 12. Food Purchasing Monitoring

Prior art in this category includes devices and methods that monitor what types of food a person purchases at the point of sale. Although there can be overlap, in some respects most art in this category is based on information technology, not biomedical technology. It is relatively easy to track food purchase transactions at a given store or with a given credit card. It can also be relatively easy to record the many items in a store that are marked with a bar code (or other type of product identifier).

However, this approach depends on two large assumptions. First, it assumes that a person buys everything that they eat at participating locations or with a selected card. This is violated if a person buys food at a non-participating location or eats food that someone else has bought. Second, it assumes that a person eats everything that they buy. This is violated if the person buys food for others (such as their family) and/or does not eat all the food that they buy. Also, timing differences between when a person buys food and when they eat that food can confound analysis of the relationship between food consumption and achievement of weight management objectives.

Examples of prior art that appear to be best classified in this category include: U.S. Pat. No. 5,412,564 (May 2, 1995 Ecer) “System and Method for Diet Control”, U.S. Pat. No. 7,769,635 (Aug. 3, 2010 Simons-Nikolova) “Weight Management System with Simple Data Input”, and U.S. Pat. No. 7,999,674 (Aug. 16, 2011 Kamen) “Device and Method for Food Management”; and U.S. patent applications 20080255955 (Oct. 16, 2008 Simons-Nikolova) “Weight Management System with Simple Data Input”, 20100205209 (Aug. 12, 2010 Jokinen) “Method and System for Monitoring a Personal Intake”, and 20130006807 (Jan. 3, 2013 Bai et al.) “Guideline-Based Food Purchase Management”.

### 13. Food Scale

Prior art in this category includes automated food scales with a computer interface that records the weight of a specific portion of food before it is consumed. Sometimes such food scales are stand-alone devices. Sometimes such food scales are incorporated into place settings (such as a specialized food-weighing plate, glass, or utensil). The vast majority of prior art in this category depends on some type of specific action by the person to record the type of food that is on the scale. Once the type of food is manually entered, converting it into estimates of specific nutrients or calories can then be done in a relatively straight-forward manner using a computerized database.

Prior art in this category has the same compliance problems that plague other manual food logging methods. Will a person really weigh each bit of food on which they snack throughout the day? Will they bring a food scale to social eating situations and use it there? Will a person consistently identify each type of food that they eat and enter this information into the scale device? These questions highlight some of the potential disadvantages of this category of art for monitoring food consumption.

Examples of prior art that appear to be best classified in this category include: U.S. Pat. No. 4,387,777 (Jun. 14, 1983 Ash) “Calorie Counting Method and Apparatus”, U.S. Pat. No. 4,875,533 (Oct. 24, 1989 Mihara et al.) “Automatic Weight Detecting Device”, U.S. Pat. No. 4,911,256 (Mar. 27, 1990 Attikiouzel) “Dietetic Measurement Apparatus”, U.S. Pat. No. 5,033,561 (Jul. 23, 1991 Hettinger) “Diet Control Device”, U.S. Pat. No. 5,233,520 (Aug. 3, 1993 Kretsch et al.) “Method and System for Measurement of Intake of Foods, Nutrients and Other Food Components in the Diet”, U.S. Pat. No. 5,388,043 (Feb. 7, 1995 Hettinger) “Diet and Behavioral Control Device”, U.S. Pat. No. 5,817,006 (Oct. 6, 1998 Bergh et al.) “Method and Apparatus for Measurement of Eating Speed”, and U.S. Pat. No. 6,425,862 (Jul. 30, 2002 Brown) “Interactive Furniture for Dieters”; and U.S. patent applications 20020124017 (Sep. 5, 2002 Mault) “Personal Digital Assistant with Food Scale Accessory”, 20060263750 (Nov. 23, 2006 Gordon) “Personal Nutrition Control Devices”, 20070028453 (Feb. 8, 2007 Crow) “Portion Control Serving Utensils”, 20070050058 (Mar. 1, 2007 Zuziak et al.) “Placemat for Calculating and Monitoring Calorie Intake”, 20070173703 (Jul. 26, 2007 Lee et al.) “Method, Apparatus, and Medium for Managing Weight by Using Calorie Consumption Information”, and 20120055718 (Mar. 8, 2012 Chen) “Electronic Scale for Recording Health Administration Data”.

### 14. Portion Size Control

Prior art in this category includes specific-size food containers, place settings, and/or serving utensils that standardize the portion sizes and/or bite sizes of food that a person consumes. Such prior art is heavily dependent on specific human actions (apart from the actual act of eating) to be successful. Food must be consistently stored, apportioned, served, and eaten using the specific containers, place settings, and/or serving utensils. Hand-held snacks consumed from a bag, for example, are not easily monitored by this approach. Also, a person can easily prepare food without using the specific containers. Further, such art by itself is not useful for food identification. Food identification requires further specific human action. For these reasons, this approach has significant limitations for consistent measurement and modification of food intake.

Examples of prior art that appear to be best classified in this category include: U.S. patents U.S. Pat. No. 4,075,769 (Feb. 28, 1978 Young) “Method and Article for Weight Reduction” and U.S. Pat. No. 7,044,739 (May 16, 2006 Matson) “System for Controlled Nutrition Consumption”; and U.S. patent applications 20050014111 (Jan. 20, 2005 Matson) “System for Controlled Nutrition Consumption”, 20100125181 (May 20, 2010 Hyde et al.) “Food Content Detector”, 20120031805 (Feb. 9, 2012 Stolarczyk) “Daily Meal Planning System”, 20120077154 (Mar. 29, 2012 Highet et al.) “Incrementally-Sized Standard-Sized Eating-Ware System for Weight Management”, and 20120144912 (Jun. 14, 2012 Kates et al.) “Portion Control System for Weight Loss and Maintenance”.

### 15. Mouth Size or Function Modification

This category of prior art includes devices and methods that limit mouth capacity or function so that a person eats less. In an example, a bulky device can be attached within a person's oral cavity in order to reduce the size of the cavity so that a person eats less food with each mouthful. This assumes that the person will not simply eat more mouthfuls to compensate. In another example, a device can be attached within the person's mouth to create resistance to chewing motion so that eating takes more work. The intent is that the person will eat less if eating requires more effort. In an example, a device can block consumption of solid food. This assumes that blocking solid food is an effective way to modify a person's diet to manage their weight. In an example, a device can physically cover or shield a person's tongue and taste buds so that they eat less. This assumes that such a device will be tolerated and will not be removed.

It is not clear from the prior art how such devices could be selectively used to allow consumption of healthy food, but discourage consumption of unhealthy food. Also, the prior art does not disclose how such devices would allow moderate consumption of certain foods but limit excess consumption of those foods. Also, if such a device is removable, then it requires consistent voluntary compliance by the person in order to be effective.

Examples of prior art that appear to be best classified in this category include: U.S. patents U.S. Pat. No. 3,818,906 (Jun. 25, 1974 Stubbs) “Apparatus for Controlling Eating and Smoking Habits”, U.S. Pat. No. 4,471,771 (Sep. 18, 1984 Brown) “Oral Weight Control Device”, U.S. Pat. No. 4,738,259 (Apr. 19, 1988 Brown et al.) “Dental Appliance for Weight Control”, U.S. Pat. No. 5,924,422 (Jul. 20, 1999 Gustafson) “Oral Device to Aid Weight Control”, U.S. Pat. No. 5,979,449 (Nov. 9, 1999 Steer) “Oral Appliance Device and Method for use Thereof for Appetite Suppression”, U.S. Pat. No. 6,422,243 (Jul. 23, 2002 Daram) “Taste Bud Shield and Method of Using Same”, and U.S. Pat. No. 8,230,865 (Jul. 31, 2012 Shalon) “Palatal Implant”; and U.S. patent applications 20030059737 (Mar. 27, 2003 Hall) “Obesity Treatment Aid”, 20050287495 (Dec. 29, 2005 Longley) “Dental Appliance for Weight Management”, and 20120109051 (May 3, 2012 Harrell) “Devices, Methods, and Kits for Taste Modification and Controlling Food Intake”.

### 16. Chewing and Swallowing Monitoring

Prior art in this category includes devices that monitor the chewing and/or swallowing actions that are associated with food consumption. In various examples, such devices can monitor chewing and/or swallowing by a method selected from the group consisting of: monitoring and analyzing sounds from a person's body to differentiate chewing and/or swallowing sounds from other sounds such as speaking; monitoring electromagnetic energy from a person's mouth muscles or internal gastrointestinal organs; and monitoring movement of a person's mouth or internal gastrointestinal organs.

Prior art in this category can be more automatic than art in many of the prior categories with respect to detecting when a person consumes food. Some art in this category can even generally differentiate between consumption of solid food vs. liquid food based on differences in sonic energy or electromagnetic energy. However, art in this category is generally very limited with respect to more-specific identification of what type of food a person is consuming. Also, a person can confuse or circumvent such a device by putting generally-solid food in a blender or by freezing generally-liquid food. Art in this category still relies on specific human actions to record food type apart from the actual action of eating. Also, since there can be different amounts of food per swallow, determination of food quantity based on the number of swallows can be problematic. Accordingly, prior art in this category has a number of limitations for measuring and modifying the types and quantities of food consumed.

Examples of prior art that appear to be best classified in this category include: U.S. patents U.S. Pat. No. 4,355,645 (Oct. 26, 1982 Mitani et al.) “Device for Displaying Masticatory Muscle Activities”, U.S. Pat. No. 5,067,488 (Nov. 26, 1991 Fukada et al.) “Mastication Detector and Measurement Apparatus and Method of Measuring Mastication”, U.S. Pat. No. 5,263,491 (Nov. 23, 1993 Thornton) “Ambulatory Metabolic Monitor”, U.S. Pat. No. 6,135,950 (Oct. 24, 2000 Adams) “E-fit Monitor”, U.S. Pat. No. 7,330,753 (Feb. 12, 2008 Policker et al.) “Analysis of Eating Habits”, U.S. Pat. No. 7,840,269 (Nov. 23, 2010 Policker et al.) “Analysis of Eating Habits”, U.S. Pat. No. 7,840,269 (Nov. 23, 2010 Policker et al.) “Analysis of Eating Habits”, and U.S. Pat. No. 7,914,468 (Mar. 29, 2011 Shalon et al.) “Systems and Methods for Monitoring and Modifying Behavior”; and U.S. patent applications 20040147816 (Jul. 29, 2004 Policker et al.) “Analysis of Eating Habits”, 20050283096 (Dec. 22, 2005 Chau et al.) “Apparatus and Method for Detecting Swallowing Activity”, 20060064037 (Mar. 23, 2006 Shalon et al.) “Systems and Methods for Monitoring and Modifying Behavior”, 20060064037 (Mar. 23, 2006 Shalon et al.) “Systems and Methods for Monitoring and Modifying Behavior”, 20060064037 (Mar. 23, 2006 Shalon et al.) “Systems and Methods for Monitoring and Modifying Behavior”, 20070299320 (Dec. 27, 2007 Policker et al.) “Analysis of Eating Habits”, 20070299320 (Dec. 27, 2007 Policker et al.) “Analysis of Eating Habits”, 20100076345 (Mar. 25, 2010 Soffer et al.) “Method, Device and System for Automatic Detection of Eating and Drinking”, 20110125063 (May 26, 2011 Shalon et al.) “Systems and Methods for Monitoring and Modifying Behavior”, 20110276312 (Nov. 10, 2011 Shalon et al.) “Device for Monitoring and Modifying Eating Behavior”, 20120101874 (Apr. 26, 2012 Ben-Haim et al.) “Charger With Data Transfer Capabilities”, and 20120203081 (Aug. 9, 2012 Leboeuf et al.) “Physiological and Environmental Monitoring Apparatus and Systems”. Another example of prior art that appears to be best classified in this category is WO 2002082968 (Policker) “Analysis of Eating Habits.”

17. Hand and/or Arm Motion Monitoring and Modification (Wrist)

This is the first of two categories of prior art wherein the intent is to detect and estimate food consumption by monitoring and analyzing hand and/or arm motion. This first category includes devices that are worn on a person's wrist or arm to directly monitor hand or arm motion. The second category (that follows this one) includes food utensils that indirectly monitor hand or arm motion when the utensil is held by a person and is used to bring food up to the person's mouth.

We have separated these devices into two categories because, even though they both monitor hand and arm motion, they have some different advantages and disadvantages. Devices worn on a person's wrist or arm have the advantage that they can be worn relatively continuously to monitor food consumption on a relatively continuous basis. Wrist-worn devices do not require that a person carry a specific motion-sensing food utensil everywhere that they go. However, a device that is worn on a person's wrist or arm can be subject to more false alarms (compared to a food utensil) due to non-food-consumption motions such as covering one's mouth when coughing, bringing a cigarette to one's mouth, or other hand-to-face gestures.

Many examples of devices in this category monitor hand and/or arm motion with an accelerometer. To the extent that there is a distinctive pattern of hand and/or arm movement associated with bringing food up to one's mouth, such a device can detect when food consumption is occurring. Such a device can also measure how rapidly or often the person brings their hand up to their mouth. A common use of such information is to encourage a person to eat at a slower pace. The idea that a person will eat less if they eat at a slower pace is based on the lag between food consumption and the feeling of satiety from internal gastric organs. If a person eats slower, then they will tend to not overeat past the point of internal identification of satiety. Detection of food consumption and approximate measurement of food consumption quantity that is based on hand or arm motion can also be useful for purposes other than slowing the pace of eating.

However, there are significant limitations to devices and methods in this category. First, such devices and methods do not provide good information concerning the types of food consumed. In this respect, they generally still rely on manual food identification methods. Second, although progress has been made to differentiate hand and/or arm motions that indicate food consumption from other types of hand and/or arm motions (such as covering one's mouth or brushing one's teeth), there remains imprecision with respect to quantification of food consumed based on analysis of hand-to-mouth movements. Third, it is tough to make such devices and methods tamper-resistant. A person can use non-conventional hand movements to eat, use a non-monitored hand to eat, eat larger bite sizes with each hand movement, remove the device from their wrist, or just ignore feedback from the device when they eat.

Examples of prior art that appear to be best classified in this category include: U.S. patents U.S. Pat. No. 3,885,576 (May 27, 1975 Symmes) “Wrist Band Including a Mercury Switch to Induce an Electric Shock”, U.S. Pat. No. 4,965,553 (Oct. 23, 1990 DelBiondo et al.) “Hand-Near-Mouth Warning Device”, U.S. Pat. No. 5,424,719 (Jun. 13, 1995 Ravid) “Consumption Control”, U.S. Pat. No. 5,563,850 (Oct. 8, 1996 Hanapole) “Food Intake Timer”, U.S. Pat. No. 8,112,281 (Feb. 7, 2012 Yeung et al.) “Accelerometer-Based Control of Wearable Audio Recorders”, and U.S. Pat. No. 8,310,368 (Nov. 13, 2012 Hoover et al.) “Weight Control Device”; and U.S. patent applications 20060197670 (Sep. 7, 2006 Breibart) “Method and Associated Device for Personal Weight Control”, 20080137486 (Jun. 12, 2008 Czarenk et al.) “Diet Watch”, and 20100194573 (Aug. 5, 2010 Hoover et al.) “Weight Control Device”.

18. Hand and/or Arm Motion Monitoring and Modification (Utensil)

Prior art in this category includes hand-held food serving utensils (such as forks or spoons) that indirectly monitor hand and/or arm motion to detect and estimate food consumption. Compared to the wrist-worn motion-detection devices that were discussed in the previous category, motion-detecting utensils can be less subject to false alarms because they are only used when the person consumes food. There are some recent examples of sophisticated food-analyzing utensils with sensors other than motion-sensors. Since they are qualitatively different than utensils with only motion sensors, we have put these more-sophisticated food-analyzing utensils in a separate category that follows in this categorization scheme.

Many examples of utensils in this category monitor motion with an accelerometer. Since the utensil is only used for food consumption, analysis of complex motion and differentiation of food consumption actions vs. other hand gestures is less important with a utensil than it is with a wrist-mounted device. Accordingly, some of the utensils in this category are quite simple. In the extreme, although crude, a single-axis accelerometer can be used. Other simple methods of measuring hand-to-mouth movement by a utensil are based on a simple holder or button on which the utensil is placed between mouthfuls. Another simple method is an internal fluid “horizontal level” or “lava lamp” feature attached to the utensil that is used to regulate the timing of hand-to-mouth motions.

The idea is that a person will eat less if they eat slower because there can be a lag between food consumption and identification of satiety by internal organs. If the person eats slower, then they will tend to not overeat past the point of internal identification of satiety. Detection of food consumption and approximate measurement of food consumption quantity based on hand or arm motion can also be useful for purposes other than slowing the pace of eating.

However, utensils with just a motion sensor do not provide good information concerning the type of food consumed. Also, compliance can be a huge issue for this approach. In order to be successful, a person has to bring the special utensil with them constantly and use it consistently whenever they eat. What happens when they are eating out in a social setting or eating a snack with their hands? For these reasons, special eating utensils with just a motion sensor are limited in their ability to consistently monitor and modify a person's food consumption.

Examples of prior art that appear to be best classified in this category include: U.S. patents U.S. Pat. No. 4,207,673 (Jun. 17, 1980 DiGirolamo et al.) “Cuttlery”, U.S. Pat. No. 4,914,819 (Apr. 10, 1990 Ash) “Eating Utensil for Indicating When Food May be Eaten Therewith and a Method for Using the Utensil”, U.S. Pat. No. 4,975,682 (Dec. 4, 1990 Kerr et al.) “Meal Minder Device”, U.S. Pat. No. 5,299,356 (Apr. 5, 1994 Maxwell) “Diet Eating Utensil”, U.S. Pat. No. 5,421,089 (Jun. 6, 1995 Dubus et al.) “Fork with Timer”, and U.S. Pat. No. 8,299,930 (Oct. 30, 2012 Schmid-Schonbein et al.) “Devices, Systems and Methods to Control Caloric Intake”; and U.S. patent applications 20070098856 (May 3, 2007 LePine) “Mealtime Eating Regulation Device”, 20080276461 (Nov. 13, 2008 Gold) “Eating Utensil Capable of Automatic Bite Counting”, 20090253105 (Oct. 8, 2009 Lepine) “Device for Regulating Eating by Measuring Potential”, 20100109876 (May 6, 2010 Schmid-Schonbein et al.) “Devices, Systems and Methods to Control Caloric Intake”, 20100240962 (Sep. 23, 2010 Contant) “Eating Utensil to Monitor and Regulate Dietary Intake”, and 20120115111 (May 10, 2012 Lepine) “Mealtime Eating Regulation Device”.

19. Utensil with Sensor Other than Motion Sensor

Prior art in this category includes food utensils with sensors other than motion sensors that are used to measure food consumption. Such art in this category is relatively innovative and there are relatively few examples to date. Prior art in this category represents an important step toward automated measurement of food consumption. In various examples, a utensil in this category can measure the volume, mass, density, or general composition of a bite-size portion of food that is transported by the utensil to a person's mouth.

However, a significant limitation of art in this category is that it relies on a person's compliance. The person must use the utensil each time that they eat anything in order for the system to successfully monitor food consumption. If a person eats food without using the utensil (e.g. when dining in a social setting or when eating a snack by hand), then the system is unaware of this food consumption. This can be problematic and the prior art does not offer a solution to this problem.

Examples of prior art that appear to be best classified in this category include: U.S. patents U.S. Pat. No. 8,229,676 (Jul. 24, 2012 Hyde et al.) “Food Content Detector”, U.S. Pat. No. 8,285,488 (Oct. 9, 2012 Hyde et al.) ibid., U.S. Pat. No. 8,290,712 (Oct. 16, 2012 Hyde et al.) ibid., U.S. Pat. No. 8,321,141 (Nov. 27, 2012 Hyde et al.) ibid., and U.S. Pat. No. 8,355,875 (Jan. 15, 2013 Hyde et al.) ibid.; and U.S. patent applications 20100125176 (May 20, 2010 Hyde et al.) ibid., 20100125177 (May 20, 2010 Hyde et al.) ibid., 20100125178 (May 20, 2010 Hyde et al.) ibid., 20100125179 (May 20, 2010 Hyde et al.) ibid., 20100125180 (May 20, 2010 Hyde et al.) ibid., 20100125181 (May 20, 2010 Hyde et al.) ibid., 20100125417 (May 20, 2010 Hyde et al.) ibid., 20100125418 (May 20, 2010 Hyde et al.) ibid., 20100125419 (May 20, 2010 Hyde et al.) ibid., 20100125420 (May 20, 2010 Hyde et al.) ibid., and 20110184247 (Jul. 28, 2011 Contant et al.) “Comprehensive Management of Human Health”.

### 20. Other Modification of Eating Speed

This category is a catch-all for other prior art that seeks to modify eating speed using methods that are not covered by prior categories. Examples of prior art in this category include “bite traffic light” devices and sound-activating timers that signal when a person can take another bite of food. Such devices differ from earlier devices because they are not incorporated into a utensil or a wrist-worn band.

Compliance issues are a major issue with this approach. Will a person consistently use and obey a “bite traffic light” in order to time the speed at which they take bites of food? Will a person consistently tap an application on a touch screen to time the speed at which they take bites of food? Such art might be helpful for some people with strong self-discipline, but these people might have enough self-discipline to achieve the same effect by just watching a clock or just eating slowly without any automated guidance. Better methods for measuring and monitoring food consumption are needed for people without such strong self-discipline.

Examples of prior art that appear to be best classified in this category include: U.S. Pat. No. 5,908,301 (Jun. 1, 1999 Lutz) “Method and Device for Modifying Behavior”, U.S. Pat. No. 6,473,368 (Oct. 29, 2002 Stanfield) “Consumption Controller”, and U.S. Pat. No. 6,765,488 (Jul. 20, 2004 Stanfield) “Enhanced Consumption Controller”; and patent application 20120021388 (Jan. 26, 2012 Arbuckle et al.) “System and Method for Weight Management”.

### 21. Photo Identification of Food (Bar Code or Other Packaging-Based Code)

Prior art in this category includes devices and methods for identifying food consumption based on photo identification of food using bar codes or other packaging-based codes. If consumed food has a bar code (or other packaging-based code) then it is relatively easy for a system to associate specific nutrients and/or total calories with that food.

However, there are several limitations to this approach. First, a person may eat food that is not identified by bar codes or other packaging-based codes. Food served in restaurants or in other people's homes is unlikely to be identified by such codes. Also, even in a grocery store, not all food is identified by such codes. Second, a person may not eat all of the food that is identified by such codes. Other people may eat some of the food in a given package. Also, some of the food in a given package may be thrown out. Also, depending on the longevity of the food, some food in a given package may be eaten soon after purchase and the rest may be eaten long afterwards. Accordingly, it can be problematic using such codes to make associations between food eaten by a specific person in a specific time period and the person's success in achieving weight management goals during that time period.

Examples of prior art that appear to be best classified in this category include: U.S. patents U.S. Pat. No. 5,819,735 (Oct. 13, 1998 Mansfield et al.) “Device and Method for Monitoring Dietary Intake of Calories And Nutrients” and U.S. Pat. No. 6,283,914 (Sep. 4, 2001 Mansfield et al.) “Device and Method for Monitoring Dietary Intake of Calories and Nutrients”; and U.S. patent applications 20030163354 (Aug. 28, 2003 Shamoun) “Device for Collecting and Analyzing Nutritional Data and Method Therefor”, 20030208110 (Nov. 6, 2003 Mault et al.) “Physiological Monitoring using Wrist-Mounted Device”, 20060189853 (Aug. 24, 2006 Brown) “Method and System for Improving Adherence with a Diet Program or Other Medical Regimen”, 20060229504 (Oct. 12, 2006 Johnson) “Methods and Systems for Lifestyle Management”, 20070059672 (Mar. 15, 2007 Shaw) “Nutrition Tracking Systems and Methods”, and 20090176526 (Jul. 9, 2009 Altman) “Longitudinal Personal Health Management System Using Mobile Data Capture”.

### 22. Photo Identification of Food (Manual Picture Taking and Identification)

Prior art in this category includes image-based devices and methods that require specific voluntary human action associated with each food consumption event (apart from the actual act of eating) in order: to take pictures of food during food consumption; and to identify the types and quantities of food consumed based on those pictures. In this category, neither picture taking nor food identification is automated. In an example, such art can include having a person aim a camera-equipped mobile electronic device toward food each time that the person eats and requiring that the person identify the type and quantity of food consumed based on the resulting pictures.

In an example, food identification by a person can occur in real-time (before, during, or immediately after a meal) using voice recognition or a menu-driven user interface. In another example, food identification by a person can occur later, long after the meal. In an example, food identification can be done by the person whose food consumption is being monitored and measured. In an example, food identification can be done by someone else.

Such image-based food logging systems are an improvement over recording food consumed with a pencil and paper. However, these devices and systems still require manual intervention to aim an imaging device toward a food source and to take at least one picture each time that the person eats something. Accordingly, they depend heavily on the person's compliance. These devices and methods can be time-consuming (having to aim the field of vision toward food), easy to circumvent (a person may simply not take pictures of some food consumed), and embarrassing to use social dining situations. This can lead to low long-term compliance.

Any approach that depends on voluntary human action each time that a person eats anything is difficult to make tamper-resistant. It is very easy for someone to “cheat” by simply not taking pictures of some consumed food items. Also, even if the person does consistently takes pictures of every meal or snack that they eat, then they may be tempted to postpone the manual task of food identification for hours or days after a meal has occurred. This can cause inaccuracy. How many chips were left in that bag in the picture? Is that a “before” or “after” picture of that gallon of ice cream? Delays in food identification can lead to imprecision in identification of the types and quantities of food consumed.

Examples of prior art that appear to be best classified in this category include U.S. patent applications: 20020047867 (Apr. 25, 2002 Mault et al.) “Image Based Diet Logging”, 20020109600 (Aug. 15, 2002 Mault et al.) “Body Supported Activity and Condition Monitor”, 20070030339 (Feb. 8, 2007 Findlay et al.) “Method, System and Software for Monitoring Compliance”, 20090112800 (Apr. 30, 2009 Athsani) “System and Method for Visual Contextual Search”, and 20090219159 (Sep. 3, 2009 Morgenstern) “Method and System for an Electronic Personal Trainer”.

### 23. Photo Identification of Food (Manual Picture Taking and Automatic Identification)

Prior art in this category includes image-based devices and methods that require specific voluntary human actions associated with each food consumption event (apart from the actual act of eating) in order to take pictures of food during consumption. However, these devices and methods automatically identify the types and quantities of food consumed based on these pictures. In various examples, automatic identification of food types and quantities can be based on: color and texture analysis; image segmentation; image pattern recognition; volumetric analysis based on a fiduciary market or other object of known size; and/or three-dimensional modeling based on pictures from multiple perspectives. In an example, food identification can occur before or during a meal. In an example, a mobile phone application can transmit images to a remote location where automatic food identification occurs.

In some examples, food identification is an interactive process that combines automatic and manual methods of food identification. In this category, picture taking is not automated. In an example, such art can include having a person aim a camera-equipped mobile electronic device toward food to take pictures every time that the person eats food.

Such image-based consumption monitoring systems are useful, but still require specific actions by the person to aim an imaging device toward a food source and to take at least one picture of food each time that the person eats something. Accordingly, such art depends on the person's compliance. Such devices and methods can be time-consuming, easy to circumvent, and embarrassing in social dining situations. Any approach that depends on voluntary human action each time that a person eats anything is difficult to make tamper-resistant. It is very easy for someone to eat something without first taking a picture of it.

Examples of prior art that appear to be best classified in this category include: U.S. patents U.S. Pat. No. 6,513,532 (Feb. 4, 2003 Mault et al.) “Diet and Activity Monitoring Device”, U.S. Pat. No. 8,345,930 (Jan. 1, 2013 Tamrakar et al.) “Method for Computing Food Volume in a Method for Analyzing Food”, and U.S. Pat. No. 8,363,913 (Jan. 29, 2013 Boushey et al.) “Dietary Assessment System and Method”; and U.S. patent applications 20010049470 (Dec. 6, 2001 Mault et al.) “Diet and Activity Monitoring Device”, 20020027164 (Mar. 7, 2002 Mault et al.) “Portable Computing Apparatus Particularly Useful in a Weight Management Program”, 20030065257 (Apr. 3, 2003 Mault et al.) “Diet and Activity Monitoring Device”, 20030076983 (Apr. 24, 2003 Cox) “Personal Food Analyzer”, 20080267444 (Oct. 30, 2008 Simons-Nikolova) “Modifying a Person's Eating and Activity Habits”, 20100111383 (May 6, 2010 Boushey et al.) “Dietary Assessment System and Method”, 20100173269 (Jul. 8, 2010 Puri et al.) “Food Recognition Using Visual Analysis and Speech Recognition”, 20100191155 (Jul. 29, 2010 Kim et al.) “Apparatus for Calculating Calories Balance by Classifying User's Activity”, 20100332571 (Dec. 30, 2010 Healey et al.) “Device Augmented Food Identification”, 20110182477 (Jul. 28, 2011 Tamrakar et al.) “Method for Computing Food Volume in a Method for Analyzing Food”, 20110318717 (Dec. 29, 2011 Adamowicz) “Personalized Food Identification and Nutrition Guidance System”, 20120170801 (Jul. 5, 2012 De Oliveira et al.) “System for Food Recognition Method Using Portable Devices Having Digital Cameras”, 20120179665 (Jul. 12, 2012 Baarman et al.) “Health Monitoring System”, 20120313776 (Dec. 13, 2012 Utter) “General Health and Wellness Management Method and Apparatus for a Wellness Application Using Data from a Data-Capable Band”, 20120326873 (Dec. 27, 2012 Utter) “Activity Attainment Method and Apparatus for a Wellness Application Using Data from a Data-Capable Band”, and 20130004923 (Jan. 3, 2013 Utter) “Nutrition Management Method and Apparatus for a Wellness Application Using Data from a Data-Capable Band”.

### 24. Photo Identification of Food (Automatic Picture Taking and Identification)

Prior art in this category includes image-based devices and methods that automatically take and analyze pictures of food in order to identify the types and quantities of food consumed without the need for specific human action associated with each food consumption event (apart from the actual act of eating). In an example, automatic picture taking can occur using a camera that the person wears continually. In an example, a wearable camera can take pictures continually. In various examples, automatic identification of food types and quantities can be based on: color and texture analysis; image segmentation; image pattern recognition; volumetric analysis based on a fiduciary market or other object of known size; and/or three-dimensional modeling based on pictures from multiple perspectives. As an advantage over freestanding mobile imaging devices, wearable imaging devices offer a higher degree of automation.

Although art in this category is an innovative advance in the field, it still has at least three significant limitations that have not been fully addressed by the prior art. First, there is a trade-off between the measurement advantages of a continually-imaging wearable camera and the potential intrusion into a person's privacy. How can one achieve the measurement advantages of the wearable-imaging approach to food consumption monitoring with minimal intrusion into a person's privacy? Second, how does one address the possibility that a person can tamper with, or circumvent, such a device? Prior art in this category does not offer a tamper-resistant device.

Third, there are limitations to how accurately an image-based system can identify the composition of food. For example, many types of food, especially liquids, look similar. For example, if a beverage is not consumed in its original container, how can an image-based system know whether the beverage is high sugar vs. low sugar, or unhealthy vs. healthy? What is that sandwiched between two buns in a burger? Is it beef or turkey or a “veggie burger”? For these reasons, even though image-based prior art in this category is innovative and useful, there remains a need for better methods for automatically measuring the types and quantities of food consumption.

Examples of prior art that appear to be best classified in this category include U.S. Pat. No. 6,508,762 (Jan. 21, 2003 Karnieli) “Method for Monitoring Food Intake” and patent applications 20020022774 (Feb. 21, 2002 Karnieli) “Method for Monitoring Food Intake”, and 20090012433 (Jan. 8, 2009 Fernstrom et al.) “Method, Apparatus and System for Food Intake and Physical Activity Assessment”.

### 25. Gastric Band

With this category, we now move from devices and methods that are primarily used externally to the human body to devices and methods that are primarily implanted within the human body. Prior art in this particular category includes implantable devices that externally constrain the cross-sectional size of a member of a person's gastrointestinal tract (such as their stomach) to constrain the volume or amount of food that a person consumes. In an example, art in this category includes gastric bands that externally encircle and constrain expansion of the upper portion of a person's stomach in order to limit the volume or amount of food that passes into the person's stomach. Many of the devices in this category are adjustable in size, allowing post-operative adjustment of the external circumference of the portion of the gastrointestinal organ which the device encircles. We have separated out such devices which include sensors (and can self-adjust) in a category following this one.

Although devices in this category are innovative and have benefited many people, such devices still have limitations. First, such devices in the prior art are relatively food blind. They blindly reduce intake of all types of food. The prior art does not specify how they could be used to selectively reduce intake of unhealthy food while allowing normal consumption of healthy food. Second, such devices can irritate or harm the tissue of the gastrointestinal organ which they encircle. Third, although such devices can limit the size and flow of food entering a person's stomach, such devices do not limit the overall quantity of food that enters a person's stomach over time. For example, if a person wishes to melt an entire gallon of ice cream and then ingest it, a gastric band will not prevent this. There remains a need for better approaches for selectively modifying a person's food consumption.

Examples of prior art that appear to be best classified in this category include: U.S. patents U.S. Pat. No. 6,547,801 (Apr. 15, 2003 Dargent et al.) “Gastric Constriction Device”, U.S. Pat. No. 6,551,235 (Apr. 22, 2003 Forsell) “Implantable Pump”, U.S. Pat. No. 6,966,875 (Nov. 22, 2005 Longobardi) “Adjustable Gastric Implant”, U.S. Pat. No. 7,775,967 (Aug. 17, 2010 Gertner) “Closed Loop Gastric Restriction Devices and Methods”, U.S. Pat. No. 7,798,954 (Sep. 21, 2010 Birk et al.) “Hydraulic Gastric Band with Collapsible Reservoir”, U.S. Pat. No. 7,909,754 (Mar. 22, 2011 Hassler et al.) “Non-Invasive Measurement of Fluid Pressure in an Adjustable Gastric Band”, U.S. Pat. No. 7,972,346 (Jul. 5, 2011 Bachmann et al.) “Telemetrically Controlled Band for Regulating Functioning of a Body Organ or Duct, and Methods of Making, Implantation And Use”, U.S. Pat. No. 8,034,065 (Oct. 11, 2011 Coe et al.) “Controlling Pressure in Adjustable Restriction Devices”, U.S. Pat. No. 8,043,206 (Oct. 25, 2011 Birk) “Self-Regulating Gastric Band with Pressure Data Processing”, U.S. Pat. No. 8,100,870 (Jan. 24, 2012 Marcotte et al.) “Adjustable Height Gastric Restriction Devices and Methods”, U.S. Pat. No. 8,137,261 (Mar. 20, 2012 Kierath et al.) “Device for the Treatment of Obesity”, U.S. Pat. No. 8,292,800 (Oct. 23, 2012 Stone et al.) “Implantable Pump System”, U.S. Pat. No. 8,317,677 (Nov. 27, 2012 Bertolote et al.) “Mechanical Gastric Band with Cushions”, and U.S. Pat. No. 8,323,180 (Dec. 4, 2012 Birk et al.) “Hydraulic Gastric Band with Collapsible Reservoir”; and U.S. patent applications 20070156013 (Jul. 5, 2007 Birk) “Self-Regulating Gastric Band with Pressure Data Processing”, 20070265645 (Nov. 15, 2007 Birk et al.) “Hydraulic Gastric Band Collapsible Reservoir”, 20070265646 (Nov. 15, 2007 Mccoy et al.) “Dynamically Adjustable Gastric Implants”, and 20080275484 (Nov. 6, 2008 Gertner) “Per Os Placement of Extragastric Devices”.

Examples of prior art that appear to be best classified in this category also include U.S. patent applications: 20090157106 (Jun. 18, 2009 Marcotte et al.) “Adjustable Height Gastric Restriction Devices and Methods”, 20090171375 (Jul. 2, 2009 Coe et al.) “Controlling Pressure in Adjustable Restriction Devices”, 20090204131 (Aug. 13, 2009 Ortiz et al.) “Automatically Adjusting Band System with MEMS Pump”, 20090204132 (Aug. 13, 2009 Ortiz et al.) “Automatically Adjusting Band System”, 20090216255 (Aug. 27, 2009 Coe et al.) “Controlling Pressure in Adjustable Restriction Devices”, 20090270904 (Oct. 29, 2009 Birk et al.) “Remotely Adjustable Gastric Banding System”, 20090312785 (Dec. 17, 2009 Stone et al.) “Implantable Pump System”, 20100228080 (Sep. 9, 2010 Tavori et al.) “Apparatus and Methods for Corrective Guidance of Eating Behavior after Weight Loss Surgery”, 20100234682 (Sep. 16, 2010 Gertner) “Closed Loop Gastric Restriction Devices and Methods”, 20100324358 (Dec. 23, 2010 Birk et al.) “Hydraulic Gastric Band with Collapsible Reservoir”, 20110130626 (Jun. 2, 2011 Hassler et al.) “Non-Invasive Measurement of Fluid Pressure in an Adjustable Gastric Band”, 20110184229 (Jul. 28, 2011 Raven et al.) “Laparoscopic Gastric Band with Active Agents”, 20110201874 (Aug. 18, 2011 Birk et al.) “Remotely Adjustable Gastric Banding System”, 20110207994 (Aug. 25, 2011 Burrell et al.) “Methods and Devices for Treating Morbid Obesity Using Hydrogel”, 20110207995 (Aug. 25, 2011 Snow et al.) “Inductively Powered Remotely Adjustable Gastric Banding System”, 20110208216 (Aug. 25, 2011 Fobi et al.) “Gastric Bypass Band and Surgical Method”, and 20110270025 (Nov. 3, 2011 Fridez et al.) “Remotely Powered Remotely Adjustable Gastric Band System”.

Examples of prior art that appear to be best classified in this category also include U.S. patent applications: 20110270030 (Nov. 3, 2011 Birk et al.) “Hydraulic Gastric Band with Collapsible Reservoir”, 20110275887 (Nov. 10, 2011 Birk) “Self-Regulating Gastric Band with Pressure Data Processing”, 20110306824 (Dec. 15, 2011 Perron et al.) “Remotely Adjustable Gastric Banding System”, 20110313240 (Dec. 22, 2011 Phillips et al.) “Flow Restrictor and Method for Automatically Controlling Pressure for a Gastric Band”, 20120046674 (Feb. 23, 2012 Augarten et al.) “Power Regulated Implant”, 20120059216 (Mar. 8, 2012 Perron) “Remotely Adjustable Gastric Banding System”, 20120067937 (Mar. 22, 2012 Menzel) “Internal Gastric Bander for Obesity”, 20120083650 (Apr. 5, 2012 Raven) “Systems and Methods for Adjusting Gastric Band Pressure”, 20120088962 (Apr. 12, 2012 Franklin et al.) “Self-Adjusting Gastric Band”, 20120095288 (Apr. 19, 2012 Snow et al.) “Self-Adjusting Gastric Band”, 20120130273 (May 24, 2012 Hassler et al.) “Non-Invasive Measurement of Fluid Pressure in an Adjustable Gastric Band”, 20120190919 (Jul. 26, 2012 Phillips et al.) “Assembly and Method for Automatically Controlling Pressure for a Gastric Band”, 20120197069 (Aug. 2, 2012 Lau et al.) “Assembly and Method for Automatically Controlling Pressure for a Gastric Band”, 20120215061 (Aug. 23, 2012 Fridez et al.) “Hydraulic Gastric Band with Reversible Self-Opening Mechanism”, 20120215062 (Aug. 23, 2012 Coe) “Remotely Adjustable Gastric Banding Device”, 20120296157 (Nov. 22, 2012 Tozzi et al.) “Medical Device Comprising an Artificial Contractile Structure”, and 20120302936 (Nov. 29, 2012 Belhe et al.) “External Anchoring Configurations for Modular Gastrointestinal Prostheses”.

26. Gastric Band with Sensor

Prior art in this category is similar to that of the previous category except for the addition of a sensor and the possibility of self-adjusting operation. The vast majority of sensors in this category are pressure sensors. The addition of a pressure sensor to a gastric band enables remote or automatic adjustment of the size of the constraining band in response to pressure from the external circumference of the encircled gastrointestinal organ. This can help to reduce irritation or harm of organ tissue by a constraining band, can enable post-operative refinement of therapy, and can help to reduce undesirable regurgitation. However, the other limitations that were identified with respect to gastric bands in the above category are still generally applicable to gastric bands in this category.

Examples of prior art that appear to be best classified in this category include: U.S. patents U.S. Pat. No. 7,775,966 (Aug. 17, 2010 Dlugos et al.) “Non-Invasive Pressure Measurement in a Fluid Adjustable Restrictive Device”, U.S. Pat. No. 7,879,068 (Feb. 1, 2011 Dlugos et al.) “Feedback Sensing for a Mechanical Restrictive Device”, U.S. Pat. No. 8,251,888 (Aug. 28, 2012 Roslin et al.) “Artificial Gastric Valve”, and U.S. Pat. No. 8,308,630 (Nov. 13, 2012 Birk et al.) “Hydraulic Gastric Band with Collapsible Reservoir”; and U.S. patent applications 20060173238 (Aug. 3, 2006 Starkebaum) “Dynamically Controlled Gastric Occlusion Device”, 20060199997 (Sep. 7, 2006 Hassler et al.) “Monitoring of a Food Intake Restriction Device”, 20060235448 (Oct. 19, 2006 Roslin et al.) “Artificial Gastric Valve”, 20080172072 (Jul. 17, 2008 Pool et al.) “Internal Sensors for Use with Gastric Restriction Devices”, 20090192534 (Jul. 30, 2009 Ortiz et al.) “Sensor Trigger”, 20100152532 (Jun. 17, 2010 Marcotte) “Gastric Band System with Esophageal Sensor”, 20100274274 (Oct. 28, 2010 Roslin et al.) “Artificial Gastric Valve”, 20110034760 (Feb. 10, 2011 Brynelsen et al.) “Feedback Systems and Methods to Enhance Obstructive and Other Obesity Treatments”, 20110245598 (Oct. 6, 2011 Gertner) “Closed Loop Gastric Restriction Devices and Methods”, and 20120108921 (May 3, 2012 Raven et al.) “Gastric Banding System Adjustment Based on a Satiety Agent Concentration Level”.

### 27. Gastrointestinal (GI) Bypass and Tissue Plication

A gastrointestinal bypass is the creation of a new route for food to travel through a person's gastrointestinal tract that is shorter and involves less absorption of nutrients than the normal route which food travels. In some examples, the creation of a gastrointestinal bypass is primarily a surgical procedure involving reconfiguration of gastrointestinal tissue that is not primarily dependent on an implantable medical device. In other examples, the creation of a gastrointestinal bypass depends on implantation of a specific medical device. In this category, we focus primarily the role of implantable medical devices in creating a gastric bypass.

Tissue plication involves the folding and/or compartmentalization of gastrointestinal tissue in order to change the flow and/or absorption of food in a person's gastrointestinal tract. In an example, stomach walls can be folded or compartmentalized by suturing or stapling tissue to reduce the surface area of the stomach to which food is exposed. Although one could argue that GI bypass and tissue plication should be in separate categories, we have grouped them together because they both involve altering natural tissue to change the pathway and absorption of food traveling through a person's gastrointestinal tract.

Gastrointestinal (GI) bypass and tissue plication can be very effective in reducing a person's food consumption and/or absorption of nutrients from food that is consumed. However, these approaches have some significant limitations. First, some of these operations are relatively invasive, including the health risks associated with the surgery and relatively-long recovery times. Second, most of these operations are non-reversible, even if they are unsuccessful or have adverse side effects. Third, prior art in this category blindly reduces absorption of nutrients from both healthy and unhealthy food. This can result in deficiencies of key nutrients. This is particularly problematic for procedures that are non-reversible.

Examples of prior art that appear to be best classified in this category include: U.S. Pat. No. 6,558,400 (May 6, 2003 Deem et al.) “Obesity Treatment Tools and Methods”, U.S. Pat. No. 6,572,629 (Jun. 3, 2003 Kalloo et al.) “Gastric Reduction Endoscopy”, U.S. Pat. No. 7,037,343 (May 2, 2006 Imran) “Stomach Prosthesis”, U.S. Pat. No. 7,037,344 (May 2, 2006 Kagan et al.) “Apparatus and Methods for Treatment of Morbid Obesity”, U.S. Pat. No. 7,141,071 (Nov. 28, 2006 Imran) “Implantable Digestive Tract Organ”, U.S. Pat. No. 7,288,099 (Oct. 30, 2007 Deem et al.) “Obesity Treatment Tools and Methods”, U.S. Pat. No. 7,288,101 (Oct. 30, 2007 Deem et al.) “Obesity Treatment Tools and Methods”, U.S. Pat. No. 7,503,922 (Mar. 17, 2009 Deem et al.) “Obesity Treatment Tools and Methods”, U.S. Pat. No. 7,510,559 (Mar. 31, 2009 Deem et al.) “Obesity Treatment Tools and Methods”, U.S. Pat. No. 7,601,178 (Oct. 13, 2009 Imran) “Stomach Peristalsis Device and Method”, U.S. Pat. No. 7,803,195 (Sep. 28, 2010 Levy et al.) “Obesity Treatment and Device”, U.S. Pat. No. 7,862,574 (Jan. 4, 2011 Deem et al.) “Obesity Treatment Tools and Methods”, U.S. Pat. No. 7,909,838 (Mar. 22, 2011 Deem et al.) “Obesity Treatment Tools and Methods”, U.S. Pat. No. 7,909,839 (Mar. 22, 2011 Fields) “Gastric Bypass Band and Surgical Method”, U.S. Pat. No. 7,931,694 (Apr. 26, 2011 Imran) “Stomach Peristalsis Device and Method”, U.S. Pat. No. 7,938,769 (May 10, 2011 Gertner) “Compressive Device for Percutaneous Treatment of Obesity”, U.S. Pat. No. 7,988,617 (Aug. 2, 2011 Gertner) “Extragastric Minimally Invasive Methods and Devices to Treat Obesity”, and U.S. Pat. No. 8,034,118 (Oct. 11, 2011 Imran) “Implantable Digestive Tract Organ”.

Examples of prior art that appear to be best classified in this category also include: U.S. Pat. No. 8,070,673 (Dec. 6, 2011 Gertner et al.) “Devices and Methods to Treat A Patient”, U.S. Pat. No. 8,075,577 (Dec. 13, 2011 Deem et al.) “Obesity Treatment Tools and Methods”, U.S. Pat. No. 8,080,022 (Dec. 20, 2011 Deem et al.) “Obesity Treatment Tools and Methods”, U.S. Pat. No. 8,080,025 (Dec. 20, 2011 Deem et al.) “Obesity Treatment Tools and Methods”, U.S. Pat. No. 8,123,765 (Feb. 28, 2012 Deem et al.) “Obesity Treatment Tools and Methods”, U.S. Pat. No. 8,137,366 (Mar. 20, 2012 Deem et al.) “Obesity Treatment Tools and Methods”, U.S. Pat. No. 8,137,367 (Mar. 20, 2012 Deem et al.) “Obesity Treatment Tools and Methods”, U.S. Pat. No. 8,147,441 (Apr. 3, 2012 Gannoe et al.) “Method and Device for Use in Endoscopic Organ Procedures”, U.S. Pat. No. 8,187,289 (May 29, 2012 Tacchino et al.) “Device and Method for the Therapy of Obesity”, U.S. Pat. No. 8,197,498 (Jun. 12, 2012 Coleman et al.) “Gastric Bypass Devices and Procedures”, U.S. Pat. No. 8,206,456 (Jun. 26, 2012 Stack et al.) “Restrictive and/or Obstructive Implant System for Inducing Weight Loss”, U.S. Pat. No. 8,211,128 (Jul. 3, 2012 Facundus et al.) “Multifunction Gastric Bypass Apparatus and Method”, U.S. Pat. No. 8,252,009 (Aug. 28, 2012 Weller et al.) “Devices and Methods for Placement of Partitions within a Hollow Body Organ”, and U.S. Pat. No. 8,287,554 (Oct. 16, 2012 Cerier et al.) “Method and Devices for Tissue Reconfiguration”.

Examples of prior art that appear to be best classified in this category also include U.S. patent applications: 20040122452 (Jun. 24, 2004 Deem et al.) “Obesity Treatment Tools and Methods”, 20040122453 (Jun. 24, 2004 Deem et al.) “Obesity Treatment Tools and Methods”, 20070093910 (Apr. 26, 2007 Imran) “Implantable Digestive Tract Organ”, 20070250083 (Oct. 25, 2007 Deem et al.) “Obesity Treatment Tools and Methods”, 20100004755 (Jan. 7, 2010 Imran) “Stomach Peristalsis Device and Method”, 20100145378 (Jun. 10, 2010 Gertner) “Percutaneous Gastroplasty”, 20100204723 (Aug. 12, 2010 Gertner) “Obesity Systems Placed Between the Abdominal Wall and Stomach”, 20110009887 (Jan. 13, 2011 Harris et al.) “Methods for Reducing Gastric Volume”, 20110009980 (Jan. 13, 2011 Levy et al.) “Obesity Treatment and Device”, 20110098725 (Apr. 28, 2011 Cox et al.) “Devices and Methods for Endolumenal Weight Loss Treatments”, 20110152899 (Jun. 23, 2011 Deem et al.) “Obesity Treatment Tools and Methods”, 20110152899 (Jun. 23, 2011 Deem et al.) “Obesity Treatment Tools and Methods”, 20110196504 (Aug. 11, 2011 Imran) “Stomach Peristalsis Device and Method”, and 20110208209 (Aug. 25, 2011 Ewers et al.) “Devices and Methods for Laparoscopic Gastric Tissue Reconfiguration”.

Examples of prior art that appear to be best classified in this category also include U.S. patent applications: 20110213385 (Sep. 1, 2011 Ewers et al.) “Delivery Systems and Methods for Gastric Reduction”, 20110295055 (Dec. 1, 2011 Albrecht et al.) “Methods and Devices for the Rerouting of Chyme to Induct Intestinal Brake”, 20120010459 (Jan. 12, 2012 Lau et al.) “Assembly and Method for Automatically Controlling Pressure for a Gastric Band”, 20120016392 (Jan. 19, 2012 Silverman et al.) “Method for Treating Morbid Obesity”, 20120022319 (Jan. 26, 2012 Muller) “Systems and Methods for Reducing Gastric Volume”, 20120071900 (Mar. 22, 2012 Vahid et al.) “Methods for Reduction of Gastric Lumen”, 20120101594 (Apr. 26, 2012 Fogel) “Endoscopic Implantable Device and Method for the Apposition of the Stomach Walls for Reducing the Stomach Internal Volume in a Weight Loss Surgery . . . ”, 20120116536 (May 10, 2012 Imran) “Implantable Digestive Tract Organ”, 20120160893 (Jun. 28, 2012 Harris et al.) “Methods and Devices for Reducing Gastric Volume”, 20120165843 (Jun. 28, 2012 Gannoe et al.) “Method and Device for use in Endoscopic Organ Procedures”, 20120165845 (Jun. 28, 2012 Harris et al.) “Methods and Devices for Reducing Gastric Volume”, 20120209400 (Aug. 16, 2012 Schurr) “Medical Implant”, 20120209400 (Aug. 16, 2012 Schurr) “Medical Implant”, 20120265224 (Oct. 18, 2012 Coleman et al.) “Gastric Bypass Devices and Procedures”, 20120296348 (Nov. 22, 2012 Saadat et al.) “Apparatus for Manipulating and Securing Tissue”, and 20120296354 (Nov. 22, 2012 Hsu et al.) “Methods and Devices for Treating Obesity and GERD by Intussuscepting a Portion of Stomach Tissue”.

### 28. Pumping Food Out of the Stomach Through an Intra-Abdominal Pathway

This novel and unusual category of prior art comprises an implantable intra-abdominal pathway and an accompanying pumping mechanism that allows a person to pump food out of their stomach. Using such a device, even if a person is unable to control what food they eat, the person can still avoid having the body absorb nutrients from the consumed food. This is a novel approach to the problem of excessive caloric intake, but there remain many unknowns with respect to its use. How will people view discharging partially-digested food through a permanent implantable intra-abdominal pathway as a method for losing weight? Will the connections between the intra-abdominal pathway, the person's actively-moving stomach, and the person's skin surface remain durable, secure, and sanitary?

Examples of prior art that appear to be best classified in this category include: U.S. patents U.S. Pat. No. 7,648,479 (Jan. 19, 2010 Solovay et al.) “Systems and Methods for Removing Ingested Material from a Stomach”, U.S. Pat. No. 7,740,624 (Jun. 22, 2010 Klein et al.) “Method for Treating Obesity by Extracting Food”, U.S. Pat. No. 7,815,629 (Oct. 19, 2010 Klein et al.) “Apparatus for Treating Obesity by Extracting Food”, U.S. Pat. No. 8,002,758 (Aug. 23, 2011 Kamen et al.) “Systems and Methods for Removing Ingested Material from a Stomach”, and U.S. Pat. No. 8,062,285 (Nov. 22, 2011 Langloss et al.) “Systems and Methods for Removing Ingested Material from a Stomach”; and U.S. patent applications U.S. Pat. No. 8,282,623 (Oct. 9, 2012 Klein et al.) “Method for Treating Obesity by Extracting Food”, 20050277900 (Dec. 15, 2005 Klein et al.) “Apparatus for Treating Obesity by Extracting Food”, 20080033345 (Feb. 7, 2008 Langloss et al.) “Systems and Methods for Removing Ingested Material from a Stomach”, 20080033364 (Feb. 7, 2008 Kamen et al.) “Systems and Methods for Removing Ingested Material from a Stomach”, 20080033365 (Feb. 7, 2008 Solovay et al.) “Systems and Methods for Removing Ingested Material from a Stomach”, 20080039809 (Feb. 14, 2008 Kamen et al.) “Systems and Methods for Removing Ingested Material from a Stomach”, 20080091146 (Apr. 17, 2008 Solovay et al.) “Shunt Apparatus for Treating Obesity by Extracting Food”, 20100106130 (Apr. 29, 2010 Solovay et al.) “Method for Treating Obesity by Extracting Food”, 20100106131 (Apr. 29, 2010 Klein et al.) “Method for Treating Obesity by Extracting Food”, 20100241090 (Sep. 23, 2010 Klein et al.) “Apparatus for Treating Obesity by Extracting Food”, 20110178480 (Jul. 21, 2011 Solovay et al.) “Shunt Apparatus for Treating Obesity by Extracting Food”, and 20110190719 (Aug. 4, 2011 Kamen et al.) “Systems And Methods for Removing Ingested Material from a Stomach”.

### 29. Gastric Tube

Prior art in this category includes insertion of a tube down into a person's gastrointestinal tract. Devices in this category, including gastric tubes, are generally used for feeding purposes rather than modification of food consumption or absorption. Nonetheless, we have included them here in this categorization scheme because tubes inserted into the gastrointestinal tract can be relevant to some approaches to modification of food consumption.

Examples of prior art that appear to be best classified in this category include: U.S. patents U.S. Pat. No. 7,794,425 (Sep. 14, 2010 Gobel) “Gastro-Esophageal Reflux Control System and Pump” and U.S. Pat. No. 7,967,780 (Jun. 28, 2011 Goebel) “Gastro-Esophageal Reflux Control System and Pump”; and U.S. patent applications 20080154191 (Jun. 26, 2008 Gobel) “Gastro-Esophageal Reflux Control System and Pump”, 20090062725 (Mar. 5, 2009 Goebel) “Gastro-Esophageal Reflux Control System and Pump”, 20100204669 (Aug. 12, 2010 Knight) “Enteral Feeding Safety Reservoir and System”, 20100217194 (Aug. 26, 2010 Pang) “Device for Tube Feeding”, 20100298812 (Nov. 25, 2010 Wolkenstorfer) “Catheter System”, and 20110082442 (Apr. 7, 2011 Solovay et al.) “Externally Reinforced Percutaneous Gastrostomy Tube with Customizable Smooth Tube Length”.

### 30. Enzyme Flow Modification

Prior art in this category includes diversion of enzymes that play a role in the digestion and absorption of food in the gastrointestinal tract. In various examples, the flow of enzymes into the gastrointestinal tract can be increased, decreased, or diverted. For example, enzymes can be diverted so that they are discharged into the gastrointestinal tract at a lower location, thereby reducing the digestion and absorption of food that passes through the gastrointestinal tract.

Examples of prior art that appear to be best classified in this category include: U.S. Pat. No. 7,833,279 (Nov. 16, 2010 Knudson et al.) “Pancreatic Exocrine Secretion Diversion Apparatus and Method”; and U.S. patent applications 20060106332 (May 18, 2006 Knudson et al.) “Pancreatic Exocrine Secretion Diversion Apparatus and Method”, 20110021968 (Jan. 27, 2011 Knudson et al.) “Pancreatic Exocrine Secretion Diversion Apparatus and Method”, 20120116285 (May 10, 2012 Duggirala) “Devices for Treating Obesity and Methods of Using Those Devices”, and 20120172782 (Jul. 5, 2012 Thompson) “Methods for Biliary Diversion”.

### 31. Gastrointestinal (GI) Volume or Pressure or Flow Modification

This relatively-broad category of prior art includes various devices that modify the interior volume of a gastrointestinal organ (such as the stomach), interior wall pressure of a gastrointestinal organ (such as the stomach), and/or food flow through a valve in a gastro-intestinal organ (such as the pyloric valve in the stomach). In various examples, art in this category can: occupy some of the interior volume of a gastrointestinal organ (such as an expandable gastric balloon in the stomach); apply pressure to the interior walls of a gastrointestinal organ (such as an expandable stomach stent); or mechanically modify the operation of a gastrointestinal valve (such as the operation of the pyloric valve within the stomach).

In an example, reducing the available space for food to occupy within the stomach can reduce the amount of food consumed and/or cause an earlier sensation of fullness. In an example, applying pressure to the interior walls of the stomach can cause an earlier sensation of fullness and reduce the amount of food consumed. In an example, reducing the outflow of food from the stomach by modifying the operation of the pyloric valve can lead to an earlier sensation of fullness and reduce food consumed.

However, there can be limitations to such devices. For example, the stomach can stretch even further when a balloon is implanted inside it or a stent is expanded within it, thwarting efforts to cause an earlier sensation of fullness or reduce food consumption. Also, even if a temporary balloon or stent is effective while implanted, that effect can be lost (or reversed) when the temporary balloon or stent is removed. In a worst case scenario, such a device can make the person worse off After removal of a balloon or stent, a stretched stomach can accommodate even more food than normal, causing the person to eat more than ever in the long run.

Examples of prior art that appear to be best classified in this category include: U.S. Pat. No. 4,133,315 (Jan. 9, 1979 Berman et al.) “Method and Apparatus for Reducing Obesity”, U.S. Pat. No. 4,416,267 (Nov. 22, 1983 Garren et al.) “Method and Apparatus for Treating Obesity”, U.S. Pat. No. 4,592,339 (Jun. 3, 1986 Kuzmak et al.) “Gastric Banding Device”, U.S. Pat. No. 4,694,827 (Sep. 22, 1987 Weiner et al.) “Inflatable Gastric Device for Treating Obesity and Method of Using the Same”, U.S. Pat. No. 5,074,868 (Dec. 24, 1991 Kuzmak) “Reversible Stoma-Adjustable Gastric Band”, U.S. Pat. No. 5,226,429 (Jul. 13, 1993 Kuzmak) “Laparoscopic Gastric Band and Method”, U.S. Pat. No. 5,234,454 (Aug. 10, 1993 Bangs) “Percutaneous Intragastric Balloon Catheter and Method for Controlling Body Weight Therewith”, U.S. Pat. No. 5,259,399 (Nov. 9, 1993 Brown) “Device and Method of Causing Weight Loss Using Removable Variable Volume Intragastric Bladder”, U.S. Pat. No. 5,449,368 (Sep. 12, 1995 Kuzmak) “Laparoscopic Adjustable Gastric Banding Device and Method for Implantation and Removal Thereof”, U.S. Pat. No. 5,601,604 (Feb. 11, 1997 Vincent) “Universal Gastric Band”, U.S. Pat. No. 5,868,141 (Feb. 9, 1999 Ellias) “Endoscopic Stomach Insert for Treating Obesity and Method for Use”, U.S. Pat. No. 5,993,473 (Nov. 30, 1999 Chan et al.) “Expandable Body Device for the Gastric Cavity and Method”, U.S. Pat. No. 6,067,991 (May 30, 2000 Forsell) “Mechanical Food Intake Restriction Device”, U.S. Pat. No. 6,454,785 (Sep. 24, 2002 De Hoyos Garza) “Percutaneous Intragastric Balloon Catheter for the Treatment Of Obesity”, U.S. Pat. No. 6,579,301 (Jun. 17, 2003 Bales et al.) “Intragastric Balloon Device Adapted to be Repeatedly Varied in Volume Without External Assistance”, U.S. Pat. No. 6,675,809 (Jan. 13, 2004 Stack et al.) “Satiation Devices and Methods”, U.S. Pat. No. 6,733,512 (May 11, 2004 Mcghan) “Self-Deflating Intragastric Balloon”, U.S. Pat. No. 6,981,980 (Jan. 3, 2006 Sampson et al.) “Self-Inflating Intragastric Volume-Occupying Device”, U.S. Pat. No. 7,033,373 (Apr. 25, 2006 DeLaTorre et al.) “Method and Device for Use in Minimally Invasive Placement of Space-Occupying Intragastric Devices”, U.S. Pat. No. 7,066,945 (Jun. 27, 2006 Hashiba et al.) “Intragastric Device for Treating Obesity”, and U.S. Pat. No. 7,112,186 (Sep. 26, 2006 Shah) “Gastro-Occlusive Device”.

Examples of prior art that appear to be best classified in this category also include: U.S. Pat. No. 7,354,454 (Apr. 8, 2008 Stack et al.) “Satiation Devices and Methods”, U.S. Pat. No. 7,470,251 (Dec. 30, 2008 Shah) “Gastro-Occlusive Device”, U.S. Pat. No. 7,682,306 (Mar. 23, 2010 Shah) “Therapeutic Intervention Systems Employing Implantable Balloon Devices”, U.S. Pat. No. 7,699,863 (Apr. 20, 2010 Marco et al.) “Bioerodible Self-Deployable Intragastric Implants”, U.S. Pat. No. 7,717,843 (May 18, 2010 Balbierz et al.) “Restrictive and/or Obstructive Implant for Inducing Weight Loss”, U.S. Pat. No. 7,758,493 (Jul. 20, 2010 Gingras) “Gastric Constriction Device”, U.S. Pat. No. 7,771,382 (Aug. 10, 2010 Levine et al.) “Resistive Anti-Obesity Devices”, U.S. Pat. No. 7,785,291 (Aug. 31, 2010 Marco et al.) “Bioerodible Self-Deployable Intragastric Implants”, U.S. Pat. No. 7,841,978 (Nov. 30, 2010 Gertner) “Methods and Devices for to Treatment of Obesity”, U.S. Pat. No. 7,963,907 (Jun. 21, 2011 Gertner) “Closed Loop Gastric Restriction Devices and Methods”, U.S. Pat. No. 8,001,974 (Aug. 23, 2011 Makower et al.) “Devices and Methods for Treatment of Obesity”, U.S. Pat. No. 8,016,744 (Sep. 13, 2011 Dlugos et al.) “External Pressure-Based Gastric Band Adjustment System and Method”, U.S. Pat. No. 8,016,745 (Sep. 13, 2011 Hassler et al.) “Monitoring of a Food Intake Restriction Device”, U.S. Pat. No. 8,029,455 (Oct. 4, 2011 Stack et al.) “Satiation Pouches and Methods of Use”, U.S. Pat. No. 8,048,169 (Nov. 1, 2011 Burnett et al.) “Pyloric Valve Obstructing Devices and Methods”, U.S. Pat. No. 8,066,780 (Nov. 29, 2011 Chen et al.) “Methods for Gastric Volume Control”, U.S. Pat. No. 8,083,756 (Dec. 27, 2011 Gannoe et al.) “Methods and Devices for Maintaining a Space Occupying Device in a Relatively Fixed Location Within a Stomach”, U.S. Pat. No. 8,083,757 (Dec. 27, 2011 Gannoe et al.) “Methods and Devices for Maintaining a Space Occupying Device in a Relatively Fixed Location Within a Stomach”, U.S. Pat. No. 8,142,469 (Mar. 27, 2012 Sosnowski et al.) “Gastric Space Filler Device, Delivery System, and Related Methods”, U.S. Pat. No. 8,142,513 (Mar. 27, 2012 Shalon et al.) “Devices and Methods for Altering Eating Behavior”, U.S. Pat. No. 8,187,297 (May 29, 2012 Makower et al.) “Devices and Methods for Treatment of Obesity”, U.S. Pat. No. 8,192,455 (Jun. 5, 2012 Brazzini et al.) “Compressive Device for Percutaneous Treatment of Obesity”, U.S. Pat. No. 8,202,291 (Jun. 19, 2012 Brister et al.) “Intragastric Device”, U.S. Pat. No. 8,226,593 (Jul. 24, 2012 Graham et al.) “Pyloric Valve”, U.S. Pat. No. 8,236,023 (Aug. 7, 2012 Birk et al.) “Apparatus and Method for Volume Adjustment of Intragastric Balloons”, U.S. Pat. No. 8,241,202 (Aug. 14, 2012 Balbierz et al.) “Restrictive and/or Obstructive Implant for Inducing Weight Loss”, U.S. Pat. No. 8,267,888 (Sep. 18, 2012 Marco et al.) “Bioerodible Self-Deployable Intragastric Implants”, U.S. Pat. No. 8,282,666 (Oct. 9, 2012 Birk) “Pressure Sensing Intragastric Balloon”, U.S. Pat. No. 8,292,911 (Oct. 23, 2012 Brister et al.) “Intragastric Device”, U.S. Pat. No. 8,292,911 (Oct. 23, 2012 Brister et al.) “Intragastric Device”, U.S. Pat. No. 8,295,932 (Oct. 23, 2012 Bitton et al.) “Ingestible Capsule for Appetite Regulation”, and U.S. Pat. No. 8,337,566 (Dec. 25, 2012 Stack et al.) “Method and Apparatus for Modifying the Exit Orifice of a Satiation Pouch”.

Examples of prior art that appear to be best classified in this category also include U.S. patent applications: 20010037127 (Nov. 1, 2001 De Hoyos Garza) “Percutaneous Intragastric Balloon Catheter for the Treatment of Obesity”, 20060252983 (Nov. 9, 2006 Lembo et al.) “Dynamically Adjustable Gastric Implants and Methods of Treating Obesity Using Dynamically Adjustable Gastric Implants”, 20060264699 (Nov. 23, 2006 Gertner) “Extragastric Minimally Invasive Methods and Devices to Treat Obesity”, 20070149994 (Jun. 28, 2007 Sosnowski et al.) “Intragastric Space Filler and Methods of Manufacture”, 20070207199 (Sep. 6, 2007 Sogin) “Appetite Suppression Device”, 20070276293 (Nov. 29, 2007 Gertner) “Closed Loop Gastric Restriction Devices and Methods”, 20070293885 (Dec. 20, 2007 Binmoeller) “Methods and Devices to Curb Appetite and/or to Reduce Food Intake”, 20080051824 (Feb. 28, 2008 Gertner) “Methods and Devices for to Treatment of Obesity”, 20080065168 (Mar. 13, 2008 Bitton et al.) “Ingestible Capsule for Appetite Regulation”, 20080147002 (Jun. 19, 2008 Gertner) “Obesity Treatment Systems”, 20080161717 (Jul. 3, 2008 Gertner) “Obesity Treatment Systems”, 20080188766 (Aug. 7, 2008 Gertner) “Obesity Treatment Systems”, 20080208240 (Aug. 28, 2008 Paz) “Implantable Device for Obesity Prevention”, 20080319471 (Dec. 25, 2008 Sosnowski et al.) “Gastric Space Filler Device, Delivery System, and Related Methods”, 20090131968 (May 21, 2009 Birk) “Pressure Sensing Intragastric Balloon”, 20090192535 (Jul. 30, 2009 Kasic) “Swallowable Self-Expanding Gastric Space Occupying Device”, 20090247992 (Oct. 1, 2009 Shalon et al.) “Devices and Methods for Altering Eating Behavior”, 20090259246 (Oct. 15, 2009 Eskaros et al.) “Intragastric Volume-Occupying Device”, 20090275973 (Nov. 5, 2009 Chen et al.) “Devices and Systems for Gastric Volume Control”, 20090306462 (Dec. 10, 2009 Lechner) “System for Controlling a Controllable Stomach Band”, 20100100117 (Apr. 22, 2010 Brister et al.) “Intragastric Device”, 20100114125 (May 6, 2010 Albrecht et al.) “Method of Remotely Adjusting a Satiation and Satiety-Inducing Implanted Device”, 20100114125 (May 6, 2010 Albrecht et al.) “Method of Remotely Adjusting a Satiation and Satiety-Inducing Implanted Device”, 20100130998 (May 27, 2010 Alverdy) “Balloon System and Methods for Treating Obesity”, 20100137897 (Jun. 3, 2010 Brister et al.) “Intragastric Device”, 20100152764 (Jun. 17, 2010 Merkle) “Device for Treating Obesity”, 20100286660 (Nov. 11, 2010 Gross) “Gastroretentive Duodenal Pill”, and 20100298632 (Nov. 25, 2010 Levine et al.) “Resistive Anti-Obesity Devices”.

Examples of prior art that appear to be best classified in this category also include U.S. patent applications: 20100312049 (Dec. 9, 2010 Forsell) “Apparatus for Treating Obesity”, 20100312050 (Dec. 9, 2010 Forsell) “Method and Instrument for Treating Obesity”, 20100312147 (Dec. 9, 2010 Gertner) “Obesity Treatment Systems”, 20100324361 (Dec. 23, 2010 Forsell) “Apparatus for Treating Obesity”, 20100331616 (Dec. 30, 2010 Forsell) “Method and Instrument for Treating Obesity”, 20100331617 (Dec. 30, 2010 Forsell) “Device, System and Method for Treating Obesity”, 20100332000 (Dec. 30, 2010 Forsell) “Device for Treating Obesity”, 20110009895 (Jan. 13, 2011 Gertner) “Methods and Devices to Treat Obesity”, 20110009896 (Jan. 13, 2011 Forsell) “Apparatus for Treating Obesity”, 20110015665 (Jan. 20, 2011 Marco et al.) “Bioerodible Self-Deployable Intragastric Implants”, 20110015666 (Jan. 20, 2011 Marco et al.) “Bioerodible Self-Deployable Intragastric Implants”, 20110022072 (Jan. 27, 2011 Marco et al.) “Bioerodible Self-Deployable Intragastric Implants”, 20110040318 (Feb. 17, 2011 Marco et al.) “Bioerodible Self-Deployable Intragastric Implants”, 20110060308 (Mar. 10, 2011 Stokes et al.) “Methods and Implants for Inducing Satiety in the Treatment of Obesity”, 20110060358 (Mar. 10, 2011 Stokes et al.) “Methods and Implants for Inducing Satiety in the Treatment of Obesity”, 20110092998 (Apr. 21, 2011 Hirszowicz et al.) “Balloon Hydraulic and Gaseous Expansion System”, 20110106129 (May 5, 2011 Gertner) “Methods and Devices to Treat Obesity”, 20110172693 (Jul. 14, 2011 Forsell) “Apparatus and Method for Treating Obesity”, 20110178544 (Jul. 21, 2011 Sosnowski et al.) “Gastric Space Filler Delivery System and Related Methods”, 20110196411 (Aug. 11, 2011 Forsell) “Apparatus for Treating Obesity”, 20110213448 (Sep. 1, 2011 Kim) “Apparatus and Methods for Minimally Invasive Obesity Treatment”, 20110213469 (Sep. 1, 2011 Chin et al.) “Systems and Methods for Bariatric Therapy”, 20110224714 (Sep. 15, 2011 Gertner) “Methods and Devices for the Surgical Creation of Satiety and Biofeedback Pathways”, 20110269711 (Nov. 3, 2011 Adden et al.) “Methods and Compositions for Inducing Satiety”, and 20110295056 (Dec. 1, 2011 Aldridge et al.) “Systems and Methods for Gastric Volume Regulation”.

Examples of prior art that appear to be best classified in this category also include U.S. patent applications: 20110295057 (Dec. 1, 2011 Aldridge et al.) “Systems and Methods for Gastric Volume Regulation”, 20110307075 (Dec. 15, 2011 Sharma) “Intragastric Device for Treating Obesity”, 20110319924 (Dec. 29, 2011 Cole et al.) “Gastric Space Occupier Systems and Methods of Use”, 20120004590 (Jan. 5, 2012 Stack et al.) “Satiation Pouches and Methods of Use”, 20120022322 (Jan. 26, 2012 Pasricha) “Methods and Devices for Treating Obesity”, 20120029550 (Feb. 2, 2012 Forsell) “Obesity Treatment”, 20120041463 (Feb. 16, 2012 Forsell) “Obesity Treatment”, 20120053613 (Mar. 1, 2012 Weitzner et al.) “Gastric Filler Devices for Obesity Therapy”, 20120089168 (Apr. 12, 2012 Baker et al.) “Bariatric Device and Method”, 20120089170 (Apr. 12, 2012 Dominguez) “Intragastric Balloon Geometries”, 20120089172 (Apr. 12, 2012 Babkes et al.) “Re-Shaping Intragastric Implants”, 20120095384 (Apr. 19, 2012 Babkes et al.) “Stomach-Spanning Gastric Implants”, 20120095492 (Apr. 19, 2012 Babkes et al.) “Variable Size Intragastric Implant Devices”, 20120095494 (Apr. 19, 2012 Dominguez et al.) “Intragastric Implants with Collapsible Frames”, 20120095495 (Apr. 19, 2012 Babkes et al.) “Space-Filling Intragastric Implants with Fluid Flow”, 20120095496 (Apr. 19, 2012 Dominguez et al.) “Reactive Intragastric Implant Devices”, 20120095497 (Apr. 19, 2012 Babkes et al.) “Non-Inflatable Gastric Implants and Systems”, 20120095499 (Apr. 19, 2012 Babkes et al.) “Upper Stomach Gastric Implants”, 20120123465 (May 17, 2012 Nihalani) “Method and Apparatus for Treating Obesity and Controlling Weight Gain using Self-Expanding Intragastric Devices”, 20120150316 (Jun. 14, 2012 Carvalho) “Esophageal Flow Controller”, 20120165855 (Jun. 28, 2012 Shalon et al.) “Devices and Methods for Altering Eating Behavior”, 20120165855 (Jun. 28, 2012 Shalon et al.) “Devices and Methods for Altering Eating Behavior”, 20120191123 (Jul. 26, 2012 Brister et al.) “Intragastric Device”, and 20120191124 (Jul. 26, 2012 Brister et al.) “Intragastric Device”.

Examples of prior art that appear to be best classified in this category also include U.S. patent applications: 20120191125 (Jul. 26, 2012 Babkes et al.) “Intragastric Implants with Multiple Fluid Chambers”, 20120191126 (Jul. 26, 2012 Pecor et al.) “Inflation and Deflation Mechanisms for Inflatable Medical Devices”, 20120203061 (Aug. 9, 2012 Birk) “Bariatric Device and Method for Weight Loss”, 20120215249 (Aug. 23, 2012 Brazzini et al.) “Compressive Device for Percutaneous Treatment of Obesity”, 20120221037 (Aug. 30, 2012 Birk et al.) “Bariatric Device and Method for Weight Loss”, 20120232576 (Sep. 13, 2012 Brister et al.) “Intragastric Device”, 20120232577 (Sep. 13, 2012 Birk et al.) “Bariatric Device and Method for Weight Loss”, 20120253378 (Oct. 4, 2012 Makower et al.) “Devices and Methods for Treatment of Obesity”, 20120259427 (Oct. 11, 2012 Graham et al.) “Pyloric Valve”, 20120265030 (Oct. 18, 2012 Li) “Devices Systems Kits and Methods for Treatment of Obesity”, 20120265234 (Oct. 18, 2012 Brister et al.) “Intragastric Device”, 20120283766 (Nov. 8, 2012 Makower et al.) “Devices and Methods for Treatment of Obesity”, 20120289992 (Nov. 15, 2012 Quijano et al.) “Intragastric Balloon System and Therapeutic Processes and Products”, and 20120316387 (Dec. 13, 2012 Volker) “Adjustable Gastric Wrap (AGW)”.

32. Gastrointestinal (GI) Volume or Pressure or Flow Modification (with Drug)

Prior art in this category is similar to that in the previous category, except that it also includes delivery of a pharmaceutical agent. In various examples, this category can include drug-eluting gastric balloons, gastric balloons with an integral drug pump, and drug-eluting gastric stents. Although drug delivery can provide another therapeutic modality for these devices, the addition of drug delivery does not correct most of the potential limitations of devices that were discussed in the previous category. Accordingly, most of these limitations still apply to devices in this present category.

Examples of prior art that appear to be best classified in this category include: U.S. patents U.S. Pat. No. 6,627,206 (Sep. 30, 2003 Lloyd) “Method and Apparatus for Treating Obesity and for Delivering Time-Released Medicaments”, U.S. Pat. No. 7,121,283 (Oct. 17, 2006 Stack et al.) “Satiation Devices and Methods”, U.S. Pat. No. 7,152,607 (Dec. 26, 2006 Stack et al.) “Satiation Devices and Methods”, U.S. Pat. No. 7,833,280 (Nov. 16, 2010 Stack et al.) “Satiation Devices and Methods”, U.S. Pat. No. 7,854,745 (Dec. 21, 2010 Brister et al.) “Intragastric Device”, U.S. Pat. No. 8,070,768 (Dec. 6, 2011 Kim et al.) “Devices and Methods for Treatment of Obesity”, U.S. Pat. No. 8,162,969 (Apr. 24, 2012 Brister et al.) “Intragastric Device”, U.S. Pat. No. 8,177,853 (May 15, 2012 Stack et al.) “Satiation Devices and Methods”, and U.S. Pat. No. 8,226,602 (Jul. 24, 2012 Quijana et al.) “Intragastric Balloon System and Therapeutic Processes and Products”; and U.S. patent applications 20030021822 (Jan. 30, 2003 Lloyd) “Method and Apparatus for Treating Obesity and for Delivering Time-Released Medicaments”, 20040172142 (Sep. 2, 2004 Stack et al.) “Satiation Devices and Methods”, 20070265598 (Nov. 15, 2007 Karasik) “Device and Method for Treating Weight Disorders”, 20080243071 (Oct. 2, 2008 Quijano et al.) “Intragastric Balloon System and Therapeutic Processes and Products”, 20100100116 (Apr. 22, 2010 Brister et al.) “Intragastric Volume-Occupying Device and Method for Fabricating Same”, 20100114150 (May 6, 2010 Magal) “Duodenal Stimulation Devices and Methods for the Treatment of Conditions Relating to Eating Disorders”, 20120016287 (Jan. 19, 2012 Stack et al.) “Satiation Devices and Methods”, 20120022430 (Jan. 26, 2012 Stack et al.) “Satiation Devices and Methods”, 20120245553 (Sep. 27, 2012 Raven et al.) “Intragastric Volume Occupying Device with Active Agents”, and 20120271217 (Oct. 25, 2012 Stack et al.) “Satiation Devices and Methods”.

### 33. Gastrointestinal (GI) Sleeve or Liner

Prior art in this category includes gastrointestinal sleeves, gastrointestinal liners, and other flexible tubular devices that are implanted within a person's gastrointestinal tract to reduce absorption of nutrients from food by reducing contact between food and the walls of the gastrointestinal tract. Gastric sleeves are common examples of devices in this category. As long as devices in this category can be securely and safely fastened to their proper location within the gastrointestinal tract so that they do not migrate or cause blockages, these devices have potential to be a useful addition to the available approaches to limiting absorption of nutrients from food. Most are less invasive than gastric bypass operations and can be removed if they do not work well.

However, gastrointestinal sleeves and liners in the prior art are food blind. They are not able to selectively reduce absorption of nutrients from unhealthy food and allow normal absorption of nutrients from healthy food. Also, they are implanted and thus do require an operation. In this respect, they are more invasive than purely-external approaches to monitoring and modifying food consumption.

Examples of prior art that appear to be best classified in this category include: U.S. Pat. No. 4,641,653 (Feb. 10, 1987 Rockey) “Medical Sleeve”, U.S. Pat. No. 7,220,284 (May 22, 2007 Kagan et al.) “Gastrointestinal Sleeve Device and Methods for Treatment of Morbid Obesity”, U.S. Pat. No. 7,695,446 (Apr. 13, 2010 Levine et al.) “Methods of Treatment Using a Bariatric Sleeve”, U.S. Pat. No. 7,753,870 (Jul. 13, 2010 Demarais et al.) “Systems and Methods for Treating Obesity”, U.S. Pat. No. 7,794,447 (Sep. 14, 2010 Dann et al.) “Gastrointestinal Sleeve Device and Methods for Treatment of Morbid Obesity”, U.S. Pat. No. 7,837,643 (Nov. 23, 2010 Levine et al.) “Methods and Devices for Placing a Gastrointestinal Sleeve”, U.S. Pat. No. 7,837,669 (Nov. 23, 2010 Dann et al.) “Devices and Methods for Endolumenal Gastrointestinal Bypass”, U.S. Pat. No. 7,846,138 (Dec. 7, 2010 Dann et al.) “Cuff and Sleeve System for Gastrointestinal Bypass”, U.S. Pat. No. 7,935,073 (May 3, 2011 Levine et al.) “Methods of Treatment Using a Bariatric Sleeve”, U.S. Pat. No. 7,981,162 (Jul. 19, 2011 Stack et al.) “Satiation Devices and Methods”, U.S. Pat. No. 8,012,140 (Sep. 6, 2011 Kagan et al.) “Methods of Transmural Attachment in the Gastrointestinal System”, U.S. Pat. No. 8,057,420 (Nov. 15, 2011 Meade et al.) “Gastrointestinal Implant with Drawstring”, U.S. Pat. No. 8,070,743 (Dec. 6, 2011 Kagan et al.) “Devices and Methods for Attaching an Endolumenal Gastrointestinal Implant”, U.S. Pat. No. 8,109,895 (Feb. 7, 2012 Williams et al.) “Intestinal Sleeves and Associated Deployment Systems and Methods”, U.S. Pat. No. 8,137,301 (Mar. 20, 2012 Levine et al.) “Bariatric Sleeve”, U.S. Pat. No. 8,162,871 (Apr. 24, 2012 Levine et al.) “Bariatric Sleeve”, U.S. Pat. No. 8,182,459 (May 22, 2012 Dann et al.) “Devices and Methods for Endolumenal Gastrointestinal Bypass”, U.S. Pat. No. 8,211,186 (Jul. 3, 2012 Belhe et al.) “Modular Gastrointestinal Prostheses”, U.S. Pat. No. 8,216,158 (Jul. 10, 2012 Johnson) “Implantation of a Medical Device Within a Lumen”, U.S. Pat. No. 8,282,598 (Oct. 9, 2012 Belhe et al.) “External Anchoring Configurations for Modular Gastrointestinal Prostheses”, and U.S. Pat. No. 8,303,669 (Nov. 6, 2012 Meade et al.) “Methods and Apparatus for Anchoring within the Gastrointestinal Tract”.

Examples of prior art that appear to be best classified in this category also include U.S. patent applications: 20090093767 (Apr. 9, 2009 Kelleher) “Devices and Methods for Endolumenal Therapy”, 20090240340 (Sep. 24, 2009 Levine et al.) “Bariatric Sleeve”, 20090248171 (Oct. 1, 2009 Levine et al.) “Bariatric Sleeve”, 20100256775 (Oct. 7, 2010 Belhe et al.) “Modular Gastrointestinal Prostheses”, 20100298631 (Nov. 25, 2010 Stack et al.) “Satiation Devices and Methods”, 20110009690 (Jan. 13, 2011 Belhe et al.) “External Anchoring Configurations for Modular Gastrointestinal Prostheses”, 20110087146 (Apr. 14, 2011 Ryan et al.) “Stomach Bypass for the Treatment of Obesity”, 20110106273 (May 5, 2011 Belhe et al.) “Gastrointestinal Prostheses Having Partial Bypass Configurations”, 20110245752 (Oct. 6, 2011 Levine et al.) “Methods of Treatment Using a Bariatric Sleeve”, 20110270410 (Nov. 3, 2011 Stack et al.) “Satiation Devices and Methods”, 20120004676 (Jan. 5, 2012 Vargas) “Intragastric Implant Devices”, 20120041465 (Feb. 16, 2012 Shalon) “Devices and Methods for Treating Gastrointestinal and Metabolic Disorders”, 20120053504 (Mar. 1, 2012 Kagan et al.) “Methods for Attachment of a Gastrointestinal Sleeve”, 20120065571 (Mar. 15, 2012 Thompson et al.) “Expandable Pyloric Anchors and Methods for Securing Intestinal Bypass Sleeves”, 20120116286 (May 10, 2012 Williams et al.) “Intestinal Sleeves and Associated Deployment Systems and Methods”, 20120184893 (Jul. 19, 2012 Thompson et al.) “Anchors and Methods for Intestinal Bypass Sleeves”, 20120215152 (Aug. 23, 2012 Levine et al.) “Bariatric Sleeve”, 20120232459 (Sep. 13, 2012 Dann et al.) “Devices and Methods for Endolumenal Gastrointestinal Bypass”, 20120253259 (Oct. 4, 2012 Belhe et al.) “Modular Gastrointestinal Prostheses”, and 20120253260 (Oct. 4, 2012 Belhe et al.) “Gastrointestinal Prostheses”.

34. Gastrointestinal (GI) Sleeve or Liner (with Drug)

Prior art in this category is similar to that in the previous category, except that it also includes delivery of a pharmaceutical agent. In various examples, this category includes drug-eluting gastric sleeves and liners. Although drug delivery can provide a secondary therapeutic modality for these devices, the addition of drug delivery does not help differentiate between healthy and unhealthy food. Accordingly, these devices remain food blind. They are not able to selectively reduce absorption of nutrients from unhealthy food and allow normal absorption of nutrients from healthy food.

Examples of prior art that appear to be best classified in this category include U.S. patent applications 20110040232 (Feb. 17, 2011 Magal) “Duodenal Liner Device” and 20120232460 (Sep. 13, 2012 Raven et al.) “Intraluminal Sleeve with Active Agents”.

### 35. Electrical Stimulation (General)

Prior art in this category includes implantable devices that deliver electromagnetic energy to a portion of a person's gastrointestinal tract or to a nerve that innervates a portion of the person's gastrointestinal tract. In an example, electrical stimulation can be applied directly to a person's stomach in order to induce a sense of satiety and/or modify gastric motility. The intent of such gastric stimulation is to reduce a person's food consumption. In another example, electrical energy can be applied to block normal neural transmissions in a nerve that innervates a person's stomach in order to reduce gastric functioning and thereby reduce food consumption. This category of art has considerable potential (no pun intended) to modify food consumption. It is relatively non-invasive with respect to other internal procedures, is adjustable, and is reversible.

In order for devices in this category to be successful in modifying food consumption, the gastrointestinal organ or nerve to which electrical energy is applied must not accommodate (ie: become inured to) the application of electrical energy. If an organ or nerve does accommodate the application of electrical energy, then the organ or nerve stops responding to the applied energy in a therapeutic manner. For this reason, devices in this category generally apply electrical energy in a non-continuous manner.

The ability to differentiate between consumption of healthy vs unhealthy food could enable such devices to selectively deliver electrical energy only when a person eats unhealthy food. This differentiating ability would allow use of higher power levels without the problem of accommodation and make such devices more effective for modifying food consumption. Such ability could also encourage the person to have a healthier diet and extend a device's battery life. However, prior art devices in this category do not appear to offer the ability to differentiate between consumption of healthy vs unhealthy food.

Examples of prior art that appear to be best classified in this category include: U.S. Pat. No. 3,411,507 (Nov. 19, 1968 Wingrove) “Method of Gastrointestinal Stimulation with Electrical Pulses”, U.S. Pat. No. 5,188,104 (Feb. 23, 1993 Wernicke et al.) “Treatment of Eating Disorders by Nerve Stimulation”, U.S. Pat. No. 5,423,872 (Jun. 13, 1995 Cigaina) “Process and Device for Treating Obesity and Syndromes Related to Motor Disorders of the Stomach of a Patient”, U.S. Pat. No. 5,690,691 (Nov. 25, 1997 Chen et al.) “Gastro-Intestinal Pacemaker Having Phased Multi-Point Stimulation”, U.S. Pat. No. 5,716,385 (Feb. 10, 1998 Mittal et al.) “Crural Diaphragm Pacemaker and Method for Treating Esophageal Reflux Disease (Mittal)”, U.S. Pat. No. 5,891,185 (Apr. 6, 1999 Freed et al.) “Method and Apparatus for Treating Oropharyngeal Disorders with Electrical Stimulation”, U.S. Pat. No. 6,091,992 (Jul. 18, 2000 Bourgeois et al.) “Method and Apparatus for Electrical Stimulation of the Gastrointestinal Tract”, U.S. Pat. No. 6,243,607 (Jun. 5, 2001 Mintchev et al.) “Gastro-Intestinal Electrical Pacemaker”, U.S. Pat. No. 6,564,101 (May 13, 2003 Zikria) “Electrical System for Weight Loss and Laparoscopic Implanation Thereof”, U.S. Pat. No. 6,587,719 (Jul. 1, 2003 Barrett et al.) “Treatment of Obesity by Bilateral Vagus Nerve Stimulation”, U.S. Pat. No. 6,609,025 (Aug. 19, 2003 Barrett et al.) “Treatment of Obesity by Bilateral Sub-Diaphragmatic Nerve Stimulation”, U.S. Pat. No. 6,684,104 (Jan. 27, 2004 Gordon et al.) “Gastric Stimulator Apparatus and Method for Installing”, U.S. Pat. No. 6,760,626 (Jul. 6, 2004 Boveja) “Apparatus and Method for Treatment of Neurological and Neuropsychiatric Disorders Using Programmerless Implantable Pulse Generator System”, U.S. Pat. No. 6,879,859 (Apr. 12, 2005 Boveja) “External Pulse Generator for Adjunct (Add-On) Treatment of Obesity Eating Disorders Neurological Neuropsychiatric and Urological Disorders”, U.S. Pat. No. 7,072,720 (Jul. 4, 2006 Puskas) “Devices and Methods for Vagus Nerve Stimulation”, U.S. Pat. No. 7,167,750 (Jan. 23, 2007 Knudson et al.) “Obesity Treatment with Electrically Induced Vagal Down Regulation”, U.S. Pat. No. 7,177,693 (Feb. 13, 2007 Starkebaum) “Gastric Stimulation for Altered Perception to Treat Obesity”, and U.S. Pat. No. 7,236,822 (Jun. 26, 2007 Dobak) “Wireless Electric Modulation of Sympathetic Nervous System”.

Examples of prior art that appear to be best classified in this category also include: U.S. Pat. No. 7,239,912 (Jul. 3, 2007 Dobak) “Electric Modulation of Sympathetic Nervous System”, U.S. Pat. No. 7,299,091 (Nov. 20, 2007 Barrett et al.) “Treatment of Obesity by Bilateral Vagus Nerve Stimulation”, U.S. Pat. No. 7,529,582 (May 5, 2009 Dilorenzo) “Method and Apparatus for Neuromodulation and Physiologic Modulation for the Treatment of Metabolic and Neuropsychiatric Disease”, U.S. Pat. No. 7,551,964 (Jun. 23, 2009 Dobak) “Splanchnic Nerve Stimulation for Treatment of Obesity”, U.S. Pat. No. 7,580,751 (Aug. 25, 2009 Starkebaum) “Intra-Luminal Device for Gastrointestinal Stimulation”, U.S. Pat. No. 7,599,736 (Oct. 6, 2009 Dilorenzo) “Method and Apparatus for Neuromodulation and Physiologic Modulation for the Treatment of Metabolic and Neuropsychiatric Disease”, U.S. Pat. No. 7,657,310 (Feb. 2, 2010 Buras) “Treatment of Reproductive Endocrine Disorders by Vagus Nerve Stimulation”, U.S. Pat. No. 7,664,551 (Feb. 16, 2010 Cigaina) “Treatment of the Autonomic Nervous System”, U.S. Pat. No. 7,689,276 (Mar. 30, 2010 Dobak) “Dynamic Nerve Stimulation for Treatment of Disorders”, U.S. Pat. No. 7,689,277 (Mar. 30, 2010 Dobak) “Neural Stimulation for Treatment of Metabolic Syndrome and Type 2 Diabetes”, U.S. Pat. No. 7,702,386 (Apr. 20, 2010 Dobak et al.) “Nerve Stimulation for Treatment of Obesity Metabolic Syndrome and Type 2 Diabetes”, U.S. Pat. No. 7,729,771 (Jun. 1, 2010 Knudson et al.) “Nerve Stimulation and Blocking for Treatment of Gastrointestinal Disorders”, U.S. Pat. No. 7,756,582 (Jul. 13, 2010 Imran et al.) “Gastric Stimulation Anchor and Method”, U.S. Pat. No. 7,840,278 (Nov. 23, 2010 Puskas) “Devices and Methods for Vagus Nerve Stimulation”, U.S. Pat. No. 7,945,323 (May 17, 2011 Jaax et al.) “Treatment of Obesity and/or Type II Diabetes by Stimulation of the Pituitary Gland”, U.S. Pat. No. 7,979,127 (Jul. 12, 2011 Imran) “Digestive Organ Retention Device”, U.S. Pat. No. 7,986,995 (Jul. 26, 2011 Knudson et al.) “Bulimia Treatment”, U.S. Pat. No. 8,082,039 (Dec. 20, 2011 Kim et al.) “Stimulation Systems”, U.S. Pat. No. 8,145,299 (Mar. 27, 2012 Dobak) “Neural Stimulation for Treatment of Metabolic Syndrome and Type 2 Diabetes”, U.S. Pat. No. 8,150,508 (Apr. 3, 2012 Craig) “Vagus Nerve Stimulation Method”, U.S. Pat. No. 8,280,505 (Oct. 2, 2012 Craig) “Vagus Nerve Stimulation Method”, U.S. Pat. No. 8,301,256 (Oct. 30, 2012 Policker et al.) “GI Lead Implantation”, and U.S. Pat. No. 8,340,772 (Dec. 25, 2012 Vase et al.) “Brown Adipose Tissue Utilization Through Neuromodulation”.

Examples of prior art that appear to be best classified in this category also include U.S. patent applications: 20040167583 (Aug. 26, 2004 Knudson et al.) “Electrode Band Apparatus and Method”, 20070027498 (Feb. 1, 2007 Maschino et al.) “Selective Nerve Stimulation for the Treatment of Eating Disorders”, 20070135846 (Jun. 14, 2007 Knudson et al.) “Vagal Obesity Treatment”, 20070150021 (Jun. 28, 2007 Chen et al.) “Gastrointestinal Electrical Stimulation”, 20070203521 (Aug. 30, 2007 Dobak et al.) “Nerve Stimulation for Treatment of Obesity Metabolic Syndrome and Type 2 Diabetes”, 20080046013 (Feb. 21, 2008 Lozano) “Method for Treating Eating Disorders”, 20080183238 (Jul. 31, 2008 Chen) “Process for Electrostimulation Treatment of Morbid Obesity”, 20080195171 (Aug. 14, 2008 Sharma) “Method and Apparatus for Electrical Stimulation of the Pancreatico-Biliary System”, 20090018606 (Jan. 15, 2009 Sparks et al.) “Methods and Devices for Stimulation of an Organ with the Use of a Transectionally Placed Guide Wire”, 20090259274 (Oct. 15, 2009 Simon et al.) “Methods and Apparatus for Electrical Treatment Using Balloon and Electrode”, 20090259279 (Oct. 15, 2009 Dobak) “Splanchnic Nerve Stimulation for Treatment of Obesity”, 20100087706 (Apr. 8, 2010 Syed et al.) “Lead Access”, 20100094375 (Apr. 15, 2010 Donders et al.) “Neural Electrode Treatment”, 20100168815 (Jul. 1, 2010 Knudson et al.) “Nerve Stimulation and Blocking for Treatment of Gastrointestinal Disorders”, 20100183700 (Jul. 22, 2010 Stojanovic-Susulic et al.) “Implantable Pump for Protein Delivery for Obesity Control by Drug Infusion into the Brain”, 20100234917 (Sep. 16, 2010 Imran) “Digestive Organ Retention Device”, and 20100286745 (Nov. 11, 2010 Imran) “Radially Expandable Gastrointestinal Stimulation Device”.

Examples of prior art that appear to be best classified in this category also include U.S. patent applications: 20110034967 (Feb. 10, 2011 Chen et al.) “Gastrointestinal Electrical Stimulation”, 20110034968 (Feb. 10, 2011 Knudson et al.) “Controlled Vagal Blockage Therapy”, 20110166582 (Jul. 7, 2011 Syed et al.) “Endoscopic Device Delivery System”, 20110230938 (Sep. 22, 2011 Simon et al.) “Device and Methods for Non-Invasive Electrical Stimulation and Their Use for Vagal Nerve Stimulation”, 20110238035 (Sep. 29, 2011 Jaax et al.) “Treatment of Obesity and/or Type II Diabetes by Stimulation of the Pituitary Gland”, 20110270344 (Nov. 3, 2011 Knudson et al.) “Bulimia Treatment”, 20110307023 (Dec. 15, 2011 Tweden et al.) “Neural Modulation Devices and Methods”, 20110319969 (Dec. 29, 2011 Dobak) “Electric Modulation of Sympathetic Nervous System”, 20120041509 (Feb. 16, 2012 Knudson et al.) “Controlled Vagal Blockage Therapy”, 20120053653 (Mar. 1, 2012 Hiernaux et al.) “Gastrointestinal Device”, 20120053660 (Mar. 1, 2012 Dobak) “Splanchnic Nerve Stimulation for Treatment of Obesity”, 20120071947 (Mar. 22, 2012 Gupta et al.) “Method and Apparatus for Event-Triggered Reinforcement of a Favorable Brain State”, 20120143279 (Jun. 7, 2012 Ekchian et al.) “Methods and Kits for Treating Appetite Suppressing Disorders and Disorders with an Increased Metabolic Rate”, 20120209354 (Aug. 16, 2012 Raykhman) “System and Methods for Producing and Delivering Electrical Impulses”, and 20120310295 (Dec. 6, 2012 Libbus et al.) “Systems and Methods for Avoiding Neural Stimulation Habituation”.

36. Electrical Stimulation (with Glucose Sensor)

Devices in this category are similar to devices in the previous category of general electrical stimulation except that they also include a glucose sensor. They deliver electromagnetic energy to person's gastrointestinal tract or to a nerve that innervates their gastrointestinal tract. In an example, a person's blood glucose level can be monitored and gastrointestinal electrical stimulation can be triggered when the person's glucose level indicates that such stimulation is most needed. Selective electrical stimulation can help to target therapeutic benefit.

Examples of prior art that appear to be best classified in this category include: U.S. Pat. No. 6,093,167 (Jul. 25, 2000 Houben et al.) “System for Pancreatic Stimulation and Glucose Measurement”, U.S. Pat. No. 6,185,452 (Feb. 6, 2001 Schulman et al.) “Battery-Powered Patient Implantable Device”, U.S. Pat. No. 6,571,127 (May 27, 2003 Ben-Haim et al.) “Method of Increasing the Motility of a GI Tract”, U.S. Pat. No. 6,600,953 (Jul. 29, 2003 Flesler et al.) “Acute and Chronic Electrical Signal Therapy for Obesity”, U.S. Pat. No. 6,832,114 (Dec. 14, 2004 Whitehurst et al.) “Systems and Methods for Modulation of Pancreatic Endocrine Secretion and Treatment of Diabetes”, U.S. Pat. No. 6,922,590 (Jul. 26, 2005 Whitehurst) “Systems and Methods for Treatment of Diabetes by Electrical Brain Stimulation and/or Drug Infusion”, U.S. Pat. No. 6,993,391 (Jan. 31, 2006 Flesler et al.) “Acute and Chronic Electrical Signal Therapy for Obesity”, U.S. Pat. No. 7,020,531 (Mar. 28, 2006 Colliou et al.) “Gastric Device and Suction Assisted Method for Implanting a Device on a Stomach Wall”, U.S. Pat. No. 7,440,806 (Oct. 21, 2008 Whitehurst et al.) “Systems and Methods for Treatment of Diabetes by Electrical Brain Stimulation and/or Drug Infusion”, U.S. Pat. No. 7,477,944 (Jan. 13, 2009 Whitehurst et al.) “Systems and Methods for Modulation of Pancreatic Endocrine Secretion and Treatment of Diabetes”, U.S. Pat. No. 7,502,649 (Mar. 10, 2009 Ben-Haim et al.) “Gastrointestinal Methods and Apparatus for Use in Treating Disorders”, U.S. Pat. No. 7,512,442 (Mar. 31, 2009 Flesler et al.) “Acute and Chronic Electrical Signal Therapy for Obesity”, U.S. Pat. No. 7,558,629 (Jul. 7, 2009 Keimel et al.) “Energy Balance Therapy for Obesity Management”, U.S. Pat. No. 7,937,145 (May 3, 2011 Dobak) “Dynamic Nerve Stimulation Employing Frequency Modulation”, U.S. Pat. No. 8,019,421 (Sep. 13, 2011 Darvish et al.) “Blood Glucose Level Control”, U.S. Pat. No. 8,095,218 (Jan. 10, 2012 Gross et al.) “GI and Pancreatic Device for Treating Obesity and Diabetes”, U.S. Pat. No. 8,135,470 (Mar. 13, 2012 Keimel et al.) “Energy Balance Therapy for Obesity Management”, U.S. Pat. No. 8,209,037 (Jun. 26, 2012 Laufer et al.) “Methods and Devices for Medical Treatment”, U.S. Pat. No. 8,321,030 (Nov. 27, 2012 Maniak et al.) “Esophageal Activity Modulated Obesity Therapy”, U.S. Pat. No. 8,321,030 (Nov. 27, 2012 Maniak et al.) “Esophageal Activity Modulated Obesity Therapy”, and U.S. Pat. No. 8,346,363 (Jan. 1, 2013 Darvish et al.) “Blood Glucose Level Control”.

Examples of prior art that appear to be best classified in this category also include U.S. patent applications: 20040044376 (Mar. 4, 2004 Flesler et al.) “Acute and Chronic Electrical Signal Therapy for Obesity”, 20050149142 (Jul. 7, 2005 Starkebaum) “Gastric Stimulation Responsive to Sensing Feedback”, 20050222638 (Oct. 6, 2005 Foley et al.) “Sensor Based Gastrointestinal Electrical Stimulation for the Treatment of Obesity or Motility Disorders”, 20060074459 (Apr. 6, 2006 Flesler et al.) “Acute and Chronic Electrical Signal Therapy for Obesity”, 20070016262 (Jan. 18, 2007 Gross et al.) “GI and Pancreatic Device for Treating Obesity and Diabetes”, 20070027493 (Feb. 1, 2007 Ben-Haim et al.) “Gastrointestinal Methods and Apparatus for Use in Treating Disorders and Controlling Blood Sugar”, 20070179556 (Aug. 2, 2007 Ben-Haim et al.) “Gastrointestinal Methods and Apparatus for Use in Treating Disorders”, 20070255334 (Nov. 1, 2007 Keimel et al.) “Energy Balance Therapy for Obesity Management”, 20090018594 (Jan. 15, 2009 Laufer et al.) “Methods and Devices for Medical Treatment”, 20090030474 (Jan. 29, 2009 Brynelsen et al.) “Sensor Driven Gastric Stimulation for Patient Management”, 20090062881 (Mar. 5, 2009 Gross et al.) “GI and Pancreatic Device for Treating Obesity and Diabetes”, 20090088816 (Apr. 2, 2009 Harel et al.) “Gastrointestinal Methods and Apparatus for Use in Treating Disorders and Controlling Blood Sugar”, 20090240194 (Sep. 24, 2009 Keimel et al.) “Energy Balance Therapy for Obesity Management”, 20100268306 (Oct. 21, 2010 Maniak et al.) “Esophageal Activity Modulated Obesity Therapy”, 20110087076 (Apr. 14, 2011 Brynelsen et al.) “Feedback Systems and Methods for Communicating Diagnostic and/or Treatment Signals to Enhance Obesity Treatments”, 20120083855 (Apr. 5, 2012 Gross et al.) “GI and Pancreatic Device for Treating Obesity and Diabetes”, 20120214140 (Aug. 23, 2012 Brynelsen et al.) “Feedback Systems and Methods for Communicating Diagnostic and/or Treatment Signals to Enhance Obesity Treatments”, 20120259389 (Oct. 11, 2012 Starkebaum et al.) “Treatment of Postprandial Hyperglycemia by Gastric Electrical Stimulation”, and 20120323099 (Dec. 20, 2012 Mothilal et al.) “Implantable Medical Device Electrode Assembly”.

37. Electrical Stimulation (with General Sensor)

Devices in this category are similar to devices in the prior category of general electrical stimulation except that they also include one or more sensors other than a glucose sensor. Like devices in prior categories, they deliver electromagnetic energy to person's gastrointestinal tract or to a nerve that innervates their gastrointestinal tract. In an example, the electromagnetic properties of a person's esophagus or stomach can be monitored by an electromagnetic sensor and gastrointestinal electrical stimulation can be triggered when the sensor indicates that a person is consuming food. Selective electrical stimulation can help to target therapeutic benefit.

Examples of prior art that appear to be best classified in this category include: U.S. Pat. No. 5,263,480 (Nov. 23, 1993 Wernicke et al.) “Treatment of Eating Disorders by Nerve Stimulation”, U.S. Pat. No. 5,292,344 (Mar. 8, 1994 Douglas) “Percutaneously Placed Electrical Gastrointestinal Pacemaker Stimulatory System, Sensing System, and PH Monitoring System, with Optional Delivery Port”, U.S. Pat. No. 5,540,730 (Jul. 30, 1996 Terry et al.) “Treatment of Motility Disorders by Nerve Stimulation”, U.S. Pat. No. 5,836,994 (Nov. 17, 1998 Bourgeois) “Method and Apparatus for Electrical Stimulation of the Gastrointestinal Tract”, U.S. Pat. No. 5,861,014 (Jan. 19, 1999 Familoni) “Method and Apparatus for Sensing a Stimulating Gastrointestinal Tract On-Demand”, U.S. Pat. No. 5,995,872 (Nov. 30, 1999 Bourgeois) “Method and Apparatus for Electrical Stimulation of the Gastrointestinal Tract”, U.S. Pat. No. 6,083,249 (Jul. 4, 2000 Familoni) “Apparatus for Sensing and Stimulating Gastrointestinal Tract On-Demand”, U.S. Pat. No. 6,104,955 (Aug. 15, 2000 Bourgeois) “Method and Apparatus for Electrical Stimulation of the Gastrointestinal Tract”, U.S. Pat. No. 6,115,635 (Sep. 5, 2000 Bourgeois) “Method and Apparatus for Electrical Stimulation of the Gastrointestinal Tract”, U.S. Pat. No. 6,216,039 (Apr. 10, 2001 Bourgeois) “Method and Apparatus for Treating Irregular Gastric Rhythms”, U.S. Pat. No. 6,327,503 (Dec. 4, 2001 Familoni) “Method and Apparatus for Sensing and Stimulating Gastrointestinal Tract On-Demand”, U.S. Pat. No. 6,535,764 (Mar. 18, 2003 Imran et al.) “Gastric Treatment and Diagnosis Device and Method (Intrapace: Imran)”, U.S. Pat. No. 6,591,137 (Jul. 8, 2003 Fischell et al.) “Implantable Neuromuscular Stimulator for the Treatment of Gastrointestinal Disorders”, and U.S. Pat. No. 6,735,477 (May 11, 2004 Levine) “Internal Monitoring System with Detection of Food Intake”.

Examples of prior art that appear to be best classified in this category also include: U.S. Pat. No. 6,826,428 (Nov. 30, 2004 Chen et al.) “Gastrointestinal Electrical Stimulation”, U.S. Pat. No. 6,993,391 (Jan. 31, 2006 Flesler et al.) “Acute and Chronic Electrical Signal Therapy for Obesity”, U.S. Pat. No. 7,054,690 (May 30, 2006 Imran) “Gastrointestinal Stimulation Device”, U.S. Pat. No. 7,120,498 (Oct. 10, 2006 Imran et al.) “Method and Device for Securing a Functional Device to a Stomach”, U.S. Pat. No. 7,430,450 (Sep. 30, 2008 Imran) “Device and Method for Treating Obesity”, U.S. Pat. No. 7,437,195 (Oct. 14, 2008 Policker et al.) “Regulation of Eating Habits”, U.S. Pat. No. 7,509,174 (Mar. 24, 2009 Imran et al.) “Gastric Treatment/Diagnosis Device and Attachment Device and Method”, U.S. Pat. No. 7,620,454 (Nov. 17, 2009 Dinsmoor et al.) “Gastro-Electric Stimulation for Reducing the Acidity of Gastric Secretions or Reducing the Amounts Thereof”, U.S. Pat. No. 7,643,887 (Jan. 5, 2010 Imran) “Abdominally Implanted Stimulator and Method”, U.S. Pat. No. 7,702,394 (Apr. 20, 2010 Imran) “Responsive Gastric Stimulator”, U.S. Pat. No. 7,738,961 (Jun. 15, 2010 Sharma) “Method and Apparatus for Treatment of the Gastrointestinal Tract”, U.S. Pat. No. 7,742,818 (Jun. 22, 2010 Dinsmoor et al.) “Gastro-Electric Stimulation for Increasing the Acidity of Gastric Secretions or Increasing the Amounts Thereof”, U.S. Pat. No. 7,881,797 (Feb. 1, 2011 Griffin et al.) “Methods and Devices for Gastrointestinal Stimulation”, U.S. Pat. No. 7,941,221 (May 10, 2011 Foley) “Method and Apparatus for Intentional Impairment of Gastric Motility and/or Efficiency by Triggered Electrical Stimulation of the Gastrointestinal . . . ”, U.S. Pat. No. 8,214,049 (Jul. 3, 2012 Brynelsen et al.) “Gastric Stimulation Systems and Methods Utilizing a Transgastric Probe”, and U.S. Pat. No. 8,239,027 (Aug. 7, 2012 Imran) “Responsive Gastric Stimulator”.

Examples of prior art that appear to be best classified in this category also include U.S. patent applications: 20020072780 (Jun. 13, 2002 Foley) “Method and Apparatus for Intentional Impairment of Gastric Motility and/or Efficiency by Triggered Electrical Stimulation of the Gastrointestinal Tract . . . ”, 20030009202 (Jan. 9, 2003 Levine) “Internal Monitoring System with Detection of Food Intake”, 20040059393 (Mar. 25, 2004 Policker et al.) “Regulation of Eating Habits”, 20040088023 (May 6, 2004 Imran et al.) “Gastric Treatment and Diagnosis Device and Method”, 20040162595 (Aug. 19, 2004 Foley) “Method and Apparatus for Intentional Impairment of Gastric Motility and/or Efficiency by Triggered Electrical Stimulation of the Gastrointestinal Tract . . . ”, 20050065571 (Mar. 24, 2005 Imran) “Responsive Gastric Stimulator”, 20050090873 (Apr. 28, 2005 Imran) “Gastrointestinal Stimulation Device”, 20060079944 (Apr. 13, 2006 Imran) “Device and Method for Treating Obesity”, 20060089699 (Apr. 27, 2006 Imran) “Abdominally Implanted Stimulator and Method”, 20070060812 (Mar. 15, 2007 Harel et al.) “Sensing of Pancreatic Electrical Activity”, 20070162085 (Jul. 12, 2007 Dilorenzo) “Method Apparatus Surgical Technique and Stimulation Parameters for Autonomic Neuromodulation for the Treatment of Obesity”, 20080058887 (Mar. 6, 2008 Griffin et al.) “Methods and Devices for Gastrointestinal Stimulation”, 20080086179 (Apr. 10, 2008 Sharma) “Method and Apparatus for Treatment of the Gastrointestinal Tract”, 20090018605 (Jan. 15, 2009 Imran et al.) “Gastric Treatment/Diagnosis Device and Attachment Device and Method”, 20090018605 (Jan. 15, 2009 Imran et al.) “Gastric Treatment/Diagnosis Device and Attachment Device and Method”, 20090030475 (Jan. 29, 2009 Brynelsen et al.) “Gastric Stimulation Systems and Methods Utilizing a Transgastric Probe”, and 20090149910 (Jun. 11, 2009 Imran et al.) “Gastric Treatment/Diagnosis Device and Attachment Device and Method”.

Examples of prior art that appear to be best classified in this category also include U.S. patent applications: 20090264951 (Oct. 22, 2009 Sharma) “Device and Implantation System for Electrical Stimulation of Biological Systems”, 20100049274 (Feb. 25, 2010 Cholette) “Detection of Feeding Intent for Use in Treatment of Eating Disorders”, 20100049274 (Feb. 25, 2010 Cholette) “Detection of Feeding Intent for Use in Treatment of Eating Disorders”, 20100094374 (Apr. 15, 2010 Imran) “Responsive Gastric Stimulator”, 20100305656 (Dec. 2, 2010 Imran et al.) “Gastric Stimulation Anchor and Method”, 20100324432 (Dec. 23, 2010 Bjorling et al.) “Method and Device to Detect Eating to Control Artificial Gastric Stimulation”, 20110004266 (Jan. 6, 2011 Sharma) “Method and Apparatus for Treatment of the Gastrointestinal Tract”, 20110066207 (Mar. 17, 2011 Imran) “Responsive Gastric Stimulator”, 20110125211 (May 26, 2011 Griffin et al.) “Methods and Devices for Gastrointestinal Stimulation”, 20110251495 (Oct. 13, 2011 Province et al.) “Diagnostic Sensors and/or Treatments for Gastrointestinal Stimulation or Monitoring Devices”, 20110295335 (Dec. 1, 2011 Sharma et al.) “Device and Implantation System for Electrical Stimulation of Biological Systems”, 20110295336 (Dec. 1, 2011 Sharma et al.) “Device and Implantation System for Electrical Stimulation of Biological Systems”, 20110307027 (Dec. 15, 2011 Sharma et al.) “Device and Implantation System for Electrical Stimulation of Biological Systems”, 20110307028 (Dec. 15, 2011 Sharma et al.) “Device and Implantation System for Electrical Stimulation of Biological Systems”, 20120277619 (Nov. 1, 2012 Starkebaum et al.) “Detecting Food Intake Based on Impedance”, and 20120316451 (Dec. 13, 2012 Province et al.) “Event Evaluation Using Heart Rate Variation for Ingestion Monitoring and Therapy”.

38. Electrical Stimulation (with Taste Modification)

Devices in this category are similar to devices in the prior category of general electrical stimulation except that they specifically modify a person's sense of taste. In an example, nerves that innervate a person's taste buds can be stimulated to modify a person's sense of taste and thereby modify their food consumption.

Examples of prior art that appear to be best classified in this category include U.S. patent applications: 20060173508 (Aug. 3, 2006 Stone et al.) “Method and System for Treatment of Eating Disorders by Means of Neuro-Electrical Coded Signals”, 20060206169 (Sep. 14, 2006 Schuler) “Method and System for Modulating Eating Behavior by Means of Neuro-Electrical Coded Signals”, 20060235487 (Oct. 19, 2006 Meyer et al.) “Method and System for Treatment of Eating Disorders by Means of Neuro-Electrical Coded Signals”, 20110276112 (Nov. 10, 2011 Simon et al.) “Devices and Methods for Non-Invasive Capacitive Electrical Stimulation and Their Use for Vagus Nerve Stimulation on the Neck of a Patient”, 20120029591 (Feb. 2, 2012 Simon et al.) “Devices and Methods for Non-Invasive Capacitive Electrical Stimulation and Their Use for Vagus Nerve Stimulation on the Neck of a Patient”, 20120029601 (Feb. 2, 2012 Simon et al.) “Devices and Methods for Non-Invasive Capacitive Electrical Stimulation and Their Use for Vagus Nerve Stimulation on the Neck of a Patient”, 20120277814 (Nov. 1, 2012 Schuler) “Method and System for Modulating Eating Behavior by Means of Neuro-Electrical Coded Signals”, and 20120277837 (Nov. 1, 2012 Schuler) “Method and System for Modulating Eating Behavior by Means of Neuro-Electrical Coded Signals”.

39. Electrical Stimulation (with Drug)

Devices in this category are similar to devices in the prior category of general electrical stimulation except that they also include a drug delivery mechanism. In addition to delivering electromagnetic energy to person's gastrointestinal tract or to a nerve that innervates their gastrointestinal tract, devices in this category can also include an implantable drug pump. In an example, electrical stimulation can be used in conjunction with drug delivery to create combined therapeutic effects.

Examples of prior art that appear to be best classified in this category include: U.S. patents U.S. Pat. No. 5,782,798 (Jul. 21, 1998 Rise) “Techniques for Treating Eating Disorders by Brain Stimulation and Drug Infusion”, U.S. Pat. No. 7,493,171 (Feb. 17, 2009 Whitehurst et al.) “Treatment of Pathologic Craving and Aversion Syndromes and Eating Disorders by Electrical Brain Stimulation and/or Drug Infusion”, U.S. Pat. No. 7,835,796 (Nov. 16, 2010 Maschino et al.) “Weight Loss Method and Device”, U.S. Pat. No. 8,010,204 (Aug. 30, 2011 Knudson et al.) “Nerve Blocking for Treatment of Gastrointestinal Disorders”, U.S. Pat. No. 8,185,206 (May 22, 2012 Starkebaum et al.) “Electrical Stimulation Therapy to Promote Gastric Distention for Obesity Management”, and U.S. Pat. No. 8,295,926 (Oct. 23, 2012 Dobak) “Dynamic Nerve Stimulation in Combination with Other Eating Disorder Treatment Modalities”; and U.S. patent applications 20080021512 (Jan. 24, 2008 Knudson et al.) “Nerve Stimulation and Blocking for Treatment of Gastrointestinal Disorders”, 20080262411 (Oct. 23, 2008 Dobak) “Dynamic Nerve Stimulation in Combination with Other Eating Disorder Treatment Modalities”, 20110282411 (Nov. 17, 2011 Knudson et al.) “Nerve Stimulation and Blocking for Treatment of Gastrointestinal Disorders”, 20110282411 (Nov. 17, 2011 Knudson et al.) “Nerve Stimulation and Blocking for Treatment of Gastrointestinal Disorders”, and 20120277661 (Nov. 1, 2012 Bernard et al.) “Method and Apparatus for Delivery of Therapeutic Agents”.

40. Electrical Stimulation (with Drug and Sensor)

Devices in this category are similar to devices in a prior category of general electrical stimulation except that they also include a drug delivery mechanism and at least one sensor. In an example, electrical stimulation can be used in conjunction with drug delivery to create combined therapeutic effects. Further, the sensor can be used to create a self-adjusting, closed-loop stimulation and/or drug delivery system for modification of food consumption.

Examples of prior art that appear to be best classified in this category include: U.S. patents U.S. Pat. No. 6,950,707 (Sep. 27, 2005 Whitehurst) “Systems and Methods for Treatment of Obesity and Eating Disorders by Electrical Brain Stimulation and/or Drug Infusion”, U.S. Pat. No. 7,076,305 (Jul. 11, 2006 Imran et al.) “Gastric Device and Instrument System and Method”, U.S. Pat. No. 7,483,746 (Jan. 27, 2009 Lee et al.) “Stimulation of the Stomach in Response to Sensed Parameters to Treat Obesity”, U.S. Pat. No. 7,590,452 (Sep. 15, 2009 Imran et al.) “Endoscopic System for Attaching a Device to a Stomach”, and U.S. Pat. No. 8,095,219 (Jan. 10, 2012 Lee et al.) “Stimulation of the Stomach in Response to Sensed Parameters to Treat Obesity”; and U.S. patent applications 20030167024 (Sep. 4, 2003 Imran et al.) “Gastric Device and Instrument System and Method”, 20040243195 (Dec. 2, 2004 Imran et al.) “Endoscopic System for Attaching a Device to a Stomach”, 20060129201 (Jun. 15, 2006 Lee et al.) “Stimulation of the Stomach in Response to Sensed Parameters to Treat Obesity”, and 20090299434 (Dec. 3, 2009 Imran et al.) “Endoscopic System for Attaching a Device to a Stomach”.

### 41. Salivation Stimulation

This category of prior art includes devices and methods for stimulating salivation in a person's mouth. In some respects, this is quite different than devices and methods that are intended to reduce food consumption. Most devices and methods in this category are focused on increasing, not decreasing, food consumption. However, this category is included for completeness because some of these devices are intended to modify the early stages of food digestion within a person's mouth, which can be relevant. In an example, devices in this category can apply electrical stimulation to the mouth to increase salivation. In an example, devices in this category can release a salivation-stimulating substance.

Examples of prior art that appear to be best classified in this category include: U.S. patents U.S. Pat. No. 4,519,400 (May 28, 1985 Brenman et al.) “Method for Stimulating Salivation (Biosonics)”, U.S. Pat. No. 4,637,405 (Jan. 20, 1987 Brenman et al.) “Apparatus for Stimulating Salivation”, U.S. Pat. No. 6,230,052 (May 8, 2001 Wolff et al.) “Device and Method for Stimulating Salivation”, U.S. Pat. No. 7,041,311 (May 9, 2006 Grainger et al.) “Preparation for Saliva Flow”, and U.S. Pat. No. 7,477,947 (Jan. 13, 2009 Pines et al.) “System and Method for Electrical Stimulation of Salivation”; and U.S. patent application 20070077300 (Apr. 5, 2007 Wynn et al.) “Oral Compositions Containing a Salivation Inducing Agent”.

### 42. General Sensor (Glucose)

This category of prior art includes sensors and monitors which detect and analyze glucose levels (such as blood glucose levels). These sensors and monitors can be used for a variety of applications other than modification of food consumption or food absorption. For example, they can be used to determine when a diabetic person needs insulin. Nonetheless, overall, they are sufficiently relevant to be included in this review.

Examples of prior art that appear to be best classified in this category include: U.S. patents U.S. Pat. No. 5,497,772 (Mar. 12, 1996 Schulman et al.) “Glucose Monitoring System”, U.S. Pat. No. 7,727,147 (Jun. 1, 2010 Osorio et al.) “Method and System for Implantable Glucose Monitoring and Control of a Glycemic State of a Subject”, U.S. Pat. No. 7,974,672 (Jul. 5, 2011 Shults et al.) “Device and Method for Determining Analyte Levels”, U.S. Pat. No. 7,988,630 (Aug. 2, 2011 Osorio et al.) “Method and System for Implantable Glucose Monitoring and Control of a Glycemic State of a Subject”, U.S. Pat. No. 8,158,082 (Apr. 17, 2012 Imran) “Micro-Fluidic Device”, U.S. Pat. No. 8,236,242 (Aug. 7, 2012 Drucker et al.) “Blood Glucose Tracking Apparatus and Methods”, U.S. Pat. No. 8,275,438 (Sep. 25, 2012 Simpson et al.) “Analyte Sensor”, U.S. Pat. No. 8,287,453 (Oct. 16, 2012 Li et al.) “Analyte Sensor”, and U.S. Pat. No. 8,298,142 (Oct. 30, 2012 Simpson et al.) “Analyte Sensor”; and U.S. patent applications 20050096637 (May 5, 2005 Heruth) “Sensing Food Intake”, 20120078071 (Mar. 29, 2012 Bohm et al.) “Advanced Continuous Analyte Monitoring System”, 20120149996 (Jun. 14, 2012 Stivoric et al.) “Method and Apparatus for Providing Derived Glucose Information Utilizing Physiological and/or Contextual Parameters”, and 20120201725 (Aug. 9, 2012 Imran) “Micro-Fluidic Device”.

### 43. General Sensor (Electromagnetic)

This category of prior art includes sensors and monitors which detect selected patterns of electromagnetic energy that are emitted from a member of a person's body. Such sensors and monitors can be used for a variety of applications other than modification of food consumption or food absorption. Nonetheless, overall, they are sufficiently relevant to be included in this review.

Examples of prior art that appear to be best classified in this category include: U.S. patents U.S. Pat. No. 5,795,304 (Aug. 18, 1998 Sun et al.) “System and Method for Analyzing Electrogastrophic Signal”, U.S. Pat. No. 6,285,897 (Sep. 4, 2001 Kilcoyne et al.) “Remote Physiological Monitoring System”, U.S. Pat. No. 8,192,350 (Jun. 5, 2012 Ortiz et al.) “Methods and Devices for Measuring Impedance in a Gastric Restriction System”, U.S. Pat. No. 8,265,758 (Sep. 11, 2012 Policker et al.) “Wireless Leads for Gastrointestinal Tract Applications”, and U.S. Pat. No. 8,328,420 (Dec. 11, 2012 Abreu) “Apparatus and Method for Measuring Biologic Parameters”; and U.S. patent applications 20080262557 (Oct. 23, 2008 Brown) “Obesity Management System”, 20090281449 (Nov. 12, 2009 Thrower et al.) “Optimization of Thresholds for Eating Detection”, 20100305468 (Dec. 2, 2010 Policker et al.) “Analysis and Regulation of Food Intake”, and 20120316459 (Dec. 13, 2012 Abreu) “Apparatus and Method for Measuring Biologic Parameters”.

### 44. General Sensor (Chemical)

This category of prior art includes sensors which can detect specific types of chemicals. Such sensors can be used for a variety of applications other than modification of food consumption or food absorption. Some are not even directed toward biomedical applications. Nonetheless, overall, they are sufficiently relevant to be included in this review.

Examples of prior art that appear to be best classified in this category include: U.S. patents U.S. Pat. No. 6,218,358 (Apr. 17, 2001 Firestein et al.) “Functional Expression of, and Assay for, Functional Cellular Receptors In Vivo”, U.S. Pat. No. 6,387,329 (May 14, 2002 Lewis et al.) “Use of an Array of Polymeric Sensors of Varying Thickness for Detecting Analytes in Fluids”, U.S. Pat. No. 6,610,367 (Aug. 26, 2003 Lewis et al.) “Use of an Array of Polymeric Sensors of Varying Thickness for Detecting Analytes in Fluids”, U.S. Pat. No. 7,122,152 (Oct. 17, 2006 Lewis et al.) “Spatiotemporal and Geometric Optimization of Sensor Arrays for Detecting Analytes Fluids”, U.S. Pat. No. 7,241,880 (Jul. 10, 2007 Adler et al.) “T1R Taste Receptors and Genes Encoding Same”, U.S. Pat. No. 7,595,023 (Sep. 29, 2009 Lewis et al.) “Spatiotemporal and Geometric Optimization of Sensor Arrays for Detecting Analytes in Fluids”, U.S. Pat. No. 7,651,868 (Jan. 26, 2010 Mcdevitt et al.) “Method and System for the Analysis of Saliva using a Sensor Array”, U.S. Pat. No. 8,067,185 (Nov. 29, 2011 Zoller et al.) “Methods of Quantifying Taste of Compounds for Food or Beverages”, U.S. Pat. No. 8,314,224 (Nov. 20, 2012 Adler et al.) “T1R Taste Receptors and Genes Encoding Same”, and U.S. Pat. No. 8,334,367 (Dec. 18, 2012 Adler) “T2R Taste Receptors and Genes Encoding Same”; and U.S. patent applications 20090261987 (Oct. 22, 2009 Sun) “Sensor Instrument System Including Method for Detecting Analytes in Fluids”, and 20120015432 (Jan. 19, 2012 Adler) “Isolated Bitter Taste Receptor Polypeptides”.

### 45. General Sensor (Microwave)

This category of prior art includes sensors which can detect selected patterns of microwave energy. Such sensors can be used for a variety of applications other than modification of food consumption or food absorption. Nonetheless, overall, they are sufficiently relevant to be included in this review. Examples of prior art that appear to be best classified in this category include U.S. patent applications 20120053426 (Mar. 1, 2012 Webster et al.) “System and Method for Measuring Calorie Content of a Food Sample” and 20130027060 (Jan. 31, 2013 Tralshawala et al.) “Systems and Methods for Non-Destructively Measuring Calorie Contents of Food Items”.

### 46. Sensor (Intraoral)

This category of prior art includes sensors and monitors which are specifically attached or implanted within a person's oral cavity. Examples of prior art that appear to be best classified in this category include: U.S. Pat. No. 8,233,954 (Jul. 31, 2012 Kling et al.) “Mucosal Sensor for the Assessment of Tissue and Blood Constituents and Technique for Using the Same”; and U.S. patent applications 20050263160 (Dec. 1, 2005 Utley et al.) “Intraoral Aversion Devices and Methods”, 20060020298 (Jan. 26, 2006 Camilleri et al.) “Systems and Methods for Curbing Appetite”, 20070106138 (May 10, 2007 Beiski et al.) “Intraoral Apparatus for Non-Invasive Blood and Saliva Monitoring & Sensing”, and 20100209897 (Aug. 19, 2010 Utley et al.) “Intraoral Behavior Monitoring and Aversion Devices and Methods”.

### 47. Sensor (General)

This category of prior art includes general sensors which can be used for a variety of applications other than modification of food consumption or food absorption. Nonetheless, overall, they are sufficiently relevant to merit inclusion in this review.

Examples of prior art that appear to be best classified in this category include: U.S. patents U.S. Pat. No. 4,823,808 (Apr. 25, 1989 Clegg et al.) “Method for Control of Obesity Overweight and Eating Disorders”, U.S. Pat. No. 5,301,679 (Apr. 12, 1994 Taylor) “Method and System for Analysis of Body Sounds”, U.S. Pat. No. 6,365,128 (Apr. 2, 2002 Bennett-Guerrero et al.) “Monitoring Gastrointestinal Function to Guide Care of High Risk Patients”, and U.S. Pat. No. 7,832,407 (Nov. 16, 2010 Gertner) “Obesity Treatment Systems”; and U.S. patent applications 20060089571 (Apr. 27, 2006 Gertner) “Obesity Treatment Systems”, 20090118797 (May 7, 2009 Kliger et al.) “Monitoring, Analysis, and Regulation of Eating Habits”, 20100160745 (Jun. 24, 2010 Hills et al.) “Detection of Food or Drink Consumption in Order to Control Therapy or Provide Diagnostics”, 20120116182 (May 10, 2012 Wong et al.) “Feedback Systems and Methods to Enhance Obstructive and Other Obesity Treatments, Optionally Using Multiple Sensors”, and 20120232361 (Sep. 13, 2012 Birk) “Bariatric Instrument or Accessory with Sensors”.

### 48. Blood Analysis and Monitoring

Prior art in this category includes devices and methods that analyze the flow and/or composition of a person's blood. In an example, a sensor can infer whether a person is consuming food by monitoring blood flow through tissue that is related to food consumption and digestion.

Examples of prior art that appear to be best classified in this category include: U.S. patents U.S. Pat. No. 5,398,688 (Mar. 21, 1995 Laniado) “Method, System and Instrument for Monitoring Food Intake”, U.S. Pat. No. 6,893,406 (May 17, 2005 Takeuchi et al.) “Mastication Monitoring Device”, and U.S. Pat. No. 7,006,871 (Feb. 28, 2006 Darvish et al.) “Blood Glucose Level Control”; and U.S. patent applications 20040073142 (Apr. 15, 2004 Takeuchi et al.) “Mastication Monitoring Device”, and 20110218407 (Sep. 8, 2011 Haberman et al.) “Method and Apparatus to Monitor, Analyze and Optimize Physiological State of Nutrition”.

### 49. General Energy Balance Feedback

This category of prior art includes a wide variety of relatively-general systems, devices, and methods that are intended to provide a person with support and feedback concerning their energy balance and weight management. In various examples, systems, devices, and methods in this category can involve: general feedback and behavior modification concerning diet and exercise patterns; broadly-defined use of general types of sensors for energy balance and weight management; interactive communication between people and healthcare providers, or between people and social support networks; internet websites that provide online support for energy balance and weight management; and general meal planning systems and methods. Much of the prior art in this category can be very useful, but is very general compared to the specificity of this present invention. Nonetheless, this general category is included in this review in order to be thorough.

Examples of prior art that appear to be best classified in this category include: U.S. patents U.S. Pat. No. 4,951,197 (Aug. 21, 1990 Mellinger) “Weight Loss Management System”, U.S. Pat. No. 5,720,771 (Feb. 24, 1998 Snell) “Method and Apparatus for Monitoring Physiological Data from an Implantable Medical Device”, U.S. Pat. No. 6,154,676 (Nov. 28, 2000 Levine) “Internal Monitoring and Behavior Control System (Robert Levine)”, U.S. Pat. No. 6,334,073 (Dec. 25, 2001 Levine) “Internal Monitoring and Behavior Control System”, U.S. Pat. No. 6,735,479 (May 11, 2004 Fabian et al.) “Lifestyle Management System”, U.S. Pat. No. 7,247,023 (Jul. 24, 2007 Peplinski et al.) “System and method for monitoring weight and nutrition (Daniel Peplinski)”, and U.S. Pat. No. 7,882,150 (Feb. 1, 2011 Badyal) “Health Advisor”; and U.S. patent applications 20050113649 (May 26, 2005 Bergantino) “Method and Apparatus for Managing a User's Health”, 20060015016 (Jan. 19, 2006 Thornton) “Caloric Balance Weight Control System and Methods of Making and Using Same”, 20060122468 (Jun. 8, 2006 Tavor) “Nutritional Counseling Method and Server”, 20070021979 (Jan. 25, 2007 Cosentino et al.) “Multiuser Wellness Parameter Monitoring System”, 20080221644 (Sep. 11, 2008 Vallapureddy et al.) “Remote Monitoring and Control of Implantable Devices”, and 20120065706 (Mar. 15, 2012 Vallapureddy et al.) “Remote Monitoring and Control of Implantable Devices”.

### 50. Miscellaneous Energy Balance Related Devices and Methods

Lastly, this category of prior art includes a variety of devices and methods that may be generally relevant to the measurement and modification of food consumption, but which resist neat categorization. Examples of prior art in this miscellaneous category include: altering food perception through the use of special tableware; devices that a person activates to emit a bad smell to reduce their appetite; devices that a person uses to shock their tongue when they have a craving; devices to increase airflow through the nose; methods for identifying olfactory cells; time-restricted food containers to control access to food; and using tongue stimulation as a sensory substitute for vision.

Examples of prior art that appear to be best classified in this category include: U.S. Pat. No. 4,582,492 (Apr. 15, 1986 Etter et al.) “Method for Behavior Modification Using Olfactory Stimuli”, U.S. Pat. No. 5,792,210 (Aug. 11, 1998 Wamubu et al.) “Electrical Tongue Stimulator and Method for Addiction Treatment”, U.S. Pat. No. 6,145,503 (Nov. 14, 2000 Smith) “Olfactory Activator”, U.S. Pat. No. 6,159,145 (Dec. 12, 2000 Satoh) “Appetite Adjusting Tool”, U.S. Pat. No. 7,409,647 (Aug. 5, 2008 Elber et al.) “Control of Interactions Within Virtual Environments”, and U.S. Pat. No. 8,060,220 (Nov. 15, 2011 Liebergesell et al.) “Promotion of Oral Hygiene and Treatment of Gingivitis Other Periodontal Problems and Oral Mal Odor”.

Examples of prior art that appear to be best classified in this category also include U.S. patent applications: 20020049482 (Apr. 25, 2002 Fabian et al.) “Lifestyle Management System”, 20040186528 (Sep. 23, 2004 Ries et al.) “Subcutaneous Implantable Medical Devices with Anti-Microbial Agents for Chronic Release”, 20050146419 (Jul. 7, 2005 Porter) “Programmable Restricted Access Food Storage Container and Behavior Modification Assistant”, 20050240253 (Oct. 27, 2005 Tyler et al.) “Systems and Methods for Altering Vestibular Biology”, 20080141282 (Jun. 12, 2008 Elber et al.) “Control of Interactions Within Virtual Environments”, 20080270947 (Oct. 30, 2008 Elber et al.) “Control of Interactions Within Virtual Environments”, 20090197963 (Aug. 6, 2009 Llewellyn) “Method and Compositions for Suppressing Appetite or Treating Obesity”, 20090312817 (Dec. 17, 2009 Hogle et al.) “Systems and Methods for Altering Brain and Body Functions and for Treating Conditions and Diseases of the Same”, 20100055245 (Mar. 4, 2010 Havekotte et al.) “Modifying Flavor Experience Via Aroma Delivery”, 20100291515 (Nov. 18, 2010 Pinnisi et al.) “Regulating Food and Beverage Intake”, 20110314849 (Dec. 29, 2011 Park et al.) “Storage Container with Sensor Device and Refrigerator Having the Same”, 20120009551 (Jan. 12, 2012 Pinnisi) “Cues to Positively Influence Eating Habits”, 20120036875 (Feb. 16, 2012 Yun et al.) “Storage Container with Sensor Device and Refrigerator Having the Same”, and 20120299723 (Nov. 29, 2012 Hafezi et al.) “Communication System Incorporated in a Container”.

### SUMMARY OF THIS INVENTION

This invention can be embodied in a device and method for selectively reducing a person's excess consumption of one or more selected (unhealthy) nutrients, or foods containing such nutrients, using electrical stimulation. In an example, these nutrients can be selected from the group consisting of: sugars; carbohydrates; fats; cholesterols; and sodium compounds. Excess consumption is consumption that is greater than an allowable amount. When embodied as a device, this invention includes a specific-nutrient-identifying sensor, a gastrointestinal electrical stimulator, and a cumulative-nutrient-consumption regulator.

This invention provides reduced excess consumption of unhealthy nutrients and foods, while still allowing normal consumption of healthy nutrients and foods. This allows a person to lose weight without the deficiencies in essential nutrients that can occur with food-blind bariatric procedures and devices in the prior art that reduce consumption and/or absorption of both healthy and unhealthy food. This novel invention addresses several limitations of the prior art in this field and provides a number of advantages for energy balance, weight management, and proper nutrition over the prior art. Further, its novel features are not anticipated by the prior art.

### INTRODUCTION TO THE FIGURES

FIGS. 1 through 8 show some examples of how this invention can be embodied, but they do not limit the full generalizability of the claims.

FIG. 1 shows an example of how this device can allow healthy food to pass normally through a person's gastrointestinal tract.

FIG. 2 shows an example of how this device can also allow up to an allowable amount of unhealthy food to pass normally through the person's gastrointestinal tract.

FIG. 3 shows an example of how this device can selectively and automatically reduce excessive consumption and/or absorption of unhealthy food by reducing the outflow of food from a person's stomach when the person consumes an excessive amount of unhealthy food.

FIG. 4 shows an example of how this device can selectively and automatically reduce excessive consumption and/or absorption of unhealthy food by increasing food motility through the gastrointestinal tract when the person consumes an excessive amount of unhealthy food.

FIG. 5 shows an example of how this device can selectively and automatically reduce excessive consumption and/or absorption of unhealthy food by creating a sense of fullness and/or satiety when the person consumes an excessive amount of unhealthy food.

FIG. 6 shows an example of how this device can selectively and automatically reduce excessive consumption and/or absorption of unhealthy food by modifying the person's sense of taste when the person consumes an excessive amount of unhealthy food.

FIGS. 7 and 8 show examples of how this device can include an external food-consumption monitor.

### DETAILED DESCRIPTION OF THE FIGURES

FIGS. 1 through 8 show some different examples of how this invention can be embodied in a device and method for selectively reducing excess consumption and/or absorption of selected nutrients or food containing such nutrients, wherein excess consumption is consumption greater than an allowable amount. However, these examples do not limit the full generalizability of the claims.

FIGS. 1 through 3 show one possible embodiment of this invention in the context of a longitudinal cross-sectional view of a person's torso and head that shows the upper portion of their gastrointestinal tract. The upper portion of the gastrointestinal tract that is shown includes the person's oral cavity 101, esophagus 102, esophageal-gastric junction 103, stomach 104, pyloric sphincter 105, and duodenum 106. This embodiment of the invention has three main components: an intraoral specific-nutrient-identifying sensor 107; a gastrointestinal electrical stimulator 109; and a cumulative-nutrient-consumption regulator 110. These three components work together to selectively reduce the person's excess consumption of one or more selected (unhealthy) nutrients and/or food containing such nutrients.

FIGS. 1 through 3 show three different views of the same device at three different times and phases of device operation. FIG. 1 shows how this device responds when the person consumes healthy food. FIG. 2 shows how this device responds when the person consumes up to an allowable amount of unhealthy food (as identified by food containing one or more selected nutrients). FIG. 3 shows how this device responds when the person consumes an excessive amount of unhealthy food (as identified by food containing one or more selected nutrients) greater than an allowable consumption amount.

The three sequential views of the device's operation that are shown in FIGS. 1 through 3 demonstrate how this device can selectively reduce excess consumption of unhealthy food, but still allow normal consumption of healthy food or up to an allowable amount of unhealthy food. In this manner, this invention can help a person to avoid the nutritional deficiencies that can occur with food-blind bariatric procedures and devices in the prior art that blindly reduce consumption and/or absorption of both healthy and unhealthy food.

We now discuss the components of FIG. 1 in more detail. FIG. 1 shows a longitudinal cross-sectional view of the upper portion of a person's gastrointestinal tract, including their oral cavity 101, esophagus 102, esophageal-gastric junction 103, stomach 104, pyloric sphincter 105, and duodenum 106. FIG. 1 also shows one example of how this invention can be embodied in a device for reducing excess consumption of one or more selected nutrients. This embodiment includes intraoral specific-nutrient-identifying sensor 107, gastrointestinal electrical stimulator 109, and cumulative-nutrient-consumption regulator 110.

In the example in FIG. 1, intraoral specific-nutrient-identifying sensor 107 is attached to the palatal vault of the person's oral cavity 101. In other examples, an intraoral specific-nutrient-identifying sensor can be implanted or attached in other locations that are in fluid communication with the person's oral cavity. In other examples, a specific-nutrient-identifying sensor can be implanted elsewhere within a person's body so as to be in fluid or other sensory communication with another gastrointestinal organ and/or with a nerve that innervates such an organ.

In the example in FIG. 1, gastrointestinal electrical stimulator 109 and cumulative-nutrient-consumption regulator 110 are jointly located within housing 108 which is implanted within the person's abdominal cavity. In another example, gastrointestinal electrical stimulator 109 and cumulative-nutrient-consumption regulator 110 can be in different locations and/or housings. In an example, cumulative-nutrient-consumption regulator 110 can be external to the person and in wireless communication with implanted gastrointestinal electrical stimulator 109.

In the example in FIG. 1, gastrointestinal electrical stimulator 109 is in direct electromagnetic communication with the person's stomach 104 via wire 111 and gastric electrode 112. In this example, gastric electrode 112 is attached to the wall of the person's stomach 104. In other examples, a gastrointestinal electrical stimulator can deliver electricity to a different gastrointestinal organ and/or to a nerve that innervates such an organ.

In an example, there can be wireless communication between gastrointestinal electrical stimulator 109 and other components of this invention that are in different locations. In an example, components sharing wired or wireless communication can include: a specific-nutrient-identifying sensor; a gastrointestinal electrical stimulator; and a cumulative-nutrient-consumption regulator.

In various examples, gastrointestinal electrical stimulator 109 can comprise sub-components including: a microprocessor or Central Processing Unit (CPU); a memory; a wireless communications member for receiving and transmitting wireless data; a power source; and an electrical-energy-delivering component. There are gastrointestinal electrical stimulators in the prior art which can be understood by someone of ordinary skill in the art and the specific internal configuration of such a stimulator is not key to this invention, so the specific internal configuration of such a stimulator is not specified here. It is sufficient to specify that the gastrointestinal electrical stimulator: be configured to be implanted within a person's body; and be able to delivery electricity to a gastrointestinal organ and/or to a nerve innervating such an organ so as to reduce a person's consumption and/or absorption of food. As we will see in subsequent figures, delivery of electricity by gastrointestinal electrical stimulator 109 in this example reduces the amount of food that the person consumes by decreasing the speed at which food moves through their stomach.

We now discuss the operation of this embodiment of this invention in more detail. We discuss how this embodiment responds differently to the person's consumption of different types of nutrients and foods containing those nutrients. In this example, intraoral specific-nutrient-identifying sensor 107 automatically analyzes the chemical composition of saliva in the person's oral cavity in order to selectively identify the person's consumption of at least one selected nutrient which is unhealthy when consumed in excess. In various examples, one or more nutrients identified as being unhealthy when consumed in excess can be selected from the group consisting of: a specific sugar, a specific carbohydrate, a specific fat, a specific cholesterol, a specific sodium compound, a category of sugars, a category of carbohydrates, a category of fats, a category of cholesterols, a category of sodium compounds, sugars in general, carbohydrates in general, fats in general, cholesterols in general, and sodium compounds in general.

In an example, specific-nutrient-identifying sensor 107 can analyze the chemical composition of saliva within the person's oral cavity in order to identify consumption of selected nutrients and/or foods containing these nutrients. In an example, specific-nutrient-identifying sensor 107 identifies the chemical composition of micro-samples of saliva in the person's oral cavity. In an example, specific-nutrient-identifying sensor 107 can automatically extract and analyze micro-samples of saliva from a person's oral cavity in order to automatically and selectively detect when the person is digesting food that is high in (simple) sugars or (saturated) fats.

In various examples, specific-nutrient-identifying sensor 107 can be selected from the group consisting of: biological sensor, enzyme-based sensor, protein-based sensor, and/or reagent-based sensor; chemical sensor, biochemical sensor, chemoreceptor, osmolality sensor, and/or pH level sensor; electromagnetic sensor, electrical impedance sensor; glucose sensor, fat sensor, cholesterol sensor, amino acid sensor, and/or micronutrient sensor; Micro Electrical Mechanical System (MEMS) sensor, microfluidic sensor, laboratory-on-a-chip, medichip, and/or membrane-based sensor; optical sensor, optoelectronic sensor, infrared sensor, spectroscopy sensor, and/or chromatography sensor; and sound sensor, acoustic energy sensor, microphone, chewing sensor, swallow detector, and/or ultrasonic sensor.

In this example, intraoral specific-nutrient-identifying sensor 107 is in wireless communication with cumulative-nutrient-consumption regulator 110. In this example, intraoral specific-nutrient-identifying sensor 107 informs cumulative-nutrient-consumption regulator 110 when the person is consuming a selected nutrient (and/or a food containing this selected nutrient). Cumulative-nutrient-consumption regulator 110 keeps track of the cumulative amount of consumption of this selected nutrient and triggers gastrointestinal electrical stimulator 109 to deliver electricity to the person's stomach 104 when the cumulative consumption of this selected nutrient exceeds an allowable amount. In this manner, the gastrointestinal electrical stimulator 109 only delivers electricity to the person's stomach 104 when the person consumes an excessive amount of selected (unhealthy) nutrients and/or food containing such nutrients.

In this example, the amount of consumption of a specific nutrient that is allowed by cumulative-nutrient-consumption regulator 110 is predetermined by a dietary plan created for the person by a health care provider. In this example, this allowable amount is based on the person's cumulative consumption of the selected nutrient per day. In various examples, an allowable amount and/or duration of consumption for a selected nutrient can be based, in whole or in part, on one or more factors selected from the group consisting of: an allowable amount of a selected nutrient per meal; an allowable amount of a selected nutrient per day; an allowable amount of a selected nutrient per week; a rolling average allowable consumption amount based on multiple sequential minutes, hours, or days; a person's recent pattern of eating and nutrient consumption; and a person's speed or pace of eating and nutrient consumption.

In various examples, the amount of consumption of a specific nutrient that is allowed by a cumulative-nutrient-consumption regulator can depend on one or more factors selected from the group consisting of: the type of selected nutrient; the specificity or breadth of the selected nutrient type; the accuracy of the sensor in detecting the selected nutrient; the speed or pace of nutrient consumption; the person's age, gender, and/or weight; changes in the person's weight; the person's diagnosed health conditions; one or more general health status indicators; the magnitude and/or certainty of the effects of past consumption of the selected nutrient on the person's health; achievement of the person's health goals; the person's exercise patterns and/or caloric expenditure; the person's physical location; the time of day; the day of the week; occurrence of a holiday or other occasion involving special meals; input from a social network and/or behavioral support group; input from a virtual health coach; the cost of food; financial payments, constraints, and/or incentives; health insurance copay and/or health insurance premium; the amount and/or duration of the person's consumption of healthy food or nutrients; and a dietary plan created for the person by a health care provider.

In this embodiment of the invention, a specific-nutrient-identifying sensor 107, gastrointestinal electrical stimulator 109, and cumulative-nutrient-consumption regulator 110 work together to selectively reduce excess consumption of one or more selected (unhealthy) nutrients (or food containing such nutrients), but still allow normal consumption of other (healthy) nutrients and food. This invention can discriminate between different types of nutrients and differentially modify their consumption and/or absorption. Further, this ability to selectively modify consumption and/or absorption of selected nutrients can be post-operatively, non-invasively, and reversibly adjusted to change the types and/or quantities of nutrients which are classified as unhealthy versus healthy. This invention can enable a person to lose weight in a healthy manner, without the nutritional deficiencies that are often caused by food-blind bariatric procedures and devices in the prior art that blindly reduce consumption and/or absorption of both healthy and unhealthy nutrients.

In FIG. 1, the consumption (ingestion and digestion) path that is followed by a specific bolus of food 113 as it passes through the person's gastrointestinal tract is represented by a wavy-dotted-line arrow that enters the person's oral cavity 101 and then passes through the person's esophagus 102, esophageal-gastric junction 103, stomach 104, pyloric sphincter 105, and duodenum 105. In FIG. 1, food 113 is healthy food that does not contain one or more selected nutrients which are unhealthy when consumed in excessive quantities.

Since food 113 is healthy food (not containing any selected unhealthy nutrients) this nutrient-discriminating device does not modify the person's consumption of food 113. Normal consumption is indicated by the thickness and spacing of the wavy-dotted-line arrow that represents the consumption (ingestion and digestion) path of food 113. In particular, the wavy-dotted-line arrow representing the path of healthy food 113 is thick (representing normal food quantity) and closely-spaced (representing normal food motility and flow speed). The person is able to consume and absorb healthy nutrients in a normal and unobstructed manner. The person does not suffer from deficiencies of essential healthy nutrients.

In FIG. 1, specific-nutrient-identifying sensor 107 has not identified healthy food 113 as containing a selected nutrient for which excessive consumption should be limited because healthy food 113 does not contain any such nutrients. Accordingly, sensor 107 is not shown as alerting regulator 110 about food 113 in FIG. 1. There is no wireless transmission from sensor 107 to regulator 110 in FIG. 1. Thus, there is no electrical stimulation of the person's stomach 104 by the gastrointestinal electrical stimulator 110 in FIG. 1 and the consumption of food 113 is unmodified in FIG. 1. In this manner, FIG. 1 shows that healthy nutrients (and foods containing those nutrients) are allowed to pass through a person's gastrointestinal tract in an unmodified manner Thus, the person will not suffer from the nutritional deficiencies that can be caused by food-blind consumption and/or absorption reducing procedures and devices in the prior art.

FIG. 2 shows the same embodiment of this device that is shown in FIG. 1, except that now the person is consuming a bolus of food 201 that includes up to an allowable amount of a selected (unhealthy) nutrient. In an example, bolus of food 201 can be classified as “unhealthy” in that it is unhealthy to eat too much of it, but this device allows up to a certain amount of consumption of such “unhealthy” food without triggering electrical stimulation of the person's stomach 104.

In FIG. 2, intraoral specific-nutrient-identifying sensor 107 detects the presence of one or more selected nutrients in food bolus 201. This information is sent from intraoral specific-nutrient-identifying sensor 107 to cumulative-nutrient-consumption regulator 110 via wireless data transmission 202. In FIG. 2, wireless data transmission 202 is symbolically represented by a “lightning bolt” symbol. The origination of wireless data transmission 202 is represented by a lightning bolt symbol extending out from intraoral specific-nutrient-sensor 107. The receipt of wireless data transmission 202 is represented by a lightning bolt symbol extending into housing 108 which contains cumulative-nutrient-consumption regulator 110.

In FIG. 2, the person is consuming food 201 that contains one or more selected (unhealthy) nutrients and the cumulative-nutrient-consumption regulator 110 has been notified of this consumption by wireless data transmission 202 from the intraoral specific-nutrient-identifying sensor 107. However, in FIG. 2, the amount of cumulative consumption of the selected nutrient remains below the allowable amount of consumption for this selected nutrient. This is why the cumulative-nutrient-consumption regulator 110 has not yet triggered the gastrointestinal electrical stimulator 109 to deliver an electrical charge or impulse to the person's stomach 104 in FIG. 2. This is why consumption and/or absorption of the (unhealthy) food is unmodified in FIG. 2. Unmodified consumption of food 201 in FIG. 2 can be confirmed by by reader by observing that the thickness (quantity) and the spacing (motility) of the wavy-dotted-line representing the consumption path of food 201 in FIG. 2 are the same as that of food 113 in FIG. 1.

In an example, allowing unmodified consumption of up to a predefined allowable amount of such food and/or nutrients can reflect research that only excess consumption of such food and/or nutrients is bad for a person's health. Moderate consumption of such food and/or nutrients may not be bad. In an alternative example, consumption of even moderate amounts of such food and/or nutrients can be bad for a person's health, but can still be allowed for the greater good of making the overall operation of the device more acceptable to a person. In an example, allowing moderate consumption of such foods that the person enjoys can be used as an incentive or reward for health-enhancing behavior by the person—such as regular exercise—for an overall positive effect on the person's health.

In various examples, the amount of consumption of a specific nutrient that is allowed by cumulative-nutrient-consumption regulator 110 before it triggers electrical stimulation of the person's stomach can depend on one or more factors selected from the group consisting of: the type of selected nutrient; the specificity or breadth of the selected nutrient type; the accuracy of the sensor in detecting the selected nutrient; the speed or pace of nutrient consumption; the person's age, gender, and/or weight; changes in the person's weight; the person's diagnosed health conditions; one or more general health status indicators; the magnitude and/or certainty of the effects of past consumption of the selected nutrient on the person's health; achievement of the person's health goals; the person's exercise patterns and/or caloric expenditure; the person's physical location; the time of day; the day of the week; occurrence of a holiday or other occasion involving special meals; input from a social network and/or behavioral support group; input from a virtual health coach; the cost of food; financial payments, constraints, and/or incentives; health insurance copay and/or health insurance premium; the amount and/or duration of the person's consumption of healthy food or nutrients; and a dietary plan created for the person by a health care provider.

FIG. 3 shows the same embodiment of this device that was shown in FIGS. 1 and 2, except that now a person's cumulative consumption of a selected nutrient found in food 201 exceeds the allowable amount. In an example, not only is food 201 classified as unhealthy in type (based on the nutrients which it contains), but now the amount being consumed has also become unhealthy in quantity. In FIG. 3, intraoral specific-nutrient-identifying sensor 107 detects the presence of one or more selected nutrients in the person's continued consumption of food 201. This information is sent from intraoral specific-nutrient-identifying sensor 107 to cumulative-nutrient-consumption regulator 110 via wireless data transmission 202.

In FIG. 3, not only is the person consuming food 201 that contains one or more selected (unhealthy) nutrients, but the amount of cumulative consumption of these selected nutrients exceeds the allowable amount of consumption. Accordingly, cumulative-nutrient-consumption regulator 110 triggers the gastrointestinal electrical stimulator 109 to deliver an electrical charge 301 to the person's stomach 104 via wire 111 and gastric electrode 112.

In the example in FIG. 3, electrical charge 301 reduces the person's food consumption because it reduces the outflow of food from the person's stomach 104 through pyloric sphincter 105. In an example, food outflow from the stomach 104 can be slowed because electrical charge 301 increases closure of pyloric sphincter 105. In an example, food outflow from the stomach 104 can be slowed because electrical charge 301 interferes with the stomach's normal motion. In FIG. 3, the reduced outflow of food 201 from the stomach 104 is represented by the expanded food path waves for food 201 as it passes through the stomach 104. Reduced food consumption is represented by the reduced thickness (representing reduced quantity) of the wavy-dotted-line consumption path for food 201 as it passes through the gastrointestinal tract.

In various examples, delivery of electricity to a person's stomach can reduce the person's consumption and/or absorption of food by a mechanism selected from the group consisting of: reducing the amount of food that the person consumes by creating a feeling of fullness and/or satiety; reducing the amount of food that the person consumes by decreasing the speed at which food moves through a portion of the person's gastrointestinal tract; reducing absorption of nutrients from food that the person consumes by increasing the speed at which food moves through a portion of the person's gastrointestinal tract; reducing the amount of food that the person consumes by modifying the person's sense of taste and/or smell; and reducing the amount of food that the person consumes by delivering an unpleasant electrical stimulus to the person's body in response to food consumption.

FIG. 4 shows another embodiment of this invention in a device that selectively reduces a person's excess consumption of one or more selected (unhealthy) nutrients and/or food containing such nutrients. FIG. 4 shows how this embodiment operates when the person consumes an excessive amount of one or more selected nutrients and/or food containing an excessive amount of such nutrients. The device that is shown in FIG. 4 is similar to the device that is shown in FIG. 3 except that the mechanism whereby the device reduces food consumption and/or absorption in FIG. 4 is different than that in FIG. 3. Figures showing operation of this new embodiment with consumption of healthy food or allowable consumption of unhealthy food are not shown because they would be virtually identical to FIGS. 1 and 2.

In the example in FIG. 4, the device reduces absorption of nutrients from food 201 by increasing food motility and flow speed. In FIG. 4, the delivery of an electrical charge 401 from gastrointestinal electrical stimulator 109 to the person's stomach 104 speeds up food motility and flow speed through the person's stomach 104 and duodenum 106. In this example, increased food motility and flow speed (caused by electrical charge 401) decreases: the duration of digestive mixing of food within the stomach; the duration of fluid communication between food and the nutrient-absorbing walls of the duodenum; or both. In this manner, delivery of electrical charge 401 from gastrointestinal electrical stimulator 109 reduces absorption of nutrients from food bolus 201. In the example in FIG. 4, increased food motility and flow speed through the stomach 104 and duodenum 106 is represented by larger spaces in the wavy-dotted-line arrow representing the flow of food bolus 201 through the stomach and duodenum.

In an example, the amount of food 201 that is consumed in FIG. 4 is not reduced, but the amount of nutrients absorbed from food 201 that is consumed is reduced. In an example, increased food motility can be unpleasant for the person. In an example, delivery of electrical charge 401 can wind up reducing the consumption of food 201 in addition to reducing its absorption.

FIG. 5 shows another embodiment of this invention in a device that selectively reduces a person's excess consumption of one or more selected (unhealthy) nutrients and/or food containing such nutrients. FIG. 5 shows how this embodiment operates when the person consumes an excessive amount of one or more selected nutrients and/or food containing an excessive amount of such nutrients. In this respect, the device that is shown in FIG. 5 is similar to the devices that are shown in FIGS. 3 and 4 except that the mechanism whereby the device reduces food consumption and/or absorption in FIG. 5 is different than those in FIGS. 3 and 4. Figures showing operation of this new embodiment with consumption of healthy food or allowable consumption of unhealthy food are not shown because they would be virtually identical to FIGS. 1 and 2.

In the example in FIG. 5, the device reduces consumption of food 201 by creating a sense of fullness and/or satiety for the person. In an example, delivery of an electrical charge 501 to the person's stomach 104 (and/or to a nerve that innervates the person's stomach) mimics the sensation of fullness and/or satiety that the person feels after having eaten a full meal. In this manner, delivery of electrical charge 501 from gastrointestinal electrical stimulator 109 reduces consumption of food 201. In the example in FIG. 5, the reduced food consumption (due to a sense of fullness and/or satiety) is represented by a thinner wavy-dotted-line arrow representing the flow of food 201 through the gastrointestinal tract.

FIG. 6 shows another embodiment of this invention in a device that selectively reduces a person's excess consumption of one or more selected (unhealthy) nutrients and/or food that contains such nutrients. FIG. 6 shows how this embodiment operates when the person consumes an excessive amount of one or more selected nutrients and/or food containing an excessive amount of such nutrients. Figures showing consumption of healthy food or an allowable amount of unhealthy food would not add value to this disclosure and thus are not shown.

The device that is shown in FIG. 6 has two main differences from the devices shown in FIGS. 3 through 5. First, the device in FIG. 6 has a different mechanism for reducing food consumption via electrical stimulation. It modifies a person's sense of taste. Second, the device in FIG. 6 shows the housing for the gastrointestinal electrical stimulator and cumulative-nutrient-consumption regulator in a location that is closer to the person's tongue. In FIG. 6, the housing 601 for gastrointestinal electrical stimulator 602 and cumulative-nutrient-consumption regulator 603 is implanted in the upper right portion of the person's torso so as to be closer to the person's tongue (and/or a nerve that innervates the person's tongue). In an example, housing 601 can be implanted subcutaneously.

With respect to mode of action, the device shown in FIG. 6 reduces a person's consumption of food 201 by modifying the person's sense of taste. In an example, the device delivers an electrical charge 606 (via wire 604 and electrode 605) to the person's tongue (and/or to a nerve that innervates the person's tongue) that reduces or blocks the person's sense of taste. In an example, activation of gastrointestinal electrical stimulator 602 causes the person to have temporary ageusia. Since a person's appetite is heavily influenced by their sense of taste, this causes the person to consume less food. In FIG. 6, reduced food consumption is represented by a thinner wavy-dotted-line arrow indicating the flow of food 201 through the gastrointestinal tract.

In a variation on the device that is shown in FIG. 6, a device can reduce a person's consumption of food 201 by delivery of an electrical charge 606 to the person's tongue (and/or to a nerve that innervates the person's tongue) that creates a virtual taste. In an example, this virtual taste may not be unpleasant by itself, but is not a good match with the food 201 that the person is consuming. In another example, this virtual taste can actually be unpleasant. In an example, this virtual taste can cause this person to consume less food.

FIG. 7 shows another example of how this invention can be embodied in a device that selectively reduces a person's excess consumption of one or more selected nutrients and/or food containing such nutrients. This device includes the three key components of the devices that were shown in FIGS. 1 through 6 (an intraoral specific-nutrient-identifying sensor, a gastrointestinal electrical stimulator, and a cumulative-nutrient-consumption regulator) plus a generic-food-consumption monitor. A generic-food-consumption monitor detects when there is a high probability that the person is consuming food.

In an example, a generic-food-consumption monitor can operate in a more-continuous manner than a specific-nutrient-identifying sensor, but the generic-food-consumption monitor has less ability to detect consumption of specific nutrients than does a specific-nutrient-identifying sensor. In an example, the operation of a specific-nutrient-identifying sensor can be triggered by food consumption as indicated by a generic-food-consumption monitor. In an example, a generic-food-consumption monitor and a specific-nutrient-identifying sensor can operate together to provide greater nutrient discrimination ability, in a more-efficient and less-invasive manner, than either member working alone.

In an example, a generic-food-consumption monitor can work in conjunction with a specific-nutrient-identifying sensor. In an example, a generic-food-consumption monitor can operate in a more-continuous manner to detect general food consumption, a specific-nutrient-identifying sensor can operate in a less-continuous manner to detect consumption of a specific nutrient, and operation of the specific-nutrient-identifying sensor can be triggered, activated, and/or augmented when general food consumption is indicated by the generic-food-consumption monitor. Such cooperative operation of a more-continuous generic-food-consumption monitor and a less-continuous specific-nutrient-identifying sensor can selectively identify consumption of specific nutrients more efficiently, with lower energy requirements, and with less privacy intrusion than is possible with either the monitor or the sensor operating in isolation.

In some respects, the device shown in FIG. 7 is similar to the device shown in FIG. 3, but there are two differences. First, the device in FIG. 7 includes an external generic-food-consumption monitor 702 that is worn around the person's neck on a necklace 701. In this example, generic-food-consumption monitor includes a microphone and continually monitors acoustic energy for chewing and/or swallowing sounds which indicate a high probability that the person is consuming food. Second, sensing by the specific-nutrient-identifying sensor in FIG. 7 is only activated when the generic-food-consumption sensor indicates a high probability that the person is consuming food. This can be more energy efficient and less-intrusive than having specific-nutrient-identifying sensor 107 active all the time.

The overall sequence of operation for the device in FIG. 7 is as follows: The generic-food-consumption monitor 702 operates continuously to detect chewing or swallowing sounds that indicate a high probability that the person is consuming food. When generic-food-consumption monitor 702 detects chewing or swallowing sounds, it sends a wireless signal 703 to activate specific-nutrient-identifying sensor 107.

When activated, the specific-nutrient-identifying sensor 107 starts analyzing the chemical composition of micro-samples of saliva from the person's oral cavity in order to detect consumption of one or more selected (unhealthy) nutrients and/or foods containing such nutrients. When specific-nutrient-identifying sensor 107 detects consumption of these selected nutrients and/or foods containing such nutrients, it sends a wireless signal 202 to cumulative-nutrient-consumption regulator 110.

When cumulative-nutrient-consumption regulator 110 determines that the person has consumed more than an allowable amount of a selected nutrient and/or food containing such a nutrient, then cumulative-nutrient-consumption regulator 110 activates gastrointestinal electrical stimulator 109. When activated, gastrointestinal electrical stimulator 109 delivers an electrical charge 301 to the person's stomach. Electrical charge 301 reduces the flow of food 201 out of the person's stomach, which reduces the amount of food that the person consumes.

In this manner, the device in FIG. 7 can selectively reduce excess consumption of one or more selected (unhealthy) nutrients and/or food containing these nutrients, while allowing normal consumption of other (healthy) nutrients and up to an allowable amount of selected (unhealthy) nutrients. A possible advantage of the embodiment in FIG. 7 over previous embodiments is that the specific-nutrient sensor only needs to operate when there is a high probability that the person is consuming food. This can save energy and/or extend the useful life of the specific-nutrient sensor. A possible disadvantage of the embodiment in FIG. 7 is that it is easier for a person to tamper with the operation of an external monitor (or sensor) than an implanted one.

FIG. 8 shows another example of how this invention can be embodied in a device to selectively reduce excess consumption of one or more selected nutrients and/or food containing such nutrients. FIG. 8 combines the taste-modification mode of action that was introduced in FIG. 6 with the generic-food-consumption monitor that was introduced in FIG. 7.

The overall sequence of operation for the device in FIG. 8 is as follows. Generic-food-consumption monitor 702 operates continuously to detect chewing or swallowing sounds that indicate a high probability that the person is consuming food. When generic-food-consumption monitor 702 detects chewing or swallowing sounds, it sends a wireless signal 703 to activate specific-nutrient-identifying sensor 107.

When activated, the specific-nutrient-identifying sensor 107 starts analyzing the chemical composition of micro-samples of saliva from the person's oral cavity in order to detect consumption of one or more selected (unhealthy) nutrients and/or foods containing such nutrients. When specific-nutrient-identifying sensor 107 detects consumption of these selected nutrients and/or foods containing such nutrients, it sends a wireless signal 202 to cumulative-nutrient-consumption regulator 603.

When cumulative-nutrient-consumption regulator 603 determines that the person has consumed more than an allowable amount of a selected nutrient and/or food containing such a nutrient, then cumulative-nutrient-consumption regulator 603 activates gastrointestinal electrical stimulator 602. When activated, gastrointestinal electrical stimulator 602 delivers an electrical charge 606 to the person's tongue (or a nerve that innervates the person's tongue). Electrical charge 301 modifies the person's sense of taste which reduces the amount of food that the person consumes.

In this manner, the device in FIG. 8 can selectively reduce excess consumption of one or more selected (unhealthy) nutrients and/or food containing these nutrients, while allowing normal consumption of other (healthy) nutrients and up to allowable amounts of selected (unhealthy) nutrients.

FIGS. 1 through 8 show various examples of how this invention can be embodied in a device and method that includes a specific-nutrient-identifying sensor. We now discuss, in greater detail and example variation, how a specific-nutrient-identifying sensor can be configured and can function.

A specific-nutrient-identifying sensor (or specific-nutrient detector or monitor) can selectively detect a person's consumption of a specific nutrient, a specific nutrient type, and/or a food that includes such a specific nutrient. This level of nutrient-specific identification is not provided by generic-food-consumption monitors in the prior art that can only sense when a person is consuming food in general (or perhaps gross differentiation between consumption of solid vs. liquid food), but which cannot selectively identify which specific nutrients a person is consuming. For the purposes of this disclosure, food consumption is defined broadly to include consumption of liquid beverages and gelatinous food, as well as solid food.

In an example, a specific-nutrient-identifying sensor can selectively detect and quantify a person's consumption and/or digestion of one or more selected nutrients, nutrient types, or food containing such nutrients. In an example, a selected nutrient (or nutrient type or food that contains such a nutrient) can be selected by the person whose consumption is being monitored, by a health care provider, by an automated diet program, or by other means. In an example, a specific nutrient can be classified as “unhealthy” because it is unhealthy when it is consumed in excessive quantities. In an example, a specific-nutrient-identifying sensor can be part of a system for selectively reducing excessive consumption and/or absorption of unhealthy nutrients, but still allowing normal consumption and/or absorption of healthy nutrients.

In various examples, one or more of the following nutrients or nutrient types can be identified as being “unhealthy” when consumed in excessive quantities: a specific sugar, a specific carbohydrate, a specific fat, a specific cholesterol, a specific sodium compound, a category of sugars, a category of carbohydrates, a category of fats, a category of cholesterols, a category of sodium compounds, sugars in general, carbohydrates in general, fats in general, cholesterols in general, and sodium compounds in general. In various examples, one or more specific nutrients or specific nutrient types (to be detected by the nutrient-specific sensor) can be selected from the group consisting of: a specific sugar, a specific carbohydrate, a specific fat, a specific cholesterol, a specific sodium compound, a category of sugars, a category of carbohydrates, a category of fats, a category of cholesterols, a category of sodium compounds, sugars in general, carbohydrates in general, fats in general, cholesterols in general, and sodium compounds in general.

In various examples, a specific-nutrient-identifying sensor can selectively detect and quantify a person's consumption of: a specific sugar, a specific carbohydrate, a specific fat, a specific cholesterol, a specific sodium compound, a category of sugars, a category of carbohydrates, a category of fats, a category of cholesterols, a category of sodium compounds, sugars in general, carbohydrates in general, fats in general, cholesterols in general, and/or sodium compounds in general. In various examples, a specific-nutrient-identifying sensor can selectively detect consumption of unhealthy food based on such food having a high concentration, or large amount, of nutrients selected from the group consisting of: sugars, simple sugars, simple carbohydrates, fats, saturated fats, fat cholesterol, and sodium. In various examples, a specific-nutrient-identifying sensor can selectively detect consumption and/or digestion of one or more selected types of foods selected from the group consisting of: fried food, high-cholesterol food, high-fat food, high-sugar food, and high-sodium food.

Many Americans consume highly-processed foods whose primary ingredients include multiple types of sugar. The total amount of sugar is often obscured or hidden, even from those who read ingredients on labels. Sometimes sugar is disguised as “evaporated cane syrup.” Sometimes different types of sugar (“plain sugar,” “brown sugar,” “maltose”, “dextrose,” “evaporated cane syrup,” etc.) are used as different ingredients in a single food item. In such cases, “sugar” does not appear as the main ingredient. However, when one adds up all the different types of sugar in different priority places on the ingredient list, then sugar (in general) really is the main ingredient. These highly-processed conglomerations of sugar (often including corn syrup, fats, and/or caffeine) often have colorful labels with cheery terms like “100% natural” or “high-energy.” However, they are unhealthy when eaten in the quantities to which many Americans have become accustomed. It is no wonder that there is an obesity epidemic in America. In an example, the specific-nutrient-identifying sensor disclosed herein would not be fooled by deceptive labeling on highly-processed conglomerations of sugar, corn syrup, fat, and/or caffeine.

In an example, this invention can be embodied in a device and method that selectively reduces excess consumption and/or absorption of unhealthy food, but still allows normal consumption and absorption of healthy food. The ability of this invention to discriminate between different types of nutrients and to differentially modify their consumption and/or absorption can be post-operatively, non-invasively, and reversibly adjusted and/or programmed to change the types and/or quantities of nutrients which are classified as unhealthy versus healthy. In an example, this invention can encourage a person to consume less unhealthy food and more healthy food. In an example, this invention can enable a person to lose weight in a healthy manner, without the nutritional deficiencies that are often caused by food-blind bariatric procedures and devices in the prior art that blindly reduce absorption of both healthy and unhealthy nutrients.

In an example, a specific-nutrient-identifying sensor can use chemical analysis to identify specific types of nutrients or foods containing those nutrients as these foods are starting to be digested within a person's mouth. In an example, a specific-nutrient-identifying sensor can use infrared spectroscopy to identify the chemical composition of food, saliva, or other material in a person's oral cavity. In an example, a specific-nutrient-identifying sensor can automatically take micro-specimens to chemically analyze the composition of a person's saliva in order to automatically and selectively detect when a person is digesting food that is high in (simple) sugars or (saturated) fats. In an example, a specific-nutrient-identifying sensor can perform intraoral chemical, optical, and/or electromagnetic analysis in order to differentiate between consumption of unhealthy food versus healthy food. In various examples, a specific-nutrient-identifying sensor can detect the amount or concentration of sugars, simple carbohydrates, fats, saturated fats, cholesterol fat, and/or sodium in food, as food is being digested within a person's mouth.

In various examples, one or more specific-nutrient-identifying sensors can be selected from the group consisting of: biological sensor, enzyme-based sensor, protein-based sensor, and/or reagent-based sensor; camera, imaging sensor, and/or pattern recognition sensor; chemical sensor, biochemical sensor, chemoreceptor, osmolality sensor, and/or pH level sensor; electromagnetic sensor, EGG sensor, EMG sensor, electrical impedance sensor, interferometer, muscle activity sensor, and/or neural impulse sensor; glucose sensor, fat sensor, cholesterol sensor, amino acid sensor, and/or micronutrient sensor; Micro Electrical Mechanical System (MEMS) sensor, microfluidic sensor, laboratory-on-a-chip, medichip, and/or membrane-based sensor; motion sensor, movement sensor, accelerometer, flow sensor, strain gauge, electrogoniometer, and/or peristalsis sensor; optical sensor, optoelectronic sensor, infrared sensor, spectroscopy sensor, and/or chromatography sensor; pressure sensor; sound sensor, acoustic energy sensor, microphone, chewing sensor, swallow detector, and/or ultrasonic sensor; taste sensor, olfactory sensor, and/or electronic nose; and temperature sensor, and/or thermistor.

In an example, a specific-nutrient-identifying sensor can be configured to be implanted within, or attached to, a person's oral cavity or implanted in another place within the person's body that enables sensory communication with the person's gastrointestinal tract. There are advantages to having a specific-nutrient-identifying sensor be implanted somewhere within a person's body. An implanted sensor can be more consistent and automatic its food sensing or monitoring function than an external sensor. Also, an implanted sensor is less prone to compliance problems or circumvention on the part of the person whose food consumption is being monitored than is an external sensor. An implanted sensor can be less dependent on voluntary action by the person being monitored than is an external sensor.

As another advantage of an implanted specific-nutrient-identifying sensor, such a sensor can provide types of information concerning food consumption that cannot be obtained with an external sensor. For example, an implanted sensor can analyze food via direct fluid communication with food (such as direct fluid communication with saliva or chyme) which is generally not available with external devices such as mobile phones and cameras, wearable motion sensors, and wearable sound sensors. There are specialized nutrient-analyzing utensils, but they do not work if the person eats food with a generic utensil. In various examples, an implanted specific-nutrient-identifying sensor can be implanted or attached within a person's body by one or more means selected from the group consisting of: adhesive, glue, clamp, wire, clip, pin, snap, elastic member, suture, staple, tissue pouch, fibrotic tissue, screw, and tissue anchor.

In an example, an implanted specific-nutrient-identifying sensor can be an intraoral specific-nutrient-identifying sensor. In an example, an intraoral specific-nutrient-identifying sensor can be implanted so as to be in fluid, chemical, electromagnetic, neural, optical, acoustic, mechanical, and/or other sensory communication with a person's oral cavity and/or nasal cavity. In an example, an intraoral specific-nutrient-identifying sensor can take periodic or specially-activated micro-specimens of saliva within a person's mouth to determine the chemical composition of saliva. In an example, an intraoral specific-nutrient-identifying sensor can perform periodic or specially-activated optical, ultrasonic, or electromagnetic scans of material in a person's mouth in order to determine its chemical composition. In an example, a specific-nutrient-identifying sensor may only analyze specimens or perform scans when a generic-food-consumption monitor indicates that a person is consuming food. Relying on a generic-food-consumption monitor to trigger a specific-nutrient-identifying sensor can be more resource-efficient than having a specific-nutrient-identifying sensor analyze specimens or perform scans in a continuous or constant-periodic manner.

In various examples, an implanted specific-nutrient-identifying sensor can detect consumption of a specific nutrient by one or more mechanisms selected from the group consisting of: biochemical specimen analysis, continuous chemical monitoring, chromatography, electrochemical specimen analysis, electromagnetic monitoring, electroosmotic sampling, electrophoresis, electroporation, enzymatic specimen analysis, infrared spectroscopy, MEMS-based analysis, microfluidic analysis, neurological monitoring, automated periodic sampling; piezoelectric analysis, and ultrasonic monitoring.

In an example, an intraoral specific-nutrient-identifying sensor can be implanted (or attached) within a person's oral cavity so as to be in fluid and/or gaseous communication with food, saliva, and/or other material within a person's oral cavity. In an example, an intraoral specific-nutrient-identifying sensor may not be physically located within a person's oral cavity, but may be in fluid and/or gaseous communication with the oral cavity by means of an artificial lumen. In an example, the specific-nutrient-identifying sensor may withdraw micro-specimens of intraoral fluid or gas from the oral cavity through this artificial lumen.

In an example, an intraoral specific-nutrient-identifying sensor can be implanted within a person's body so as to be in electromagnetic and/or electrochemical communication with nerves that innervate a person's oral cavity and/or nasal cavity. In an example, a specific-nutrient-identifying sensor may be in electromagnetic and/or electrochemical communication with a person's taste buds. In an example, a specific-nutrient-identifying sensor may be in electromagnetic communication with a person's geniculate ganglion and/or petrosal ganglion.

In various examples, an intraoral specific-nutrient-identifying sensor can be configured to be implanted within a person's oral and/or nasal cavity in a location selected from the group consisting of: attached to a person's palatal vault and/or upper roof of their mouth; attached to a person's teeth, such as the buccal spaces, with an adhesive, band, wire, or other fastening mechanism; attached or implanted within a person's mouth using a bio-adhesive, bone screw, or other fastening mechanism; implanted within a person's teeth or within a dental or orthodontic prosthesis such as a retainer, denture, bridge, cap, or crown; implanted into a person's soft palatal tissue; implanted into a person's tongue; implanted within a person's body so as to be in electromagnetic communication with nerves that innervate a person's tongue; implanted within a person's body so as to be in electromagnetic communication with the geniculate ganglion and/or petrosal ganglion; implanted within the sublingual space of a person's oral cavity; implanted within a person's body so as to be in fluid and/or gaseous communication with the oral cavity via an artificial lumen; and implanted within a person's nasal cavity so as to be in fluid and/or gaseous communication with a person's oral cavity.

In various examples, an intraoral specific-nutrient-identifying sensor can be configured to be located in one of the following locations within a person's body: implanted within a person's mouth or oral cavity; attached or implanted with a person's soft palate; attached or implanted within a person's teeth; attached to or implanted within a person's tongue. In various examples, a specific-nutrient-identifying sensor can be configured to be attached to, or implanted within, a person's hard palate, palatal vault and/or upper mouth roof, teeth, tongue, or soft palate. In an example, an intraoral specific-nutrient-identifying sensor can be attached to a person's palatal vault with dental adhesive. In an example, an intraoral specific-nutrient-identifying sensor can be attached to a person's palatal vault with bone screws. In an example, an intraoral specific-nutrient-identifying sensor can be flexible, have a smooth lubricious exterior, and be shaped to match the contour of underlying tissue in order to minimize tissue irritation within a person's mouth. In an example, such a specific-nutrient-identifying sensor can be configured so as to minimize interference with a person's eating, talking, and breathing functions.

In an example, a specific-nutrient-identifying sensor can be configured to be attached to, implanted within, or attached underneath a person's tongue. In an example, a specific-nutrient-identifying sensor can be inserted into a person's tongue. In an example, a specific-nutrient-identifying sensor can be attached or implanted sublingually. In an example, a specific-nutrient-identifying sensor can be configured to be attached to, or inserted into, the soft palate tissues at the rear of a person's oral cavity. In an example, an intraoral sensor can be sealed to protect its internal components from intraoral fluids or gases, except for a controllable opening, lumen, membrane, and/or filter that is used to collect specimens of food, saliva, and/or other oral fluids within a specimen reservoir for chemical analysis. In an example, a microfluidic or MEMS component can be used for specimen collection. In an example, a specific-nutrient-identifying sensor can analyze the chemical composition of food, saliva, other intraoral fluid, and/or intraoral gas cavity in order to detect and quantify consumption of one or more selected nutrients.

There can be advantages to having a specific-nutrient-identifying sensor be an intraoral sensor that is implanted within, attached to, and/or in fluid communication with a person's oral cavity and/or nasal cavity. An intraoral specific-nutrient-identifying sensor that is located in this “upstream” location within a person's gastrointestinal tract can detect consumption and/or digestion earlier than a “downstream” sensor (such as an intragastric sensor). A sensor in a person's mouth can detect food at the point of initial consumption, just as it is starting to be digested. In this manner, an intraoral sensor can provide advance notice that a bolus of food will be coming down the esophagus to enter the stomach. Such advance notice can provide more lead time (no pun intended) for activation of a gastrointestinal electrical stimulator in order to selectively modify the consumption, digestion, and/or absorption of unhealthy food that is coming down the gastrointestinal tract.

In an example, an intraoral specific-nutrient-identifying sensor can identify the types of nutrients being consumed as food begins to be digested within a person's mouth. In an example, a specific-nutrient-identifying sensor that is in fluid communication with a person's mouth can identify nutrients and/or food containing those nutrients as being unhealthy using one or more methods selected from the group consisting of: chemical analysis of food as it begins to be digested within a person's mouth; olfactory analysis of food as it beings to be digested within a person's mouth; image analysis of images of food as it approaches the person's mouth; sonic analysis of chewing or swallowing as food is consumed; and analysis of signals from nerves that innervate a person's taste buds and/or olfactory receptors. In an example, an intraoral specific-nutrient-identifying sensor can be implanted or attached so as to measure the movements, motion, and/or exerted pressure of a person's jaw, teeth, lips, tongue, or other portions of a person's mouth.

Although there can be advantages to having a specific-nutrient-identifying sensor be implanted within a person's oral cavity, in alternative embodiments of this invention a nutrient-specific sensor can be implanted elsewhere within a person's body. More generally, a specific-nutrient-identifying sensor can be implanted anywhere within a person's body that is in fluid, gaseous, chemical, electromagnetic, neural, optical, acoustic, mechanical or other sensory communication with a person's gastrointestinal organs and/or the nerves that innervate those organs. In various examples, a specific-nutrient-identifying sensor can be implanted within (attached to or otherwise in sensory communication with) a person's oral cavity, nasal cavity, esophagus, stomach, duodenum, other portions of a person's intestine, and/or nerves that innervate these members.

In various examples, a specific-nutrient-identifying sensor can be implanted so as to be in fluid, gaseous, chemical, electromagnetic, neural, optical, acoustic, mechanical or other sensory communication with a person's esophagus, including the Lower Esophageal Sphincter (LES) and/or Upper Esophageal Sphincter (UES). In various examples, a specific-nutrient-identifying sensor can be implanted so as to be in fluid, gaseous, chemical, electromagnetic, neural, optical, acoustic, mechanical or other sensory communication with a person's stomach, including the cardia, pyloric sphincter, fundus, lesser curvature, and/or greater curvature.

In various examples, a specific-nutrient-identifying sensor can be implanted so as to be in fluid, gaseous, chemical, electromagnetic, neural, optical, acoustic, mechanical or other sensory communication with a person's duodenum and/or other portions of the person's intestine. In various examples, a specific-nutrient-identifying sensor can be implanted so as to be in fluid, gaseous, chemical, electromagnetic, neural, optical, acoustic, mechanical or other sensory communication with a person's pancreas or liver. In various examples, a specific-nutrient-identifying sensor can be implanted so as to be in electromagnetic, electrochemical, neural, or other sensory communication with a person's vagus nerve, splanchnic nerve, geniculate ganglion, petrosal ganglion, and/or other portions of the person's nervous system that innervate the person's gastrointestinal organs.

In an example, a specific-nutrient-identifying sensor can comprise one or more components selected from the group consisting of: an electronic or optical microprocessor or Central Processing Unit (CPU); a power source and/or energy transducer; a diagnostic energy emitter and/or receiver; a specimen collection mechanism; a specimen-holding reservoir; a reagent-holding reservoir; and a wireless communications component that can wirelessly transmit and receive data. In an example, there can be wireless communication between a specific-nutrient-identifying sensor and other components of this device if these components are in separate locations. In an example, components sharing wireless communication can include: a specific-nutrient-identifying sensor; a gastrointestinal electrical stimulator; a cumulative-nutrient-consumption regulator; and a generic-food-consumption monitor.

In various examples, a specific-nutrient-identifying sensor can be powered by an internal power source such as a rechargeable battery, microchip, or capacitor. In an example, a power source can be recharged from an external source by electromagnetic induction. In various examples, a specific-nutrient-identifying sensor can be powered an external power source. In various examples, a specific-nutrient-identifying sensor can be powered by a combination of internal and external power sources. In various examples, a specific-nutrient-identifying sensor (or a power source within it) can transduce kinetic, thermal, or biochemical energy from within a person's body in order to power the sensor.

In various examples, a specific-nutrient-identifying sensor can be powered from one or more energy sources selected from the group consisting of: a battery, an energy-storing chip, energy harvested or transduced from a bioelectrical cell, energy harvested or transduced from an electromagnetic field, energy harvested or transduced from an implanted biological source, energy harvested or transduced from blood flow or other internal fluid flow, energy harvested or transduced from body kinetic energy, energy harvested or transduced from glucose metabolism, energy harvested or transduced from muscle activity, energy harvested or transduced from organ motion, and energy harvested or transduced from thermal energy.

Although there are advantages to having a specific-nutrient-identifying sensor be implanted within a person's body (such as within the person's oral cavity) a specific-nutrient-identifying sensor can be external to a person's body. In an example, a person can wear an external specific-nutrient-identifying sensor on their body. In an example, an external specific-nutrient-identifying sensor can be incorporated into a mobile electronic device. One advantage of having a specific-nutrient-identifying sensor be external to a person's body is that an external sensor can be less invasive and less-costly than an implanted sensor. As another advantage, an external sensor can detect food-consumption sooner than an implanted one. In an example, an external sensor can detect probable food consumption as a person reaches for food, brings it up to their mouth, and/or inserts it into their mouth. As another potential advantage of an external sensor, some types of food identification are easier when performed before food is inserted into a person's mouth. For example, image-based analysis to determine food type is generally easier when food is on a plate (or in a labeled container) than when it is being chewed within a person's mouth.

In various examples, this invention can include an external specific-nutrient-identifying sensor that sends information in a wireless manner to an internal cumulative-nutrient-consumption regulator. In an example, an external specific-nutrient-identifying sensor can be wearable, portable, and/or mobile. In an example, an external specific-nutrient-identifying sensor can be incorporated into a mobile electronic device, such as a cell phone, mobile phone, or electronic tablet that is carried by a person. In an example, an external specific-nutrient-identifying sensor, or a mobile device of which this sensor is an application or component, can communicate with the internet and/or other mobile devices. In an example, an external sensor, or piece of electronically-functional jewelry of which this sensor is a part, can communicate with the internet and/or other people via other electronic communication means.

In an example, a person can wear an external specific-nutrient-identifying sensor. In various examples, a person can wear an external specific-nutrient-identifying sensor on their wrist, hand, finger, arm, torso, neck, head, and/or ear. In an example, a person can wear an external specific-nutrient-identifying sensor on their clothing. In an example, an external nutrient-specific sensor can be incorporated into a specific article of clothing. In an example, an external nutrient-specific sensor can act as a piece of jewelry or be incorporated into a piece of electronically-functional jewelry. In an example, an external nutrient-specific sensor can be incorporated into a necklace than monitors a person's behavior for eating sounds and/or takes pictures of food. In an example, an external nutrient-specific sensor can be incorporated into a wrist-watch-like member that monitors a person's behavior for eating sounds and/or takes pictures of food. In various examples, an external specific-nutrient-identifying sensor can be incorporated into one or more of the following wearable members: wrist watch, bluetooth device, bracelet, arm band, button, earring, eyeglasses, finger ring, headphones, hearing aid, necklace, nose ring, and pendant.

FIGS. 1 through 8 show various examples of how this invention can be embodied in a device and method that includes a gastrointestinal electrical stimulator. We now discuss, in greater detail and example variation, how a gastrointestinal electrical stimulator can be configured and can function.

This invention can be embodied in a device that includes a gastrointestinal electrical stimulator that delivers electricity to a gastrointestinal organ and/or to a nerve that innervates such an organ. In an example, a gastrointestinal electrical stimulator can deliver an electrical charge, pulse, signal, or field to a gastrointestinal organ and/or to a nerve that innervates such an organ. In various examples, a gastrointestinal electrical stimulator can be configured to be implanted within a person's body so as to be in electromagnetic communication with a person's oral cavity, nasal cavity, esophagus, gastro-esophageal junction, stomach, duodenum, other portions of the intestine, and/or nerves that innervate these members. In various examples, a gastrointestinal electrical stimulator can be attached to, or implanted within, a person's body by one or more means selected from the group consisting of: sutures, staples, adhesive, glue, clamps, clips, pins, snaps, an elastic member, a tissue pouch, fibrotic tissue, screws, and tissue anchors.

In an example, an implanted gastrointestinal electrical stimulator can be in electromagnetic communication with a person's esophagus, including their Lower Esophageal Sphincter (LES) and/or Upper Esophageal Sphincter (UES). In an example, a gastrointestinal electrical stimulator can be in electromagnetic communication with a person's stomach, including the gastric cardia, pyloric sphincter, fundus, lesser curvature, and/or greater curvature. In an example, a gastrointestinal electrical stimulator can be in electromagnetic communication with a person's duodenum and/or other portions of their intestine. In an example, a gastrointestinal electrical stimulator can be in electromagnetic communication with a person's pancreas or liver.

In various examples, a gastrointestinal electrical stimulator can be configured to be attached to, or implanted within, a person's mouth, tongue, esophagus, stomach, duodenum, jejunum, ileum, caecum, or colon. In various examples, a gastrointestinal electrical stimulator can be configured to be implanted within a person's abdominal cavity with a means of electromagnetic communication with a person's esophagus, stomach, duodenum, jejunum, ileum, caecum, and/or colon. In an example, a gastrointestinal electrical stimulator can be configured to be implanted in a subcutaneous site or in an intraperitoneal site. In another example, a gastrointestinal electrical stimulator can be configured to be implanted in adipose tissue or in muscular tissue.

In various examples, a gastrointestinal electrical stimulator can be in electromagnetic communication with a person's vagus nerve, splanchnic nerve, geniculate ganglion, petrosal ganglion, and/or other portions of a person's nervous system that innervate the person's gastrointestinal organs. In various examples, a gastrointestinal electrical stimulator can deliver a neurostimulating or neuroblocking electrical charge or pulse to a person's vagus nerve, splanchnic nerve, geniculate ganglion, petrosal ganglion, and/or other portions of a person's nervous system that innervate the person's gastrointestinal organs. In various examples, a gastrointestinal electrical stimulator can activate, increase, decrease, or block neural signals traveling through a person's vagus nerve, splanchnic nerve, geniculate ganglion, petrosal ganglion, and/or other portions of a person's nervous system that innervate the person's gastrointestinal organs.

In various examples, an implanted gastrointestinal electrical stimulator can: deliver an electrical charge, pulse, or signal to a gastrointestinal organ or to a nerve that innervates such an organ; deliver an electrical charge, pulse, or signal to a person's mouth, tongue, esophagus, stomach, duodenum, pancreas, and/or liver; and/or deliver an electrical charge, pulse, or signal to a person's vagus nerve, splanchnic nerve, geniculate ganglion, and/or petrosal ganglion. In various examples, an implanted gastrointestinal electrical stimulator can: expose a gastrointestinal organ or a nerve that innervates such an organ to an electromagnetic field; expose a person's mouth, tongue, esophagus, stomach, duodenum, pancreas, and/or liver to an electromagnetic field; and/or expose a person's vagus nerve, splanchnic nerve, geniculate ganglion, and/or petrosal ganglion to an electromagnetic field.

In various examples, an implanted gastrointestinal electrical stimulator can: electrically stimulate a gastrointestinal organ or to a nerve that innervates such an organ; electrically stimulate a person's mouth, tongue, esophagus, stomach, duodenum, pancreas, and/or liver; and/or electrically stimulate a person's vagus nerve, splanchnic nerve, geniculate ganglion, and/or petrosal ganglion. In various examples, an implanted gastrointestinal electrical stimulator can: electrically inhibit or otherwise alter the natural electrical activity of a gastrointestinal organ or to a nerve that innervates such an organ; electrically inhibit or otherwise alter the natural electrical activity of a person's mouth, tongue, esophagus, stomach, duodenum, pancreas, and/or liver; and/or electrically inhibit or otherwise alter the natural electrical activity of a person's vagus nerve, splanchnic nerve, geniculate ganglion, and/or petrosal ganglion.

In various examples, an implanted gastrointestinal electrical stimulator can: provide neuromodulation for one or more nerves that are in electrochemical communication with a person's gastrointestinal tract; provide neuromodulation for one or more nerves that are in electrochemical communication with a person's oral cavity and/or nasal cavity; provide neuromodulation for a person's vagus nerve, splanchnic nerve, geniculate ganglion, and/or petrosal ganglion; and/or provide neuromodulation for one or more nerves in a person's parasympathetic nervous system. In various examples, an implanted gastrointestinal electrical stimulator can: comprise a macrostimulator member; comprise a microstimulator member; comprise a neurostimulator member; comprise a neuromodulator member; comprise a neuroblocker member; and/or comprise a gastric pacemaker.

In an example, an implanted gastrointestinal electrical stimulator can modify a person's sense of taste by stimulating or blocking nerves that innervate the person's taste buds. In an example, an implanted gastrointestinal electrical stimulator can modify a person's sense of smell by stimulating or blocking nerves that innervate the person's olfactory cells. In an example, a gastrointestinal electrical stimulator can reduce a person's food consumption by temporarily decreasing their sense of taste and/or sense of smell. In an example, a gastrointestinal electrical stimulator can cause temporary ageusia. In an example, a gastrointestinal electrical stimulator can cause temporary anosmia. In an example, a gastrointestinal electrical stimulator can cause a person to experience a temporary virtual taste or virtual smell by stimulating nerves associated with the person's sense of taste or sense of smell. In an example, this temporary virtual taste or virtual smell can be unpleasant and cause a person to consume less food.

In various examples, a gastrointestinal electrical stimulator can: create a sense of gastric fullness for a person; create a sense of food satiety for a person; mimic a person's natural sensation of satiety and/or fullness after a meal; reduce the amount of food that a person consumes by creating a feeling of gastric fullness; reduce the amount of food that a person consumes by creating a feeling of food satiety; and/or reduce the amount of food that a person consumes by reducing a person's appetite. In various examples, an implanted gastrointestinal electrical stimulator can: contract the pyloric sphincter; delay or slow gastric emptying; slow the passage of food from the stomach into the duodenum; induce temporary gastroparesis; and/or promote gastric distention. In various examples, a gastrointestinal electrical stimulator can: reduce the amount of food that a person consumes by decreasing the speed at which food moves through a portion of a person's gastrointestinal tract; and/or reduce the amount of food that a person consumes by decreasing the passage of food out of the stomach through the pyloric sphincter and causing expansion of stomach walls. In various examples, a gastrointestinal electrical stimulator can: reduce gastric peristalsis; increase gastric peristalsis; reverse the direction of gastric peristalsis; create anti-peristaltic gastric waves which decrease a person's food intake; and/or change the mobility of chyme from the stomach fundus to the stomach antrum.

In various examples, a gastrointestinal electrical stimulator can: reduce absorption of nutrients from food that a person consumes by increasing the speed at which food moves through a portion of a person's gastrointestinal tract; reduce absorption of nutrients from food that a person consumes by decreasing the time during which food mixes with gastric fluids in the stomach; and/or reduce absorption of nutrients from food that a person consumes by decreasing the time of fluid communication between chyme and the walls of a person's duodenum. In various examples, a gastrointestinal electrical stimulator can: reduce the flow of saliva into a person's gastrointestinal tract; reduce the flow of gastric secretions into a person's gastrointestinal tract; reduce the flow of biliary secretions into a person's gastrointestinal tract; and/or reduce the flow of pancreatic secretions into a person's gastrointestinal tract. In various examples, a gastrointestinal electrical stimulator can: reduce the amount of food that a person consumes by delivering an unpleasant electrical stimulus to a person's body in response to food consumption; and/or reduce the amount of food that a person consumes by delivering an electrical stimulus to a person's body that causes nausea and/or discomfort.

In various examples, adjustable parameters of an electrical charge, pulse, or signal created by a gastrointestinal electrical stimulator can be selected from the group consisting of: wave form (e.g. sinusoidal, saw-tooth, or square), ramp rate, duration, continuity, periodicity, randomization, frequency, pulse width, voltage, and amplitude. In various examples, the wave form, duration, continuity, frequency, and/or amplitude of the electricity delivered by a gastrointestinal electrical stimulator to a gastrointestinal organ (or a nerve innervating such an organ) can be adjusted based on one or more factors selected from the group consisting of: the amount and/or duration of consumption of a specific nutrient or nutrient type; a person's recent patterns of nutrient consumption; the speed or pace of nutrient consumption; the time of day; the day of the week; changes in a person's weight; a person's general health status indicators; and a person's exercise patterns and/or caloric expenditure. In an example, this adjustment can be done manually. In an example, this adjustment can occur automatically.

In an example, a gastrointestinal electrical stimulator can include: a microprocessor or Central Processing Unit (CPU); a memory; a wireless communications member for receiving and transmitting wireless data; a power source; and an electrical-energy-delivering component. In an example, there can be wireless communication between a gastrointestinal electrical stimulator and other components of this invention if these components are in different locations. In an example, components sharing wireless communication can include: a specific-nutrient-identifying sensor; a gastrointestinal electrical stimulator; a cumulative-nutrient-consumption regulator; and a generic-food-consumption monitor. In various examples, a gastrointestinal electrical stimulator can be made with one or more materials from the group consisting of: cobalt-chromium alloy, Dacron, fluoropolymer, glass, liquid-crystal polymer, nitinol, nylon, perflouroethylene, platinum, polycarbonate, polyester, polyethylene, polyolefin, polypropylene, polystyrene, polytetrafluoroethylene (PTFE), polyurethane, pyrolytic carbon, silicon, silicone, silk, stainless steel, tantalum, titanium, and urethane. In various examples, a gastrointestinal electrical stimulator can be co-located with a generic-food-consumption monitor, specific-nutrient-identifying sensor, or cumulative-nutrient-consumption regulator.

In various examples, a gastrointestinal electrical stimulator can be powered by an internal power source, by an external power source, or by a combination of internal and external power sources. In an example, a power source can be charged from an external source by electromagnetic inductance. In an example, a power source can be charged by harvesting mechanical, thermal, chemical, or biological energy from a person's body. In an example, a power source can be a rechargeable battery, microchip, or capacitor. In various examples, a gastrointestinal electrical stimulator can transduce kinetic, thermal, or biochemical energy from a person's body. In various examples, a gastrointestinal electrical stimulator can be powered from one or more energy sources selected from the group consisting of: a battery, an energy-storing chip, energy harvested or transduced from a bioelectrical cell, energy harvested or transduced from an electromagnetic field, energy harvested or transduced from an implanted biological source, energy harvested or transduced from blood flow or other internal fluid flow, energy harvested or transduced from body kinetic energy, energy harvested or transduced from glucose metabolism, energy harvested or transduced from muscle activity, energy harvested or transduced from organ motion, and energy harvested or transduced from thermal energy.

FIGS. 1 through 8 show various examples of how this invention can be embodied in a device and method that includes a cumulative-nutrient-consumption regulator. We now discuss, in greater detail and example variation, how a cumulative-nutrient-consumption regulator can be configured and can function.

This invention can be embodied in a device and method that includes a cumulative-nutrient-consumption regulator. A cumulative-nutrient-consumption regulator: (a) can keep track of the cumulative amount and/or duration of a person's consumption of at least one selected nutrient, wherein this specific nutrient is detected by a specific-nutrient-identifying sensor; and (b) can trigger a gastrointestinal electrical stimulator when the cumulative amount and/or duration of consumption of this selected nutrient exceeds a pre-determined allowable amount and/or duration of consumption for this nutrient.

In an example, a cumulative-nutrient-consumption regulator allows normal consumption of a healthy nutrient and allows normal consumption of up to an allowable amount of an unhealthy nutrient. However, it reduces consumption and/or absorption of an excessive amount (over an allowable amount) of an unhealthy nutrient. In an example, a cumulative-nutrient-consumption regulator can allow normal consumption of healthy food and allow normal consumption up to an allowable amount of unhealthy food, but it can reduce consumption and/or absorption of an excessive amount of unhealthy food. In an example, a cumulative-nutrient-consumption regulator allows a healthy amount of food to be consumed and digested in a normal manner, but modifies the consumption and/or digestion of an unhealthy amount of food.

The device and method disclosed herein provides specific-nutrient discrimination and limitation capability. This is superior to bariatric surgery and malabsorption devices in the prior art that are blind to whether a certain bolus of food traveling through the gastrointestinal tract is healthy or unhealthy. This device and method avoids the deficiencies of essential nutrients that can occur with food-blind malabsorption procedures and devices in the prior art. This device and method can be a key part of an overall system to ensure that a person receives proper nutrition, even while this person is losing weight. A weight management system incorporating such a cumulative-nutrient-consumption regulator can help a person to lose weight without the nutritional deficiencies that can be caused by nutrient-blind bariatric procedures and devices in the prior art.

In various examples, an allowable amount of consumption and/or duration of consumption for a selected nutrient can be based, in whole or in part, on one or more factors selected from the group consisting of: a dietary plan created for a person by a health care provider; recommendations or medical orders from a health care provider; input from a virtual health coach; and input from a social network or behavioral support group.

In various examples, an allowable amount and/or duration of consumption for a selected nutrient can be based, in whole or in part, on one or more factors selected from the group consisting of: an allowable amount of a selected nutrient per meal; an allowable amount of a selected nutrient per day; an allowable amount of a selected nutrient per week; a rolling average allowable consumption amount based on multiple sequential minutes, hours, or days; a person's recent pattern of eating and nutrient consumption; and a person's speed or pace of eating and nutrient consumption.

In various examples, an allowable amount and/or duration of consumption for a selected nutrient can be based, in whole or in part, on one or more factors selected from the group consisting of: the time of day (e.g. to discourage nocturnal eating binges or between-meal snacking); the day of the week; and the occurrence of a holiday or other occasion involving special meals.

In various examples, an allowable amount and/or duration of consumption for a selected nutrient can be based, in whole or in part, on one or more factors selected from the group consisting of: a person's age, gender, weight, and/or anatomy; changes in a person's weight; a person's diagnosed health conditions; indicators of a person's general health status; a person's exercise patterns and/or caloric expenditure; and achievement of a person's health goals.

In various examples, an allowable amount and/or duration of consumption for a selected nutrient can be based, in whole or in part, on one or more factors selected from the group consisting of: the median or mean consumption amount of the selected nutrient for the general population; recommended consumption amount of the nutrient for the general population; the allowable amount of a selected nutrient in a specific low-carbohydrate, low-fat, or high-protein diet; the amount and/or duration of a person's consumption of healthy food or nutrients; and the amount of healthy food consumed in conjunction with unhealthy food.

In various examples, an allowable amount and/or duration of consumption for a selected nutrient can be based, in whole or in part, on one or more factors selected from the group consisting of: the type of selected nutrient; the specificity or breadth of the selected nutrient type; and the accuracy of the specific-nutrient-identifying sensor in detecting consumption of the selected nutrient; the magnitude and/or certainty of the relationship between a person's past consumption of the selected nutrient and the person's health indicators.

In various examples, the allowable amount and/or duration of consumption for a selected nutrient can be based, in whole or in part, on one or more factors selected from the group consisting of: the cost of food; financial payments, constraints, and/or incentives; health insurance copays and/or health insurance premiums; and a person's physical location (e.g. proximity to places where unhealthy food tends to be consumed).

In an example, a cumulative-nutrient-consumption regulator can trigger the start of delivery of electricity by a gastrointestinal electrical stimulator when an allowable amount and/or duration of nutrient consumption for a selected nutrient is exceeded. In an example, a cumulative-nutrient-consumption regulator can trigger the end of such delivery of electricity after a predetermined period of time. In an example, a cumulative-nutrient-consumption regulator can trigger the end of this delivery of electricity based on information from a specific-nutrient-identifying sensor or from a generic-food-consumption monitor.

In an example, a cumulative-nutrient-consumption regulator can trigger the end of such delivery of electricity when there is a change in one or more parameters associated with a person's gastrointestinal tract. In an example, a specific-nutrient-identifying sensor, a gastrointestinal electrical stimulator, and a cumulative-nutrient-consumption regulator can comprise a closed-loop system for selective detection of a specific nutrient and reduction of consumption and/or absorption of that specific nutrient.

In an example, a cumulative-nutrient-consumption regulator can be used to adjust the delivery parameters of the electricity that is delivered by a gastrointestinal electrical stimulator to a gastrointestinal organ and/or to a nerve that innervates such an organ. In various examples, electricity parameters that can be adjusted can be selected from the group consisting of: the wave form, the duration, the continuity, the frequency, and/or the amplitude.

In various examples, one or more of these parameters can be adjusted based on one or more factors selected from the group consisting of: a person's past responses to gastrointestinal electrical stimulation; recent changes in a person's food consumption; recent changes in a person's exercise patterns and/or caloric expenditure; recent changes in a person's weight; recent changes in indicators of a person's general health status; the time of day; and the day of the week. In an example, such adjustments can be made manually. In an example, such adjustments can occur automatically.

In various examples, a cumulative-nutrient-consumption regulator can be used to automatically adjust the wave form, duration, continuity, frequency, and/or amplitude of the electricity that is delivered by a gastrointestinal electrical stimulator based on one or more factors selected from the group consisting of: the amount and/or duration of a person's consumption of healthy food or nutrients; the amount of healthy food consumed in conjunction with unhealthy food; the accuracy of a sensor in detecting consumption of a selected nutrient; and the magnitude and/or certainty of the effects of past consumption of the selected nutrient on a person's health.

In an example, a cumulative-nutrient-consumption regulator combined with a specific-nutrient-identifying sensor can selectively detect cumulative consumption of food during a period of time that exceeds an allowable number of grams of fat, an allowable number of grams of saturated fat, an allowable number of milligrams of fat cholesterol, an allowable number of grams of carbohydrates, and/or an allowable number of milligrams of sodium.

In an example, a cumulative-nutrient-consumption regulator combined with a specific-nutrient-identifying sensor can identify unhealthy types of food based on such food having one or more nutritional quantities selected from the group consisting of: having at least an allowable number of grams of fat per suggested serving, having at least an allowable number of grams of saturated fat per suggested serving, having at least an allowable number of milligrams of fat cholesterol per suggested serving, having at least an allowable number of grams of carbohydrates per suggested serving, and/or having at least an allowable number of milligrams of sodium per suggested serving.

In an example, a cumulative-nutrient-consumption regulator combined with a specific-nutrient-identifying sensor can identify unhealthy types of food based on such food exceeding one or more nutritional percentages selected from the group consisting of: an allowable percentage of the recommended daily intake of fat per suggested serving, an allowable percentage of the recommended daily intake of saturated fat per suggested serving, an allowable percentage of the recommended daily intake of fat cholesterol per suggested serving, an allowable percentage of the recommended daily intake of carbohydrate per suggested serving, and/or an allowable percentage of the recommended daily intake of sodium per suggested serving.

In an example, there can be a pre-determined list of selected nutrients or foods (stored within a microprocessor within a cumulative-nutrient-consumption regulator) which trigger activation of a gastrointestinal electrical stimulator when these nutrients or foods are eaten in excessive amounts. In an example, a list of the types of nutrients or foods containing these nutrients can be modified in a non-invasive manner. In an example, allowable quantities of nutrients or foods can be changed by programming. In an example, a cumulative-nutrient-consumption regulator can be programmed to modify this list in an automatic manner. In an example, the types of nutrients and/or allowable amounts of these nutrients can be automatically modified by an information processor with automatic learning capability.

In an example, there can be a predefined list of nutrient types and/or food types (containing those nutrients) which are classified as unhealthy. In an example, there can be predefined consumption amounts of nutrients and/or foods containing those nutrients which are classified as excessive and unhealthy. In an example, lists of the types and/or quantities of nutrients and/or foods which are classified as unhealthy can be compiled and adjusted by experts and professionals who provide a person with nutritional and dietary counseling.

In an example, a cumulative-nutrient-consumption regulator can be implanted within a person or within part of a device that is implanted within a person. In an example, a cumulative-nutrient-consumption regulator can be external to a person's body and in wireless communication with devices that are implanted within the person. In various examples, a cumulative-nutrient-consumption regulator can be co-located with a nutrient-specific sensor, a gastrointestinal electrical stimulator, and/or a generic-food-consumption monitor. In various examples, a cumulative-nutrient-consumption regulator can work alone or in combination with a remotely-located computer with which the regulator is in communication.

In an example, healthy types of nutrients and/or food can be identified in a negative manner—as any type of nutrient or food that is not positively identified as being unhealthy. In an alternative example, healthy types of nutrients and/or food can be identified in a positive manner—as food with a large concentration or amount of selected good nutrients. In various examples, good nutrients or foods can be characterized by one or more factors selected from the group consisting of: having a high amount and/or concentration of soluble fiber, having a high amount and/or concentration of insoluble fiber, having a high amount and/or concentration of essential vitamins, having a high amount and/or concentration of protein, and having a high amount and/or concentration of essential nutrients that a person's diet generally lacks.

In various examples, a cumulative-nutrient-consumption regulator can be made with one or more materials from the group consisting of: cobalt-chromium alloy, Dacron, fluoropolymer, glass, liquid-crystal polymer, nitinol, nylon, perflouroethylene, platinum, polycarbonate, polyester, polyethylene, polyolefin, polypropylene, polystyrene, polytetrafluoroethylene (PTFE), polyurethane, pyrolytic carbon, silicon, silicone, silk, stainless steel, tantalum, titanium, and urethane.

In various examples, a cumulative-nutrient-consumption regulator can include: an electronic or optical microprocessor and/or Central Processing Unit (CPU); a communications component that can transmit and/or receive data in a wireless manner; a memory component to store information concerning specific nutrients, foods, and/or a person's cumulative consumption of those nutrients and/or foods; and a power source.

In an example, a cumulative-nutrient-consumption regulator can include a memory that tracks the cumulative amounts of selected nutrients and/or foods that a person consumes during an episode of eating (e.g. during a meal) or during a selected period of time (e.g. during a day). For example, a cumulative-nutrient-consumption regulator may track how many units of sugar, fat, or sodium are consumed by a person during the course of a meal or during the span of a day. In an example, a cumulative-nutrient-consumption regulator can track and record cumulative consumption of one or more specific nutrients during an eating episode or during a period of time based on information received from a specific-nutrient-identifying sensor. In an example, an eating episode can be defined as a period of time with continuous eating. In an example, an eating episode can be defined as a period of time with less than a selected amount of time between mouthfuls and/or swallows.

In an example, a device can be programmed to allow unmodified consumption and/or absorption of nutrients for up to an allowable amount or up to an allowable duration. In an example, a device can allow up to a certain amount of one or more selected types of nutrients and/or foods to be consumed by a person before food consumption and/or absorption is modified by a gastrointestinal electrical stimulator. In an example, a cumulative-nutrient-consumption regulator can use information from one or more food consumption sensors to automatically allow up to a selected amount of nutrients and/or food to be consumed normally during a given eating episode or time period.

In an example, a cumulative-nutrient-consumption regulator can be programmed to allow moderate consumption of some nutrients or foods without modifying consumption or digestion, but can modify consumption and/or digestion if there is excessive consumption of those nutrients or foods. In an example, a device can start modifying consumption or digestion of a selected type of nutrient or food when a sensor detects that a person has consumed an excessive amount of an unhealthy nutrient or food and can stop this modification when the sensor detects that a person has begun consuming a healthy nutrient or food. In an example, a cumulative-nutrient-consumption regulator can use information from a nutrient-specific sensor in order to modify consumption of excess nutrient and/or food consumption.

In an example, a cumulative-nutrient-consumption regulator (or a microprocessor or CPU within it) can be adjusted and/or programmed post-operatively in a minimally-invasive and reversible manner. In an example, a cumulative-nutrient-consumption regulator can communicate wirelessly with a remote control unit that is external to a person's body. In an example, a cumulative-nutrient-consumption regulator can itself be external to a person's body and communicate wirelessly with components that are implanted with the person's body. In an example, a cumulative-nutrient-consumption regulator can be programmed, or otherwise adjusted, by an external remote control unit. In an example, a cumulative-nutrient-consumption regulator can be wirelessly programmed, or otherwise adjusted, by the person in whom the device is implanted. In an example, a cumulative-nutrient-consumption regulator can be wirelessly programmed, or otherwise adjusted, by an informal care giver or by a health care professional.

In various examples, a cumulative-nutrient-consumption regulator (or a microprocessor or CPU within it) can be adjusted and/or programmed to change one or more of the following aspects of its functioning: the allowable amounts of cumulative consumption for one or more selected nutrients, selected nutrient types, and/or selected food types before it triggers a gastrointestinal electrical stimulator to deliver electricity to a gastrointestinal organ and/or a nerve innervating such an organ; the time of day, day of the week, or other timing parameter wherein consumption of a selected nutrient triggers delivery of electricity to a gastrointestinal organ and/or a nerve innervating such an organ; the effect of a person's past food consumption and/or caloric expenditure on delivery of electricity to a gastrointestinal organ and/or a nerve innervating such an organ; the effect of a person's physical location (as measured by a GPS) on delivery of electricity to a gastrointestinal organ and/or a nerve innervating such an organ; the effect of special social events and holidays on delivery of electricity to a gastrointestinal organ and/or a nerve innervating such an organ; the effect of a personalized diet plan (such as one created by a health care professional) on delivery of electricity to a gastrointestinal organ and/or a nerve innervating such an organ; and the effect of social networking connections and support groups on delivery of electricity to a gastrointestinal organ and/or a nerve innervating such an organ.

In an example, there can be wireless communication among different components of this device if these components are located in different places. In an example, components sharing wireless communication can include: a specific-nutrient-identifying sensor; a gastrointestinal electrical stimulator; and a cumulative-nutrient-consumption regulator. In another example, components sharing wireless communication can include: a generic-food-consumption monitor; a specific-nutrient-identifying sensor; a gastrointestinal electrical stimulator; and a cumulative-nutrient-consumption regulator.

In various examples, a cumulative-nutrient-consumption regulator can be in wireless communication with one or more of the following members: a sensor that is implanted within, or attached to, a different location within a person's body; a remote computer, network, or control unit that is external to a person's body; and an external mobile, cellular, or tabular electronic communication device. In various examples, any or all of the components of this device may share wireless communication with a remote control unit or other mobile electronic device that is external to a person's body.

In an example, a cumulative-nutrient-consumption regulator can communicate wirelessly with a remote control unit that is external to a person. In an example, a cumulative-nutrient-consumption regulator can communicate wirelessly with one or more devices or systems selected from the group consisting of: a smart phone, a mobile and/or cellular phone, a pedometer or other motion-tracking device, a piece of electronically-functional jewelry, a portable electronic device, an external sensor that is worn on the body, a laptop computer, a desktop computer, a remote server, a social network system, an electronic tablet or pad, and the internet.

In an example, a cumulative-nutrient-consumption regulator can use information received from one or more nutrient and/or food consumption sensors to adjust the types and/or amounts of nutrients and/or food that trigger electrical stimulation of gastrointestinal organs and/or nerves that innervate such organs. In an example, one or more sensors may be co-located with the regulator. In an example, one or more sensors may be located elsewhere and in wireless communication with the regulator. In an example, one or more of these components may share electromagnetic communication through wires or electronic circuits. Since there are many types of wires and electronic circuits in the prior art and since the precise pathways for such connections are not central to this invention, wire connections are not shown in the figures to avoid cluttering them.

In various examples, a cumulative-nutrient-consumption regulator can be powered by energy that is harvested, transduced, generated, and/or converted from kinetic, thermal, or biochemical energy within a person's body. In various examples, a cumulative-nutrient-consumption regulator can be powered from one or more energy sources selected from the group consisting of: a battery, an energy-storing chip, energy harvested or transduced from a bioelectrical cell, energy harvested or transduced from an electromagnetic field, energy harvested or transduced from an implanted biological source, energy harvested or transduced from blood flow or other internal fluid flow, energy harvested or transduced from body kinetic energy, energy harvested or transduced from glucose metabolism, energy harvested or transduced from muscle activity, energy harvested or transduced from organ motion, and energy harvested or transduced from thermal energy.

FIGS. 7 through 8 show some examples of how this invention can be embodied in a device and method that includes a generic-food-consumption monitor. We now discuss, in greater detail and example variation, how a generic-food-consumption monitor can be configured and can function.

In an example, this invention can be embodied in a device and method that includes a generic-food-consumption monitor in addition to a specific-nutrient-identifying sensor. In an example, a generic-food-consumption monitor can operate in a more-continuous manner than a specific-nutrient-identifying sensor, but the generic-food-consumption monitor has less ability to detect consumption of specific nutrients than does a specific-nutrient-identifying sensor. In an example, the operation of a specific-nutrient-identifying sensor can be triggered by food consumption as indicated by a generic-food-consumption monitor. In an example, a generic-food-consumption monitor and a specific-nutrient-identifying sensor can operate together to provide greater nutrient discrimination ability, in a more-efficient and less-invasive manner, than either member working alone.

In an example, a generic-food-consumption monitor can monitor a person's behavior and/or gastrointestinal tract in a long-term ongoing manner in order to detect when there is a high probability that the person is consuming food. In an example, a generic-food-consumption monitor can detect when a person is consuming food, but is limited in its ability to selectively differentiate consumption of a specific nutrient type or food type. In an example, a generic-food-consumption monitor can detect that a person is probably eating food of some type, but cannot identify what type of food the person is eating (beyond very general parameters such as whether the food is relatively solid or liquid). In an example, a generic-food-consumption monitor does not have the nutrient discriminatory ability to selectively detect when a person is consuming food with one or more specific nutrients selected from the group consisting of: a type of sugar, a type of carbohydrate, a type of fat, a type of cholesterol, and a sodium compound.

In an example, a generic-food-consumption monitor can operate in a more-continuous and/or more-continual ongoing manner than a consumption sensor. In an example, a generic-food-consumption monitor can continuously, continually, or constantly monitor a person's behavior and/or gastrointestinal tract in order to detect food consumption. In an example, a generic-food-consumption monitor can monitor a person's behavior and/or gastrointestinal tract according to a pre-determined non-continuous sensing schedule in order to be more-efficient and/or less-invasive than continuous monitoring. In an example, a monitoring schedule can comprise periodic sensing, sampling, or testing of the person's actions and/or material in the person's gastrointestinal tract. In an example, a generic-food-consumption monitor can monitor a person's behavior and/or gastrointestinal tract with random sampling of behavioral and/or gastrointestinal activity.

In an example, a generic-food-consumption monitor can work in conjunction with a specific-nutrient-identifying sensor. In an example, a generic-food-consumption monitor can operate in a more-continuous manner to detect general food consumption, a specific-nutrient-identifying sensor can operate in a less-continuous manner to detect consumption of a specific nutrient, and operation of the specific-nutrient-identifying sensor can be triggered, activated, and/or augmented when general food consumption is indicated by the generic-food-consumption monitor. Such cooperative operation of a more-continuous generic-food-consumption monitor and a less-continuous specific-nutrient-identifying sensor can selectively identify consumption of specific nutrients more efficiently, with lower energy requirements, and with less privacy intrusion than is possible with either the monitor or the sensor operating in isolation. In an example, a generic-food-consumption monitor can be co-located with a specific-nutrient-identifying sensor. In an example, a generic-food-consumption monitor can be located in a different place than a specific-nutrient-identifying sensor.

In various examples, a generic-food-consumption monitor can be selected from the group consisting of: biological monitor, enzyme-based monitor, protein-based monitor, and/or reagent-based monitor; camera, imaging monitor, and/or pattern recognition monitor; chemical monitor, biochemical monitor, chemoreceptor, osmolality monitor, and/or pH level monitor; electromagnetic monitor, EGG monitor, EMG monitor, impedance monitor, interferometer, muscle activity monitor, and/or neural impulse monitor; glucose monitor, fat monitor, cholesterol monitor, amino acid monitor, and/or micronutrient monitor; Micro Electrical Mechanical System (MEMS) monitor, microfluidic monitor, laboratory-on-a-chip, medichip, and/or membrane-based monitor; motion monitor, movement monitor, accelerometer, flow monitor, strain gauge, electrogoniometer, and/or peristalsis monitor; optical monitor, optoelectronic monitor, infrared monitor, spectroscopy monitor, and/or chromatography monitor; pressure monitor; sound monitor, acoustic energy monitor, microphone, chewing monitor, swallow detector, and/or ultrasonic monitor; taste monitor, olfactory monitor, and/or electronic nose; and temperature monitor, and/or thermistor.

In an example, a generic-food-consumption monitor can be implanted within a person's body. There are advantages to having a generic-food-consumption monitor be implanted within a person's body. In an example of such an advantage, an implanted generic-food-consumption monitor can be more consistent and automatic in its monitoring function than an external sensor. In an example of such an advantage, an implanted generic-food-consumption monitor can be less prone to compliance problems or circumvention by the person whose food consumption is being monitored. An implanted sensor is less dependent on voluntary action by the person being monitored. As another advantage, an implanted generic-food-consumption monitor can provide some types of information concerning food consumption which are more difficult to obtain using an external sensor. For example, an implanted sensor can provide monitoring based on direct fluid and/or electromagnetic communication with material in the person's gastrointestinal tract.

In an example, a generic-food-consumption monitor can be implanted anywhere within a person's body that is in fluid, chemical, electromagnetic, neural, optical, acoustic, mechanical, or other sensory communication with one or more organs along a person's gastrointestinal tract or a nerve that innervates such an organ. In various examples, a generic-food-consumption monitor can be implanted within, attached to, or otherwise in sensory communication with a person's oral cavity, nasal cavity, esophagus, stomach, duodenum, or other portions of a person's intestine. In various examples, a generic-food-consumption monitor can be implanted within, or attached to, a secretory organ such as the pancreas or liver that is in fluid communication with a person's gastrointestinal tract. In various examples, a generic-food-consumption monitor can be in electromagnetic communication with a nerve that innervates an organ along a person's gastrointestinal tract. In various examples, a generic-food-consumption monitor can be in electromagnetic communication with a person's vagus nerve, splanchnic nerve, geniculate ganglion, and/or petrosal ganglion.

In various examples, a generic-food-consumption monitor can be configured to be implanted within a person's oral cavity and/or nasal cavity in a location selected from the group consisting of: attached to a person's palatal vault and/or upper roof of their mouth; attached to a person's teeth, such as the buccal spaces, with an adhesive, band, wire, or other fastening mechanism; attached or implanted within a person's mouth using a bio-adhesive, bone screw, or other fastening mechanism; implanted within a person's teeth or within a dental or orthodontic prosthesis such as a retainer, dentures, bridge, cap, or crown; implanted into a person's soft palatal tissue; implanted into a person's tongue; implanted within a person's body so as to be in electromagnetic communication with nerves that innervate a person's tongue; implanted within a person's body so as to be in electromagnetic communication with the geniculate ganglion and/or petrosal ganglion; implanted within the sublingual space of a person's oral cavity; implanted within a person's body so as to be in fluid communication with the oral cavity via an artificial lumen; and implanted within a person's nasal cavity so as to be in fluid and/or gaseous communication with a person's oral cavity.

In an example, an intraoral generic-food-consumption monitor can be flexible, have a smooth exterior, and be shaped to match the contour of underlying oral tissue in order to minimize tissue irritation with a person's mouth. In an example, such a generic-food-consumption monitor can be configured so as not to interfere with a person's eating, talking, and breathing. In an example, an intraoral monitor can be sealed to protect its internal components from oral fluids, with the possible exception of a controllable opening, membrane, and/or filter for sampling food, saliva, or other oral fluids into a reservoir for analysis.

In various examples, an intraoral generic-food-consumption monitor can take and analyze samples of material from within a person's oral cavity using one or more mechanisms selected from the group consisting of: biochemical specimen analysis; electrochemical specimen analysis; electroosmotic sampling; electrophoresis; electroporation; enzymatic specimen analysis; MEMS-based specimen analysis; microfluidic specimen analysis; piezoelectric specimen analysis; and spectroscopic specimen analysis.

In various examples, a generic-food-consumption monitor can monitor one or more portions of a person's gastrointestinal tract and/or nerves innervating the gastrointestinal tract. In an example, a generic-food-consumption monitor can monitor for swallowing and/or chewing sounds. In an example, a generic-food-consumption monitor can monitor the electrical impedance of a portion of a person's gastrointestinal tract and/or material passing through the gastrointestinal tract. In an example, a generic-food-consumption monitor can perform infrared spectroscopy on material in a person's oral cavity and/or gastrointestinal tract. In an example, a generic-food-consumption monitor can monitor the electromagnetic activity of gastrointestinal organs and/or nerves that innervate these organs. In an example, a generic-food-consumption monitor can monitor the wall movement, peristaltic motion, internal pressure, and/or external pressure of a gastrointestinal organ.

In various examples, a generic-food-consumption monitor can be implanted so as to be in fluid, chemical, electromagnetic, neural, optical, acoustic, mechanical, or other sensory communication with a person's esophagus, including their Lower Esophageal Sphincter (LES) and/or Upper Esophageal Sphincter (UES). In an example, a generic-food-consumption monitor can monitor the activity of a person's esophagus, including their Lower Esophageal Sphincter (LES) and/or Upper Esophageal Sphincter (UES).

In various examples, a generic-food-consumption monitor can be implanted so as to be in fluid, chemical, electromagnetic, neural, optical, acoustic, mechanical, or other sensory communication with a person's stomach, including the cardia, pyloric sphincter, fundus, lesser curvature, and/or greater curvature. In an example, a generic-food-consumption monitor can monitor the activity of a person's stomach, including the cardia, pyloric sphincter, fundus, lesser curvature, and/or greater curvature.

In various examples, a generic-food-consumption monitor can be implanted so as to be in fluid, chemical, electromagnetic, neural, optical, acoustic, mechanical, or other sensory communication with a person's duodenum and/or other portions of a person's intestine. In an example, a generic-food-consumption monitor can monitor the activity of a person's duodenum and/or other portions of a person's intestine.

In various examples, a generic-food-consumption monitor can be implanted so as to be in fluid, chemical, electromagnetic, neural, optical, acoustic, mechanical, or other sensory communication with a person's pancreas or liver. In an example, a generic-food-consumption monitor can monitor the activity of a person's pancreas or liver.

In various examples, a generic-food-consumption monitor can be implanted so as to be in electromagnetic, electrochemical, and/or neural communication with a person's vagus nerve, splanchnic nerve, geniculate ganglion, petrosal ganglion, and/or other portions of a person's nervous system that innervate a person's gastrointestinal organs. In an example, a generic-food-consumption monitor can monitor the electromagnetic and/or electrochemical activity of a person's vagus nerve, splanchnic nerve, geniculate ganglion, petrosal ganglion, and/or other portions of a person's nervous system that innervate a person's gastrointestinal organs.

In an example, a generic-food-consumption monitor can be in electromagnetic communication with electrodes implanted within brain tissue, wherein electromagnetic signals from this brain tissue provide an indication of food consumption. In an example, a generic-food-consumption monitor can be in electromagnetic communication with electrodes implanted within a person's hypothalamus.

In various examples, a generic-food-consumption monitor can be implanted within a person's abdominal cavity with a means of fluid, neural, or other communication with a person's stomach, duodenum, jejunum, ileum, caecum, colon, or esophagus. In an example, a generic-food-consumption monitor can be configured to be implanted in a subcutaneous site or in an intraperitoneal site. In another example, a generic-food-consumption monitor can be configured to be implanted in adipose tissue or in muscular tissue.

In various examples, an implanted generic-food-consumption monitor can detect food consumption by a mechanism selected from the group consisting of: acoustic monitoring for swallowing and/or chewing sounds from a person (such as with a microphone); acoustic monitoring of a person's oral cavity and/or gastrointestinal organs (such as with a microphone); and ultrasonic monitoring of a person's oral cavity and/or gastrointestinal organs or material within them (such as with an ultrasound emitter).

In various examples, an implanted generic-food-consumption monitor can detect food consumption by a mechanism selected from the group consisting of: electromagnetic monitoring of a person's oral cavity and/or gastrointestinal organs or nerves that innervate those organs (such as measurement of gastric electrical signals or esophageal electrical signals using electrodes); impedance monitoring of a person's oral cavity and/or gastrointestinal organs or material within them (such as with electrodes); and neurological monitoring of nerves that innervate a person's gastrointestinal organs (such as the vagus nerve, splanchnic nerve, geniculate ganglion, or petrosal ganglion).

In various examples, an implanted generic-food-consumption monitor can detect food consumption by a mechanism selected from the group consisting of: motion monitoring of a person's oral cavity and/or gastrointestinal organs or material within them (such as measurement of chewing motion, peristaltic motion, wall expansion, or other movements with an accelerometer, strain gauge, or piezoelectric sensor); and pressure monitoring of the interior lumens of a person's gastrointestinal organs (such as intra-gastric or intra-esophageal pressure). In an example, an implanted generic-food-consumption monitor can detect food consumption by thermal monitoring of a person's oral cavity and/or gastrointestinal organs or material within them (such as with a thermometer or thermistor).

In various examples, an implanted generic-food-consumption monitor can detect food consumption by a mechanism selected from the group consisting of: optical monitoring of a person's oral cavity and/or gastrointestinal organs or material within them (such as with a camera, photoelectric eye, infrared spectroscopy, chromatography, or optoelectronic sensor); microfluidic or MEMS monitoring of a person's oral cavity and/or gastrointestinal organs or material within them (such as with a microfluidic laboratory-on-a-chip); and chemical monitoring of a person's oral cavity and/or gastrointestinal organs or material within them (such as measurement of pH level or chemical substances with microfluidic sampling).

In various examples, a gastrointestinal electrical stimulator can be made with one or more materials from the group consisting of: cobalt-chromium alloy, Dacron, fluoropolymer, glass, liquid-crystal polymer, nitinol, nylon, perflouroethylene, platinum, polycarbonate, polyester, polyethylene, polyolefin, polypropylene, polystyrene, polytetrafluoroethylene (PTFE), polyurethane, pyrolytic carbon, silicon, silicone, silk, stainless steel, tantalum, titanium, and urethane.

In various examples, a generic-food-consumption monitor can be powered by an internal power source, by an external power source, or by a combination of internal and external power sources. In an example, a power source can be a rechargeable battery or microchip. In various examples, a generic-food-consumption monitor (or a power source within it) can transduce kinetic, thermal, or biochemical energy from within a person's body.

In various examples, a generic-food-consumption monitor can be powered from one or more energy sources selected from the group consisting of: a battery, an energy-storing chip, energy harvested or transduced from a bioelectrical cell, energy harvested or transduced from an electromagnetic field, energy harvested or transduced from an implanted biological source, energy harvested or transduced from blood flow or other internal fluid flow, energy harvested or transduced from body kinetic energy, energy harvested or transduced from glucose metabolism, energy harvested or transduced from muscle activity, energy harvested or transduced from organ motion, and energy harvested or transduced from thermal energy.

Although there are advantages to having a generic-food-consumption monitor be implanted within a person's body (such as direct communication with a person's oral cavity and/or gastrointestinal tract), in an example a generic-food-consumption monitor can be external to a person's body. In an example, a person can wear an external generic-food-consumption monitor on their body. In an example, a person can wear an external generic-food-consumption monitor on their clothing. In an example, an external generic-food-consumption monitor can be integrated into a particular article of clothing. In an example, an external generic-food-consumption monitor can be incorporated into a mobile electronic device (such as a mobile phone or electronic tablet or touch pad) which a person carries around. In an example, a generic-food-consumption monitor can be integrated into a piece of jewelry worn by a person—such as a wrist watch, bracelet, necklace, pendant, button, or ring.

There can be advantages to having an external generic-food-consumption monitor (as opposed to an implanted one). In an example, an external generic-food-consumption monitor can be less-invasive, safer, and less-costly than an implanted monitor. In an example, an external monitor can detect food-consumption earlier than an implanted monitor. In an example, an external monitor can detect food consumption as a person reaches for food or inserts food into their mouth. As another potential advantage of an external generic-food-consumption monitor, some types of monitoring are easier when performed before food is inserted into a person's mouth. For example, image-based analysis of food consumption can be easier when food is on a plate (or in a labeled container) than after food is chewed within a person's mouth.

In various examples, this invention can be embodied in a device and method comprising an external generic-food-consumption monitor that sends information in a wireless manner to an implanted specific-nutrient-identifying sensor, to an internal cumulative-nutrient-consumption regulator, and/or to a gastrointestinal electrical stimulator. In various examples, this invention can be embodied in a device and method comprising a wearable, portable, and/or mobile generic-food-consumption monitor that sends information in a wireless manner to an implanted specific-nutrient-identifying sensor, to an internal cumulative-nutrient-consumption regulator, and/or to a gastrointestinal electrical stimulator. In an example, a generic-food-consumption monitor can be incorporated into a mobile electronic device, such as a mobile phone or touch pad, that is carried by a person. In an example, an external generic-food-consumption monitoring function can be an application of a mobile electronic device, such as a mobile phone or touch pad. In an example, a generic-food-consumption monitor can communicate wirelessly with the internet.

In an example, a person can wear an external generic-food-consumption monitor. In various examples, a person can wear a generic-food-consumption monitor on their wrist, hand, finger, arm, torso, neck, head, and/or ear. In an example, a generic-food-consumption monitor can be part of a piece of electronically-functional jewelry. In various examples, a generic-food-consumption monitor can be incorporated into one or more of the following wearable members: wrist watch, bluetooth device, bracelet, arm band, button, earring, eyeglasses, finger ring, headphones, hearing aid, necklace, nose ring, and pendant.

In various examples, an external generic-food-consumption monitor can monitor the movements, locations, and/or configurations of a person's arms, hands, wrist, fingers, mouth, and/or head in order to detect food consumption. In various examples, an external generic-food-consumption monitor can use an accelerometer, strain gauge, and/or electrogoniometer to monitor the movements, locations, and/or configurations of a person's arms, hands, wrist, fingers, mouth, and/or head in order to detect food consumption. In an example, an external generic-food-consumption monitor can be worn on a person's wrist in a manner similar to that of a wrist watch or bracelet.

In various examples, an external generic-food-consumption monitor can monitor sounds from a person's mouth, teeth, neck, head, abdomen, and/or torso in order to detect food consumption. In various examples, an external generic-food-consumption monitor can use a microphone to detect chewing and/or swallowing sounds. In an example, an external generic-food-consumption monitor can be embodied in a necklace that detects chewing and/or swallowing sounds. In an example, an external generic-food-consumption monitor with acoustic monitoring capability can be incorporated into an adhesive sensor patch that is worn on the back of a person's neck or on their torso.

In various examples, an external generic-food-consumption monitor can take still and/or moving pictures of a person's hands, arms, head, and/or mouth in order to detect food consumption. In various examples, an external generic-food-consumption monitor can take still and/or moving pictures of the space near a person's hands, arms, head, and/or mouth in order to detect food consumption. In various examples, an external generic-food-consumption monitor can take still and/or moving pictures of the space in front of a person's body in order to detect food consumption. In various examples, an external generic-food-consumption monitor can include a camera that is worn on a person's wrist or around a person's neck. In an example, an external generic-food-consumption monitor can be incorporated into a person's eyeglasses. In an example, an external generic-food-consumption monitor can be incorporated into a contact lens.

As shown by FIGS. 1 through 8, in various examples this invention can be embodied in a device for reducing excess consumption and/or absorption of a selected nutrient, comprising: (a) an intraoral specific-nutrient-identifying sensor; wherein this sensor automatically analyzes the chemical composition of food, saliva, and/or oral fluid in a person's oral cavity in order to selectively identify the person's consumption of at least one selected nutrient; (b) a gastrointestinal electrical stimulator; wherein this stimulator is configured to be implanted within the person's body to deliver electricity to a gastrointestinal organ and/or a nerve that innervates such an organ; and (c) a cumulative-nutrient-consumption regulator; wherein this regulator keeps track of the cumulative amount of consumption and/or duration of consumption of at least one selected nutrient as identified by the specific-nutrient-identifying sensor and triggers the gastrointestinal electrical stimulator to deliver electricity to a gastrointestinal organ and/or to a nerve that innervates such an organ when the cumulative amount and/or duration of consumption of this selected nutrient exceeds an allowable nutrient-specific consumption amount and/or duration.

In various examples, a selected nutrient can be selected from the group consisting of: a specific sugar, a specific carbohydrate, a specific fat, a specific cholesterol, and a specific sodium compound. In various examples, a selected nutrient can be selected from the group consisting of: a category of sugars, a category of carbohydrates, a category of fats, a category of cholesterols, a category of sodium compounds, sugars, carbohydrates, fats, cholesterols, and sodium compounds.

In various examples, an intraoral specific-nutrient-identifying sensor can be configured to be implanted within the person's oral cavity or attached within the person's oral cavity. In various examples, an intraoral specific-nutrient-identifying sensor can be configured to be in fluid communication with the person's oral cavity via an artificial lumen.

In various examples, delivery of electricity by a gastrointestinal electrical stimulator can reduce the amount of food that the person consumes by creating a feeling of fullness and/or satiety. In various examples, delivery of electricity by a gastrointestinal electrical stimulator can reduce the amount of food that the person consumes by decreasing the speed at which food moves through a portion of the person's gastrointestinal tract. In various examples, delivery of electricity by a gastrointestinal electrical stimulator can reduce absorption of nutrients from food that the person consumes by increasing the speed at which food moves through a portion of the person's gastrointestinal tract.

In various examples, delivery of electricity by a gastrointestinal electrical stimulator can reduce the amount of food that the person consumes by modifying the person's sense of taste and/or smell. In various examples, delivery of electricity by a gastrointestinal electrical stimulator can reduce the amount of food that the person consumes by delivering an unpleasant electrical stimulus to the person's body in response to food consumption.

In various examples, a nutrient-specific allowable consumption amount and/or duration can depend on one or more of the following factors: the type of selected nutrient; the specificity or breadth of the selected nutrient type; the accuracy of a sensor in detecting the selected nutrient; and the speed or pace of nutrient consumption. In various examples, a nutrient-specific allowable consumption amount and/or duration can depend on one or more of the following factors: a person's age, gender, and/or weight; changes in the person's weight; a person's diagnosed health conditions; and one or more general health status indicators.

In various examples, a nutrient-specific allowable consumption amount and/or duration can depend on one or more of the following factors: the magnitude and/or certainty of the effects of past consumption of the selected nutrient on a person's health; achievement of a person's health goals; and a person's exercise patterns and/or caloric expenditure. In various examples, a nutrient-specific allowable consumption amount and/or duration can depend on one or more of the following factors: a person's physical location; the time of day; the day of the week; and occurrence of a holiday or other occasion involving special meals.

In various examples, a nutrient-specific allowable consumption amount and/or duration can depend on input from a social network, a behavioral support group, a virtual health coach, and/or a health care provider. In various examples, a nutrient-specific allowable consumption amount and/or duration can depend on one or more of the following factors: the cost of food; financial payments, constraints, and/or incentives; and health insurance copay and/or health insurance premium. In various examples, a nutrient-specific allowable consumption amount and/or duration can depend on: the amount and/or duration of a person's consumption of healthy food or nutrients; and/or a dietary plan created for a person by a health care provider.

In an example, a cumulative-nutrient-consumption regulator can be co-located with a specific-nutrient-identifying sensor or co-located with a gastrointestinal electrical stimulator.

As shown by FIGS. 1 though 6, in various examples, this invention can be embodied in a method for identifying and reducing excess consumption and/or absorption of a selected type of nutrient, comprising:

(a) monitoring to detect when there is a high probability that a person is consuming food; wherein this monitoring detects food consumption based on one or more inputs selected from the group consisting of: sounds produced by swallowing, chewing, and/or other behavior related to food consumption and/or digestion; images of food, food packaging, food containers, food labels, and/or food identification codes; images of the person's hand, fingers, wrist, arm, mouth, and/or head related to food consumption; movements of the person's hand, fingers, wrist, arm, mouth, and/or head related to food consumption; peristaltic motion, wall expansion, or other motion of a gastrointestinal organ related to food consumption; motion of food, chyme, saliva, oral fluid, and/or other material passing through the person's gastrointestinal tract; optical and/or infrared spectroscopy analysis of food, chyme, saliva, oral fluid, and/or other material within the person's gastrointestinal tract; sonic analysis of food, chyme, saliva, oral fluid, and/or other material within the person's gastrointestinal tract; chemical analysis of food, chyme, saliva, oral fluid, and/or other material within the person's gastrointestinal tract; pH level analysis of food, chyme, saliva, oral fluid, and/or other material within the person's gastrointestinal tract; pressure analysis of food, chyme, saliva, oral fluid, and/or other material within the person's gastrointestinal tract; impedance analysis of food, chyme, saliva, oral fluid, and/or other material within the person's gastrointestinal tract; electromagnetic signals from a gastrointestinal organ and/or from a nerve innervating such an organ; electromagnetic signals from neurons that receive signals from the person's organs of taste and/or smell; secretory activity of an organ that secretes a substance into the gastrointestinal tract; and the physical location of the person from a physical location identification system;

(b) if the results of this monitoring detect a high probability that the person is consuming food, then triggering or increasing analysis of the chemical composition of material in a person's oral cavity and/or gastrointestinal tract in order to selectively detect consumption of a selected nutrient or nutrient type; wherein this nutrient is selected from the group consisting of: a specific sugar, a specific carbohydrate, a specific fat, a specific cholesterol, a specific sodium compound, a category of sugars, a category of carbohydrates, a category of fats, a category of cholesterols, a category of sodium compounds, sugars, carbohydrates, fats, cholesterols, and sodium compounds;

(c) keeping track of the cumulative amount of consumption and/or duration of consumption of at least one selected nutrient as identified by the sensor; wherein the nutrient-specific allowable consumption amount and/or duration depends on one or more factors selected from the group consisting of: the type of selected nutrient; the specificity or breadth of the selected nutrient type; the accuracy of the sensor in detecting the selected nutrient; the speed or pace of nutrient consumption; the person's age, gender, and/or weight; changes in the person's weight; the person's diagnosed health conditions; one or more general health status indicators; the magnitude and/or certainty of the effects of past consumption of the selected nutrient on the person's health; achievement of the person's health goals; the person's exercise patterns and/or caloric expenditure; the person's physical location; the time of day; the day of the week; occurrence of a holiday or other occasion involving special meals; input from a social network and/or behavioral support group; input from a virtual health coach; the cost of food; financial payments, constraints, and/or incentives; health insurance copay and/or health insurance premium; the amount and/or duration of the person's consumption of healthy food or nutrients; and a dietary plan created for the person by a health care provider; and wherein this regulator is co-located with the sensor, co-located with the stimulator, and/or in a location that is separate from the sensor and stimulator; and

(d) if the cumulative amount and/or duration of consumption of this selected nutrient exceeds an allowable nutrient-specific consumption amount and/or duration, then activating a gastrointestinal electrical stimulator to deliver electricity to a gastrointestinal organ and/or nerve; wherein this stimulator is configured to be implanted within the person's body to deliver electricity to a gastrointestinal organ and/or a nerve that innervates such an organ; and wherein delivery of this electricity reduces the person's consumption and/or absorption of food by a mechanism selected from the group consisting of: reducing the amount of food that the person consumes by creating a feeling of fullness and/or satiety; reducing the amount of food that the person consumes by decreasing the speed at which food moves through a portion of the person's gastrointestinal tract; reducing absorption of nutrients from food that the person consumes by increasing the speed at which food moves through a portion of the person's gastrointestinal tract; reducing the amount of food that the person consumes by modifying the person's sense of taste and/or smell; and reducing the amount of food that the person consumes by delivering an unpleasant electrical stimulus to the person's body in response to food consumption.

As shown by FIGS. 7 and 8, in various examples, this invention can be embodied in a device for reducing excess consumption of a selected nutrient, comprising:

(a) a generic-food-consumption monitor; wherein this monitor detects when there is a high probability that a person is consuming food; wherein this monitor detects food consumption based on one or more inputs selected from the group consisting of: sounds produced by swallowing, chewing, and/or other behavior related to food consumption and/or digestion; images of food, food packaging, food containers, food labels, and/or food identification codes; images of the person's hand, fingers, wrist, arm, mouth, and/or head related to food consumption; movements of the person's hand, fingers, wrist, arm, mouth, and/or head related to food consumption; peristaltic motion, wall expansion, or other motion of a gastrointestinal organ related to food consumption; motion of food, chyme, saliva, oral fluid, and/or other material passing through the person's gastrointestinal tract; optical and/or infrared spectroscopy analysis of food, chyme, saliva, oral fluid, and/or other material within the person's gastrointestinal tract; sonic analysis of food, chyme, saliva, oral fluid, and/or other material within the person's gastrointestinal tract; chemical analysis of food, chyme, saliva, oral fluid, and/or other material within the person's gastrointestinal tract; pH level analysis of food, chyme, saliva, oral fluid, and/or other material within the person's gastrointestinal tract; pressure analysis of food, chyme, saliva, oral fluid, and/or other material within the person's gastrointestinal tract; impedance analysis of food, chyme, saliva, oral fluid, and/or other material within the person's gastrointestinal tract; electromagnetic signals from a gastrointestinal organ and/or from a nerve innervating such an organ; electromagnetic signals from neurons that receive signals from the person's organs of taste and/or smell; secretory activity of an organ that secretes a substance into the gastrointestinal tract; and the physical location of the person using a physical location identification system;

(b) a specific-nutrient-identifying sensor; wherein operation of this sensor is triggered or increased when there is a high probability of food consumption based on results from the generic-food-consumption monitor; wherein this sensor automatically analyzes the chemical composition of material in a person's oral cavity and/or gastrointestinal tract in order to selectively identify the person's consumption of at least one selected nutrient; and wherein this nutrient is selected from the group consisting of: a specific sugar, a specific carbohydrate, a specific fat, a specific cholesterol, a specific sodium compound, a category of sugars, a category of carbohydrates, a category of fats, a category of cholesterols, a category of sodium compounds, sugars, carbohydrates, fats, cholesterols, and sodium compounds;

(c) a gastrointestinal electrical stimulator; wherein this stimulator is configured to be implanted within the person's body to deliver electricity to a gastrointestinal organ and/or a nerve that innervates such an organ; and wherein delivery of this electricity reduces the person's consumption and/or absorption of food by a mechanism selected from the group consisting of: reducing the amount of food that the person consumes by creating a feeling of fullness and/or satiety; reducing the amount of food that the person consumes by decreasing the speed at which food moves through a portion of the person's gastrointestinal tract; reducing absorption of nutrients from food that the person consumes by increasing the speed at which food moves through a portion of the person's gastrointestinal tract; reducing the amount of food that the person consumes by modifying the person's sense of taste and/or smell; and reducing the amount of food that the person consumes by delivering an unpleasant electrical stimulus to the person's body in response to food consumption; and

(d) a cumulative-nutrient-consumption regulator; wherein this regulator keeps track of the cumulative amount of consumption and/or duration of consumption of at least one selected nutrient as identified by the sensor and triggers the stimulator to deliver electricity to a gastrointestinal organ and/or a nerve that innervates such an organ when the cumulative amount and/or duration of consumption of this selected nutrient exceeds an allowable nutrient-specific consumption amount and/or duration; wherein the nutrient-specific allowable consumption amount and/or duration depends on one or more factors selected from the group consisting of: the type of selected nutrient; the specificity or breadth of the selected nutrient type; the accuracy of the sensor in detecting the selected nutrient; the speed or pace of nutrient consumption; the person's age, gender, and/or weight; changes in the person's weight; the person's diagnosed health conditions; one or more general health status indicators; the magnitude and/or certainty of the effects of past consumption of the selected nutrient on the person's health; achievement of the person's health goals; the person's exercise patterns and/or caloric expenditure; the person's physical location; the time of day; the day of the week; occurrence of a holiday or other occasion involving special meals; input from a social network and/or behavioral support group; input from a virtual health coach; the cost of food; financial payments, constraints, and/or incentives; health insurance copay and/or health insurance premium; the amount and/or duration of the person's consumption of healthy food or nutrients; and a dietary plan created for the person by a health care provider; and wherein this regulator is co-located with the sensor, co-located with the stimulator, and/or in a location that is separate from the sensor and stimulator.

I claim:

1. A device for reducing excess consumption and/or absorption of a selected nutrient, comprising:
an intraoral specific-nutrient-identifying sensor; wherein this sensor automatically analyzes the chemical composition of food, saliva, and/or oral fluid in a person's oral cavity in order to selectively identify the person's consumption of at least one selected nutrient;
a gastrointestinal electrical stimulator; wherein this stimulator is configured to be implanted within the person's body to deliver electricity to a gastrointestinal organ and/or a nerve that innervates such an organ; and
a cumulative-nutrient-consumption regulator; wherein this regulator keeps track of the cumulative amount of consumption and/or duration of consumption of a selected nutrient as identified by the specific-nutrient-identifying sensor and triggers the gastrointestinal electrical stimulator to deliver electricity to a gastrointestinal organ and/or to a nerve that innervates such an organ when the cumulative amount and/or duration of consumption of this selected nutrient exceeds an allowable nutrient-specific consumption amount and/or duration.

2. The device in claim 1 wherein the selected nutrient is selected from the group consisting of: a specific sugar, a specific carbohydrate, a specific fat, a specific cholesterol, and a specific sodium compound.

3. The device in claim 1 wherein the selected nutrient is selected from the group consisting of: a category of sugars, a category of carbohydrates, a category of fats, a category of cholesterols, a category of sodium compounds, sugars, carbohydrates, fats, cholesterols, and sodium compounds.

4. The device in claim 1 wherein the intraoral specific-nutrient-identifying sensor is configured to be implanted within the person's oral cavity or attached within the person's oral cavity.

5. The device in claim 1 wherein the intraoral specific-nutrient-identifying sensor is configured to be in fluid communication with the person's oral cavity via an artificial lumen.

6. The device in claim 1 wherein delivery of electricity by the gastrointestinal electrical stimulator reduces the amount of food that the person consumes by creating a feeling of fullness and/or satiety.

7. The device in claim 1 wherein delivery of electricity by the gastrointestinal electrical stimulator reduces the amount of food that the person consumes by decreasing the speed at which food moves through a portion of the person's gastrointestinal tract.

8. The device in claim 1 wherein delivery of electricity by the gastrointestinal electrical stimulator reduces absorption of nutrients from food that the person consumes by increasing the speed at which food moves through a portion of the person's gastrointestinal tract.

9. The device in claim 1 wherein delivery of electricity by the gastrointestinal electrical stimulator reduces the amount of food that the person consumes by modifying the person's sense of taste and/or smell.

10. The device in claim 1 wherein delivery of electricity by the gastrointestinal electrical stimulator reduces the amount of food that the person consumes by delivering an unpleasant electrical stimulus to the person's body in response to food consumption.

11. The device in claim 1 wherein the nutrient-specific allowable consumption amount and/or duration depends on one or more of the following factors: the type of selected nutrient; the specificity or breadth of the selected nutrient type; the accuracy of the sensor in detecting the selected nutrient; and the speed or pace of nutrient consumption.

12. The device in claim 1 wherein the nutrient-specific allowable consumption amount and/or duration depends on one or more of the following factors: the person's age, gender, and/or weight; changes in the person's weight; the person's diagnosed health conditions; and one or more general health status indicators.

13. The device in claim 1 wherein the nutrient-specific allowable consumption amount and/or duration depends on one or more of the following factors: the magnitude and/or certainty of the effects of past consumption of the selected nutrient on the person's health; achievement of the person's health goals; and the person's exercise patterns and/or caloric expenditure.

14. The device in claim 1 wherein the nutrient-specific allowable consumption amount and/or duration depends on one or more of the following factors: the person's physical location; the time of day; the day of the week; and occurrence of a holiday or other occasion involving special meals.

15. The device in claim 1 wherein the nutrient-specific allowable consumption amount and/or duration depends on input from a social network, a behavioral support group, a virtual health coach, and/or a health care provider.

16. The device in claim 1 wherein the nutrient-specific allowable consumption amount and/or duration depends on one or more of the following factors: the cost of food; financial payments, constraints, and/or incentives; and health insurance copay and/or health insurance premium.

17. The device in claim 1 wherein the nutrient-specific allowable consumption amount and/or duration depends on: the amount and/or duration of the person's consumption of healthy food or nutrients; and/or a dietary plan created for the person by a health care provider.

18. The device in claim 1 wherein the cumulative-nutrient-consumption regulator is co-located with the sensor or co-located with the stimulator.

19. A device for reducing excess consumption of a selected nutrient, comprising:
a generic-food-consumption monitor; wherein this monitor detects when there is a high probability that a person is consuming food; wherein this monitor detects food consumption based on one or more inputs selected from the group consisting of: sounds produced by swallowing, chewing, and/or other behavior related to food consumption and/or digestion; images of food, food packaging, food containers, food labels, and/or food identification codes; images of the person's hand, fingers, wrist, arm, mouth, and/or head related to food consumption; movements of the person's hand, fingers, wrist, arm, mouth, and/or head related to food consumption; peristaltic motion, wall expansion, or other motion of a gastrointestinal organ related to food consumption; motion of food, chyme, saliva, oral fluid, and/or other material passing through the person's gastrointestinal tract; optical and/or infrared spectroscopy analysis of food, chyme, saliva, oral fluid, and/or other material within the person's gastrointestinal tract; sonic analysis of food, chyme, saliva, oral fluid, and/or other material within the person's gastrointestinal tract; chemical analysis of food, chyme, saliva, oral fluid, and/or other material within the person's gastrointestinal tract; pH level analysis of food, chyme, saliva, oral fluid, and/or other material within the person's gastrointestinal tract; pressure analysis of food, chyme, saliva, oral fluid, and/or other material within the person's gastrointestinal tract; impedance analysis of food, chyme, saliva, oral fluid, and/or other material within the person's gastrointestinal tract; electromagnetic signals from a gastrointestinal organ and/or from a nerve innervating such an organ; electromagnetic signals from neurons that receive signals from the person's organs of taste and/or smell; secretory activity of an organ that secretes a substance into the gastrointestinal tract; and the physical location of the person using a physical location identification system;
a specific-nutrient-identifying sensor; wherein operation of this sensor is triggered or increased when there is a high probability of food consumption based on results from the generic-food-consumption monitor; wherein this sensor automatically analyzes the chemical composition of material in a person's oral cavity and/or gastrointestinal tract in order to selectively identify the person's consumption of at least one selected nutrient; and wherein this nutrient is selected from the group consisting of: a specific sugar, a specific carbohydrate, a specific fat, a specific cholesterol, a specific sodium compound, a category of sugars, a category of carbohydrates, a category of fats, a category of cholesterols, a category of sodium compounds, sugars, carbohydrates, fats, cholesterols, and sodium compounds;
a gastrointestinal electrical stimulator; wherein this stimulator is configured to be implanted within the person's body to deliver electricity to a gastrointestinal organ and/or a nerve that innervates such an organ; and wherein delivery of this electricity reduces the person's consumption and/or absorption of food by a mechanism selected from the group consisting of: reducing the amount of food that the person consumes by creating a feeling of fullness and/or satiety; reducing the amount of food that the person consumes by decreasing the speed at which food moves through a portion of the person's gastrointestinal tract; reducing absorption of nutrients from food that the person consumes by increasing the speed at which food moves through a portion of the person's gastrointestinal tract; reducing the amount of food that the person consumes by modifying the person's sense of taste and/or smell; and reducing the amount of food that the person consumes by delivering an unpleasant electrical stimulus to the person's body in response to food consumption; and
a cumulative-nutrient-consumption regulator; wherein this regulator keeps track of the cumulative amount of consumption and/or duration of consumption of at least one selected nutrient as identified by the sensor and triggers the stimulator to deliver electricity to a gastrointestinal organ and/or a nerve that innervates such an organ when the cumulative amount and/or duration of consumption of this selected nutrient exceeds an allowable nutrient-specific consumption amount and/or duration; wherein the nutrient-specific allowable consumption amount and/or duration depends on one or more factors selected from the group consisting of: the type of selected nutrient; the specificity or breadth of the selected nutrient type; the accuracy of the sensor in detecting the selected nutrient; the speed or pace of nutrient consumption; the person's age, gender, and/or weight; changes in the person's weight; the person's diagnosed health conditions; one or more general health status indicators; the magnitude and/or certainty of the effects of past consumption of the selected nutrient on the person's health; achievement of the person's health goals; the person's exercise patterns and/or caloric expenditure; the person's physical location; the time of day; the day of the week; occurrence of a holiday or other occasion involving special meals; input from a social network and/or behavioral support group; input from a virtual health coach; the cost of food; financial payments, constraints, and/or incentives; health insurance copay and/or health insurance premium; the amount and/or duration of the person's consumption of healthy food or nutrients; and a dietary plan created for the person by a health care provider; and wherein this regulator is co-located with the sensor, co-located with the stimulator, and/or in a location that is separate from the sensor and stimulator.

20. A method for identifying and reducing excess consumption and/or absorption of a selected type of nutrient, comprising:
monitoring to detect when there is a high probability that a person is consuming food; wherein this monitoring detects food consumption based on one or more inputs selected from the group consisting of: sounds produced by swallowing, chewing, and/or other behavior related to food consumption and/or digestion; images of food, food packaging, food containers, food labels, and/or food identification codes; images of the person's hand, fingers, wrist, arm, mouth, and/or head related to food consumption; movements of the person's hand, fingers, wrist, arm, mouth, and/or head related to food consumption; peristaltic motion, wall expansion, or other motion of a gastrointestinal organ related to food consumption; motion of food, chyme, saliva, oral fluid, and/or other material passing through the person's gastrointestinal tract; optical and/or infrared spectroscopy analysis of food, chyme, saliva, oral fluid, and/or other material within the person's gastrointestinal tract; sonic analysis of food, chyme, saliva, oral fluid, and/or other material within the person's gastrointestinal tract; chemical analysis of food, chyme, saliva, oral fluid, and/or other material within the person's gastrointestinal tract; pH level analysis of food, chyme, saliva, oral fluid, and/or other material within the person's gastrointestinal tract; pressure analysis of food, chyme, saliva, oral fluid, and/or other material within the person's gastrointestinal tract; impedance analysis of food, chyme, saliva, oral fluid, and/or other material within the person's gastrointestinal tract; electromagnetic signals from a gastrointestinal organ and/or from a nerve innervating such an organ; electromagnetic signals from neurons that receive signals from the person's organs of taste and/or smell; secretory activity of an organ that secretes a substance into the gastrointestinal tract; and the physical location of the person from a physical location identification system;
if the results of this monitoring detect a high probability that the person is consuming food, then triggering or increasing analysis of the chemical composition of material in a person's oral cavity and/or gastrointestinal tract in order to selectively detect consumption of a selected nutrient or nutrient type; wherein this nutrient is selected from the group consisting of: a specific sugar, a specific carbohydrate, a specific fat, a specific cholesterol, a specific sodium compound, a category of sugars, a category of carbohydrates, a category of fats, a category of cholesterols, a category of sodium compounds, sugars, carbohydrates, fats, cholesterols, and sodium compounds;
keeping track of the cumulative amount of consumption and/or duration of consumption of at least one selected nutrient as identified by the sensor; wherein the nutrient-specific allowable consumption amount and/or duration depends on one or more factors selected from the group consisting of: the type of selected nutrient; the specificity or breadth of the selected nutrient type; the accuracy of the sensor in detecting the selected nutrient; the speed or pace of nutrient consumption; the person's age, gender, and/or weight; changes in the person's weight; the person's diagnosed health conditions; one or more general health status indicators; the magnitude and/or certainty of the effects of past consumption of the selected nutrient on the person's health; achievement of the person's health goals; the person's exercise patterns and/or caloric expenditure; the person's physical location; the time of day; the day of the week; occurrence of a holiday or other occasion involving special meals; input from a social network and/or behavioral support group; input from a virtual health coach; the cost of food; financial payments, constraints, and/or incentives; health insurance copay and/or health insurance premium; the amount and/or duration of the person's consumption of healthy food or nutrients; and a dietary plan created for the person by a health care provider; and wherein this regulator is co-located with the sensor, co-located with the stimulator, and/or in a location that is separate from the sensor and stimulator; and
if the cumulative amount and/or duration of consumption of this selected nutrient exceeds an allowable nutrient-specific consumption amount and/or duration, then activating a gastrointestinal electrical stimulator to deliver electricity to a gastrointestinal organ and/or nerve; wherein this stimulator is configured to be implanted within the person's body to deliver electricity to a gastrointestinal organ and/or a nerve that innervates such an organ; and wherein delivery of this electricity reduces the person's consumption and/or absorption of food by a mechanism selected from the group consisting of: reducing the amount of food that the person consumes by creating a feeling of fullness and/or satiety; reducing the amount of food that the person consumes by decreasing the speed at which food moves through a portion of the person's gastrointestinal tract; reducing absorption of nutrients from food that the person consumes by increasing the speed at which food moves through a portion of the person's gastrointestinal tract; reducing the amount of food that the person consumes by modifying the person's sense of taste and/or smell; and reducing the amount of food that the person consumes by delivering an unpleasant electrical stimulus to the person's body in response to food consumption.
